SEQUENCE LISTING (308 sequences)

SEQ ID NO: 1 - protein, 153 aa, synthetic construct
  REGION 1..153: note = Human IL-2 Sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, synthetic construct [sequence omitted]

SEQ ID NO: 3 - protein, 272 aa, synthetic construct
  REGION 1..272: note = Human IL-2R (CD25) sequence
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 4 - protein, 219 aa, synthetic construct
  REGION 1..219: note = Human IL-2R (CD25), extracellular domain
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 5 - protein, 551 aa, synthetic construct
  REGION 1..551: note = Human IL-2R (CD122) sequence
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 214 aa, synthetic construct
  REGION 1..214: note = Human IL-2R (CD122), extracellular domain
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 7 - protein, 369 aa, synthetic construct
  REGION 1..369: note = Human common gamma chain (CD132) sequence
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 8 - protein, 240 aa, synthetic construct
  REGION 1..240: note = Human common gamma chain (CD132), extracellular domain
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 9 - protein, 231 aa, synthetic construct
  REGION 1..231: note = IL-2-Fc monomer 1
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 231 aa, synthetic construct
  REGION 1..231: note = IL-2-Fc monomer 2
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 11 - protein, 231 aa, synthetic construct
  REGION 1..231: note = IL-2-Fc monomer 1
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 231 aa, synthetic construct; identical to SEQ ID NO: 10
  REGION 1..231: note = IL-2-Fc monomer 2

SEQ ID NO: 13 - protein, 231 aa, synthetic construct; identical to SEQ ID NO: 11
  REGION 1..231: note = IL-2-Fc monomer 1

SEQ ID NO: 14 - protein, 231 aa, synthetic construct
  REGION 1..231: note = IL-2-Fc monomer 2
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 15 - protein, 231 aa, synthetic construct
  REGION 1..231: note = IL-2-Fc monomer 1
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 16 - protein, 231 aa, synthetic construct
  REGION 1..231: note = IL-2-Fc monomer 2
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 17 - protein, 231 aa, synthetic construct
  REGION 1..231: note = IL-2-Fc monomer 1
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 231 aa, synthetic construct
  REGION 1..231: note = IL-2-Fc monomer 2
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 19 - protein, 231 aa, synthetic construct
  REGION 1..231: note = IL-2-Fc monomer 1
EPKSSDKTHTCPPCPAPPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 231 aa, synthetic construct
  REGION 1..231: note = IL-2-Fc monomer 2
EPKSSDKTHTCPPCPAPPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 21 - protein, 231 aa, synthetic construct
  REGION 1..231: note = IL-2-Fc monomer 1
EPKSSDKTHTCPPCPAPPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 231 aa, synthetic construct
  REGION 1..231: note = IL-2-Fc monomer
EPKSSDKTHTCPPCPAPPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 23 - protein, 229 aa, synthetic construct
  REGION 1..229: note = IL-2-Fc monomer 1
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 229 aa, synthetic construct
  REGION 1..229: note = IL-2-Fc monomer 2
ESKYGPPCPPCPAPEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 25 - protein, 228 aa, synthetic construct
  REGION 1..228: note = IL-2-Fc monomer 1
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 228 aa, synthetic construct
  REGION 1..228: note = IL-2-Fc monomer 2
ERKCCVECPPCPAPPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 27 - protein, 228 aa, synthetic construct
  REGION 1..228: note = IL-2-Fc monomer 1
ERKCCVECPPCPAPPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 228 aa, synthetic construct
  REGION 1..228: note = IL-2-Fc monomer 2
ERKCCVECPPCPAPPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 29 - protein, 231 aa, synthetic construct
  REGION 1..231: note = IL-2-Fc monomer 1
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 30 - protein, 231 aa, synthetic construct
  REGION 1..231: note = IL-2-Fc monomer 2
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 31 - protein, 231 aa, synthetic construct
  REGION 1..231: note = IL-2-Fc monomer 1
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 231 aa, synthetic construct; identical to SEQ ID NO: 31
  REGION 1..231: note = IL-2-Fc monomer 2

SEQ ID NO: 33 - protein, 231 aa, synthetic construct; identical to SEQ ID NO: 10
  REGION 1..231: note = IL-2-Fc monomer 1

SEQ ID NO: 34 - protein, 231 aa, synthetic construct
  REGION 1..231: note = IL-2-Fc monomer 2
ERKSSDKTHTCPRCPAPPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 5 aa, synthetic construct
  REGION 1..5: note = linker
GGGGS

SEQ ID NO: 36 - protein, 10 aa, synthetic construct
  REGION 1..10: note = linker
GGGGSGGGGS

SEQ ID NO: 37 - protein, 15 aa, synthetic construct
  REGION 1..15: note = linker
GGGGSGGGGSGGGGS

SEQ ID NO: 38 - protein, 20 aa, synthetic construct
  REGION 1..20: note = linker
GGGGSGGGGSGGGGSGGGGS

SEQ ID NO: 39 - protein, 5 aa, synthetic construct
  REGION 1..5: note = linker
GGGES

SEQ ID NO: 40 - protein, 135 aa, synthetic construct
  REGION 1..135: note = XENP014135 hIL2_0.1
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 41 - protein, 135 aa, synthetic construct
  REGION 1..135: note = XENP014136 hIL2_0.9
APTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 42 - protein, 133 aa, synthetic construct
  REGION 1..133: note = XENP014137 hIL2_0.10
APTSSSTKKTQLQLEHLLLDLQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 43 - protein, 133 aa, synthetic construct
  REGION 1..133: note = XENP014138 hIL2_0.11
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 44 - protein, 133 aa, synthetic construct
  REGION 1..133: note = XENP014139 hIL2_0.12
APTSSSTKKTQLQLEHLLLDLQMILNGINNYKNPKLTFMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 45 - protein, 133 aa, synthetic construct
  REGION 1..133: note = XENP014140 hIL2_0.13
APTSSSTKKTQLQLEHLLLDLQMILNGINNYKNPKLTGMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 46 - protein, 133 aa, synthetic construct
  REGION 1..133: note = XENP014141 hIL2_0.14
APTSSSTKKTQLQLEHLLLDLQMILNGINNYKNPKLTHMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 47 - protein, 135 aa, synthetic construct
  REGION 1..135: note = XENP014142 hIL2_0.15
APTSSSTKKTQLQLEHLLLDLQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 48 - protein, 133 aa, synthetic construct
  REGION 1..133: note = XENP014143 hIL2_0.16
APTSSSTKKTQLQLEHLLLDLQMILNGINNYKNPKLTKMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 49 - protein, 135 aa, synthetic construct
  REGION 1..135: note = XENP014144 hIL2_0.17
APTSSSTKKTQLQLEHLLLDLQMILNGINNYKNPKLTLMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 50 - protein, 133 aa, synthetic construct
  REGION 1..133: note = XENP014145 hIL2_0.18
APTSSSTKKTQLQLEHLLLDLQMILNGINNYKNPKLTMMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 51 - protein, 133 aa, synthetic construct
  REGION 1..133: note = XENP014146 hIL2_0.19
APTSSSTKKTQLQLEHLLLDLQMILNGINNYKNPKLTNMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 52 - protein, 133 aa, synthetic construct
  REGION 1..133: note = XENP014147 hIL2_0.20
APTSSSTKKTQLQLEHLLLDLQMILNGINNYKNPKLTPMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 53 - protein, 133 aa, synthetic construct
  REGION 1..133: note = XENP014148 hIL2_0.21
APTSSSTKKTQLQLEHLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 54 - protein, 133 aa, synthetic construct
  REGION 1..133: note = XENP014149 hIL2_0.22
APTSSSTKKTQLQLEHLLLDLQMILNGINNYKNPKLTSMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 55 - protein, 133 aa, synthetic construct
  REGION 1..133: note = XENP014150 hIL2_0.23
APTSSSTKKTQLQLEHLLLDLQMILNGINNYKNPKLTTMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 56 - protein, 133 aa, synthetic construct
  REGION 1..133: note = XENP014151 hIL2_0.24
APTSSSTKKTQLQLEHLLLDLQMILNGINNYKNPKLTVMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 57 - protein, 133 aa, synthetic construct
  REGION 1..133: note = XENP014152 hIL2_0.25
APTSSSTKKTQLQLEHLLLDLQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 58 - protein, 133 aa, synthetic construct
  REGION 1..133: note = XENP014153 hIL2_0.26
APTSSSTKKTQLQLEHLLLDLQMILNGINNYKNPKLTYMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 59 - protein, 133 aa, synthetic construct
  REGION 1..133: note = XENP014154 hIL2_0.27
APTSSSTKKTQLQLEHLLLDLQMILNGINNYKNPKLTRMLAFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 60 - protein, 133 aa, synthetic construct
  REGION 1..133: note = XENP014155 hIL2_0.28
APTSSSTKKTQLQLEHLLLDLQMILNGINNYKNPKLTRMLDFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 61 - protein, 133 aa, synthetic construct
  REGION 1..133: note = XENP014156 hIL2_0.29
APTSSSTKKTQLQLEHLLLDLQMILNGINNYKNPKLTRMLEFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 62 - protein, 133 aa, synthetic construct
  REGION 1..133: note = XENP014157 hIL2_0.30
APTSSSTKKTQLQLEHLLLDLQMILNGINNYKNPKLTRMLFFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 63 - protein, 133 aa, synthetic construct
  REGION 1..133: note = XENP014158 hIL2_0.31
APTSSSTKKTQLQLEHLLLDLQMILNGINNYKNPKLTRMLGFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 64 - protein, 133 aa, synthetic construct
  REGION 1..133: note = XENP014159 hIL2_0.32
APTSSSTKKTQLQLEHLLLDLQMILNGINNYKNPKLTRMLHFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 65 - protein, 133 aa, synthetic construct
  REGION 1..133: note = XENP014160 hIL2_0.33
APTSSSTKKTQLQLEHLLLDLQMILNGINNYKNPKLTRMLIFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 66 - protein, 133 aa, synthetic construct
  REGION 1..133: note = XENP014161 hIL2_0.34
APTSSSTKKTQLQLEHLLLDLQMILNGINNYKNPKLTRMLKFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 67 - protein, 133 aa, synthetic construct
  REGION 1..133: note = XENP014162 hIL2_0.35
APTSSSTKKTQLQLEHLLLDLQMILNGINNYKNPKLTRMLLFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 68 - protein, 133 aa, synthetic construct
  REGION 1..133: note = XENP014163 hIL2_0.36
APTSSSTKKTQLQLEHLLLDLQMILNGINNYKNPKLTRMLMFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 69 - protein, 133 aa, synthetic construct
  REGION 1..133: note = XENP014164 hIL2_0.37
APTSSSTKKTQLQLEHLLLDLQMILNGINNYKNPKLTRMLNFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 70 - protein, 133 aa, synthetic construct
  REGION 1..133: note = XENP014165 hIL2_0.38
APTSSSTKKTQLQLEHLLLDLQMILNGINNYKNPKLTRMLPFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 71 - protein, 133 aa, synthetic construct
  REGION 1..133: note = XENP014166 hIL2_0.39
APTSSSTKKTQLQLEHLLLDLQMILNGINNYKNPKLTRMLQFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 72 - protein, 133 aa, synthetic construct
  REGION 1..133: note = XENP014167 hIL2_0.40
APTSSSTKKTQLQLEHLLLDLQMILNGINNYKNPKLTRMLRFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 73 - protein, 133 aa, synthetic construct
  REGION 1..133: note = XENP014168 hIL2_0.41
APTSSSTKKTQLQLEHLLLDLQMILNGINNYKNPKLTRMLSFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 74 - protein, 133 aa, synthetic construct
  REGION 1..133: note = XENP014169 hIL2_0.42
APTSSSTKKTQLQLEHLLLDLQMILNGINNYKNPKLTRMLVFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 75 - protein, 133 aa, synthetic construct
  REGION 1..133: note = XENP014170 hIL2_0.43
APTSSSTKKTQLQLEHLLLDLQMILNGINNYKNPKLTRMLWFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 76 - protein, 133 aa, synthetic construct
  REGION 1..133: note = XENP014171 hIL2_0.44
APTSSSTKKTQLQLEHLLLDLQMILNGINNYKNPKLTRMLYFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 77 - protein, 133 aa, synthetic construct
  REGION 1..133: note = XENP014172 hIL2_0.45
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 78 - protein, 133 aa, synthetic construct
  REGION 1..133: note = XENP014173 hIL2_0.46
APTSSSTKKTQLQLEHLLLDLQMILNGINNYKNPKLTRMLTDKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 79 - protein, 133 aa, synthetic construct
  REGION 1..133: note = XENP014174 hIL2_0.47
APTSSSTKKTQLQLEHLLLDLQMILNGINNYKNPKLTRMLTEKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 80 - protein, 133 aa, synthetic construct
  REGION 1..133: note = XENP014175 hIL2_0.48
APTSSSTKKTQLQLEHLLLDLQMILNGINNYKNPKLTRMLTGKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 81 - protein, 133 aa, synthetic construct
  REGION 1..133: note = XENP014176 hIL2_0.49
APTSSSTKKTQLQLEHLLLDLQMILNGINNYKNPKLTRMLTHKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 82 - protein, 133 aa, synthetic construct
  REGION 1..133: note = XENP014177 hIL2_0.50
APTSSSTKKTQLQLEHLLLDLQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 83 - protein, 133 aa, synthetic construct
  REGION 1..133: note = XENP014178 hIL2_0.51
APTSSSTKKTQLQLEHLLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 84 - protein, 133 aa, synthetic construct
  REGION 1..133: note = XENP014179 hIL2_0.52
APTSSSTKKTQLQLEHLLLDLQMILNGINNYKNPKLTRMLTLKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 85 - protein, 133 aa, synthetic construct
  REGION 1..133: note = XENP014180 hIL2_0.53
APTSSSTKKTQLQLEHLLLDLQMILNGINNYKNPKLTRMLTMKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 86 - protein, 129 aa, synthetic construct
  REGION 1..129: note = XENP014181 hIL2_0.54
APTSSSTKKTQLQLEHLLLDLQMILNGINNYKNPKLTRMLTNKFYMPKKATELKHLQCLEEELKPLEEVLNLAQSKNFHLRPRDLISNINVIVLELKGSETTFMCEYADETATIVEFLNRWITFSQSII

SEQ ID NO: 87 - protein, 133 aa, synthetic construct
  REGION 1..133: note = XENP014182 hIL2_0.55
APTSSSTKKTQLQLEHLLLDLQMILNGINNYKNPKLTRMLTPKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 88 - protein, 133 aa, synthetic construct
  REGION 1..133: note = XENP014183 hIL2_0.56
APTSSSTKKTQLQLEHLLLDLQMILNGINNYKNPKLTRMLTQKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 89 - protein, 133 aa, synthetic construct
  REGION 1..133: note = XENP014184 hIL2_0.57
APTSSSTKKTQLQLEHLLLDLQMILNGINNYKNPKLTRMLTRKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 90 - protein, 133 aa, synthetic construct
  REGION 1..133: note = XENP014185 hIL2_0.58
APTSSSTKKTQLQLEHLLLDLQMILNGINNYKNPKLTRMLTSKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 91 - protein, 133 aa, synthetic construct
  REGION 1..133: note = XENP014186 hIL2_0.59
APTSSSTKKTQLQLEHLLLDLQMILNGINNYKNPKLTRMLTTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 92 - protein, 133 aa, synthetic construct
  REGION 1..133: note = XENP014187 hIL2_0.60
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 93 - protein, 133 aa, synthetic construct
  REGION 1..133: note = XENP014188 hIL2_0.61
APTSSSTKKTQLQLEHLLLDLQMILNGINNYKNPKLTRMLTWKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 94 - protein, 133 aa, synthetic construct
  REGION 1..133: note = XENP014189 hIL2_0.62
APTSSSTKKTQLQLEHLLLDLQMILNGINNYKNPKLTRMLTYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 95 - protein, 133 aa, synthetic construct
  REGION 1..133: note = XENP014190 hIL2_0.63
APTSSSTKKTQLQLEHLLLDLQMILNGINNYKNPKLTQMLKFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 96 - protein, 133 aa, synthetic construct
  REGION 1..133: note = XENP014191 hIL2_0.64
APTSSSTKKTQLQLEHLLLDLQMILNGINNYKNPKLTQMLQFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 97 - protein, 133 aa, synthetic construct
  REGION 1..133: note = XENP014192 hIL2_0.65
APTSSSTKKTQLQLEHLLLDLQMILNGINNYKNPKLTEMLKFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 98 - protein, 133 aa, synthetic construct
  REGION 1..133: note = XENP014193 hIL2_0.66
APTSSSTKKTQLQLEHLLLDLQMILNGINNYKNPKLTQMLRFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 99 - protein, 133 aa, synthetic construct
  REGION 1..133: note = XENP014194 hIL2_0.67
APTSSSTKKTQLQLEHLLLDLQMILNGINNYKNPKLTNMLQFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 100 - protein, 133 aa, synthetic construct
  REGION 1..133: note = XENP014195 hIL2_0.68
APTSSSTKKTQLQLEHLLLDLQMILNGINNYKNPKLTQMLVFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 101 - protein, 133 aa, synthetic construct
  REGION 1..133: note = XENP014196 hIL2_0.69
APTSSSTKKTQLQLEHLLLDLQMILNGINNYKNPKLTNMLVFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 102 - protein, 133 aa, synthetic construct
  REGION 1..133: note = XENP014201 hIL2_0.70
APTSSSTKKTQLQLEHLLLDLQMILNGINNYKNPKLTQMLMFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 103 - protein, 133 aa, synthetic construct
  REGION 1..133: note = XENP014202 hIL2_0.71
APTSSSTKKTQLQLEHLLLDLQMILNGINNYKNPKLTQMLSFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 104 - protein, 133 aa, synthetic construct
  REGION 1..133: note = XENP014203 hIL2_0.72
APTSSSTKKTQLQLEHLLLDLQMILNGINNYKNPKLTQMLLFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 105 - protein, 133 aa, synthetic construct
  REGION 1..133: note = XENP014204 hIL2_0.73
APTSSSTKKTQLQLEHLLLDLQMILNGINNYKNPKLTNMLMFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 106 - protein, 133 aa, synthetic construct
  REGION 1..133: note = XENP014205 hIL2_0.74
APTSSSTKKTQLQLEHLLLDLQMILNGINNYKNPKLTRMLIYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 107 - protein, 133 aa, synthetic construct
  REGION 1..133: note = XENP014206 hIL2_0.75
APTSSSTKKTQLQLEHLLLDLQMILNGINNYKNPKLTRMLEYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 108 - protein, 133 aa, synthetic construct
  REGION 1..133: note = XENP014207 hIL2_0.76
APTSSSTKKTQLQLEHLLLDLQMILNGINNYKNPKLTRMLDYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 109 - protein, 133 aa, synthetic construct
  REGION 1..133: note = XENP014208 hIL2_0.77
APTSSSTKKTQLQLEHLLLDLQMILNGINNYKNPKLTRMLMYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 110 - protein, 133 aa, synthetic construct
  REGION 1..133: note = XENP014209 hIL2_0.78
APTSSSTKKTQLQLEHLLLDLQMILNGINNYKNPKLTRMLQYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 111 - protein, 133 aa, synthetic construct
  REGION 1..133: note = XENP014210 hIL2_0.79
APTSSSTKKTQLQLEHLLLDLQMILNGINNYKNPKLTRMLEHKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 112 - protein, 133 aa, synthetic construct
  REGION 1..133: note = XENP014211 hIL2_0.80
APTSSSTKKTQLQLEHLLLDLQMILNGINNYKNPKLTRMLELKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 113 - protein, 133 aa, synthetic construct
  REGION 1..133: note = XENP014212 hIL2_0.81
APTSSSTKKTQLQLEHLLLDLQMILNGINNYKNPKLTRMLEPKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 114 - protein, 133 aa, synthetic construct
  REGION 1..133: note = XENP014213 hIL2_0.82
APTSSSTKKTQLQLEHLLLDLQMILNGINNYKNPKLTQMLTYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 115 - protein, 133 aa, synthetic construct
  REGION 1..133: note = XENP014214 hIL2_0.83
APTSSSTKKTQLQLEHLLLDLQMILNGINNYKNPKLTNMLRFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 116 - protein, 133 aa, synthetic construct
  REGION 1..133: note = XENP014215 hIL2_0.84
APTSSSTKKTQLQLEHLLLDLQMILNGINNYKNPKLTNMLKFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 117 - protein, 133 aa, synthetic construct
  REGION 1..133: note = XENP014216 hIL2_0.85
APTSSSTKKTQLQLEHLLLDLQMILNGINNYKNPKLTVMLRFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 118 - protein, 133 aa, synthetic construct
  REGION 1..133: note = XENP014217 hIL2_0.86
APTSSSTKKTQLQLEHLLLDLQMILNGINNYKNPKLTPMLRFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 119 - protein, 133 aa, synthetic construct
  REGION 1..133: note = XENP014218 hIL2_0.87
APTSSSTKKTQLQLEHLLLDLQMILNGINNYKNPKLTRMLEKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 120 - protein, 133 aa, synthetic construct
  REGION 1..133: note = XENP014219 hIL2_0.88
APTSSSTKKTQLQLEHLLLDLQMILNGINNYKNPKLTRMLDKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 121 - protein, 133 aa, synthetic construct
  REGION 1..133: note = XENP014220 hIL2_0.89
APTSSSTKKTQLQLEHLLLDLQMILNGINNYKNPKLTRMLMKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 122 - protein, 133 aa, synthetic construct
  REGION 1..133: note = XENP014221 hIL2_0.90
APTSSSTKKTQLQLEHLLLDLQMILNGINNYKNPKLTRMLQKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 123 - protein, 133 aa, synthetic construct
  REGION 1..133: note = XENP014222 hIL2_0.91
APTSSSTKKTQLQLEHLLLDLQMILNGINNYKNPKLTQMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 124 - protein, 133 aa, synthetic construct
  REGION 1..133: note = XENP014223 hIL2_0.92
APTSSSTKKTQLQLEHLLLDLQMILNGINNYKNPKLTRMLIKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 125 - protein, 133 aa, synthetic construct
  REGION 1..133: note = XENP014224 hIL2_0.93
APTSSSTKKTQLQLEHLLLDLQMILNGINNYKNPKLTNMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 126 - protein, 133 aa, synthetic construct
  REGION 1..133: note = XENP014225 hIL2_0.94
APTSSSTKKTQLQLEHLLLDLQMILNGINNYKNPKLTRMLHKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 127 - protein, 133 aa, synthetic construct
  REGION 1..133: note = XENP014226 hIL2_0.95
APTSSSTKKTQLQLEHLLLDLQMILNGINNYKNPKLTQMLKYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 128 - protein, 133 aa, synthetic construct
  REGION 1..133: note = XENP014227 hIL2_0.96
APTSSSTKKTQLQLEHLLLDLQMILNGINNYKNPKLTQMLRYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 129 - protein, 133 aa, synthetic construct
  REGION 1..133: note = XENP014228 hIL2_0.97
APTSSSTKKTQLQLEHLLLDLQMILNGINNYKNPKLTQMLQYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 130 - protein, 133 aa, synthetic construct
  REGION 1..133: note = XENP014229 hIL2_0.98
APTSSSTKKTQLQLEHLLLDLQMILNGINNYKNPKLTQMLVYKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 131 - protein, 133 aa, synthetic construct
  REGION 1..133: note = XENP014230 hIL2_0.99
APTSSSTKKTQLQLEHLLLDLQMILNGINNYKNPKLTNMLKKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 132 - protein, 133 aa, synthetic construct
  REGION 1..133: note = XENP014231 hIL2_0.100
APTSSSTKKTQLQLEHLLLDLQMILNGINNYKNPKLTQMLHKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 133 - protein, 133 aa, synthetic construct
  REGION 1..133: note = XENP014232 hIL2_0.101
APTSSSTKKTQLQLEHLLLDLQMILNGINNYKNPKLTQMLKKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 134 - protein, 133 aa, synthetic construct
  REGION 1..133: note = XENP014233 hIL2_0.102
APTSSSTKKTQLQLEHLLLDLQMILNGINNYKNPKLTQMLQKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 135 - protein, 133 aa, synthetic construct
  REGION 1..133: note = XENP014234 hIL2_0.103
APTSSSTKKTQLQLEHLLLDLQMILNGINNYKNPKLTQMLVKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 136 - protein, 133 aa, synthetic construct
  REGION 1..133: note = XENP014235 hIL2_0.104
APTSSSTKKTQLQLEHLLLDLQMILNGINNYKNPKLTQMLRKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 137 - protein, 135 aa, synthetic construct
  REGION 1..135: note = XENP014277 hIL2_0.6
APTSSSTKKTQLQLEHLLLDLQMILNGISNHKNPRLARMLTFKFYMPEKATELKHLQCLEEELKPLEEALRLAPSKNFHLRPRDLISDVNVIVLELKGSETTFMCEYADETATIVEFLNRWITFSTSIISTLTGS

SEQ ID NO: 138 - protein, 135 aa, synthetic construct
  REGION 1..135: note = XENP014381 hIL2_0.202
APTSSSTKKTQLQLEHLLLDLQMILNGISNHKNPRLALMLTFKFYMPEKATELKHLQCLEEELKPLEEALRLAPSKNFHLRPRDLISDVNVIVLELKGSETTFMCEYADETATIVEFLNRWITFSTSIISTLTGS

SEQ ID NO: 139 - protein, 135 aa, synthetic construct; identical to SEQ ID NO: 40
  REGION 1..135: note = XENP014135 hIL2_0.1

SEQ ID NO: 140 - protein, 135 aa, synthetic construct; identical to SEQ ID NO: 47
  REGION 1..135: note = XENP014142 hIL2_0.15

SEQ ID NO: 141 - protein, 133 aa, synthetic construct; identical to SEQ ID NO: 48
  REGION 1..133: note = XENP014143 hIL2_0.16

SEQ ID NO: 142 - protein, 135 aa, synthetic construct; identical to SEQ ID NO: 49
  REGION 1..135: note = XENP014144 hIL2_0.17

SEQ ID NO: 143 - protein, 133 aa, synthetic construct; identical to SEQ ID NO: 73
  REGION 1..133: note = XENP014168 hIL2_0.41

SEQ ID NO: 144 - protein, 133 aa, synthetic construct; identical to SEQ ID NO: 94
  REGION 1..133: note = XENP014189 hIL2_0.62

SEQ ID NO: 145 - protein, 135 aa, synthetic construct
  REGION 1..135: note = XENP023816 hIL2_0.205
APTSSSTKKTELQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 146 - protein, 135 aa, synthetic construct
  REGION 1..135: note = XENP023817 hIL2_0.206
APTSSSTKKTQDQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 147 - protein, 135 aa, synthetic construct
  REGION 1..135: note = XENP023820 hIL2_0.209
APTSSSTKKTQLELEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 148 - protein, 135 aa, synthetic construct
  REGION 1..135: note = XENP023824 hIL2_0.213
APTSSSTKKTQLQLQHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 149 - protein, 135 aa, synthetic construct
  REGION 1..135: note = XENP023827 hIL2_0.216
APTSSSTKKTQLQLEYLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 150 - protein, 135 aa, synthetic construct
  REGION 1..135: note = XENP023830 hIL2_0.219
APTSSSTKKTQLQLEHLLDDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 151 - protein, 135 aa, synthetic construct
  REGION 1..135: note = XENP023833 hIL2_0.222
APTSSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 152 - protein, 135 aa, synthetic construct
  REGION 1..135: note = XENP023834 hIL2_0.223
APTSSSTKKTQLQLEHLLLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 153 - protein, 135 aa, synthetic construct
  REGION 1..135: note = XENP023836 hIL2_0.225
APTSSSTKKTQLQLEHLLLDLQMILNGINNYKNPRLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 154 - protein, 135 aa, synthetic construct
  REGION 1..135: note = XENP023837 hIL2_0.226
APTSSSTKKTQLQLEHLLLDLQMILNGINNYKNPKLSRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 155 - protein, 135 aa, synthetic construct
  REGION 1..135: note = XENP023838 hIL2_0.227
APTSSSTKKTQLQLEHLLLDLQMILNGINNYKNPKLTRMLTFRFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 156 - protein, 135 aa, synthetic construct
  REGION 1..135: note = XENP023839 hIL2_0.228
APTSSSTKKTQLQLEHLLLDLQMILNGINNYKNPKLTRMLTFKYYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 157 - protein, 135 aa, synthetic construct
  REGION 1..135: note = XENP023840 hIL2_0.229
APTSSSTKKTQLQLEHLLLDLQMILNGINNYKNPKLTRMLTFKFFMPKKATELKHLQCLEEELKPLEEVLNLAQSKNFHLRPRDLISNINVIVLELKGSETTFMCEYADETATIVEFLNRWITFSQSIISTLTGS

SEQ ID NO: 158 - protein, 135 aa, synthetic construct
  REGION 1..135: note = XENP023842 hIL2_0.231
APTSSSTKKTQLQLEHLLLDLQMILNGINNYKNPKLTRMLTFKFYMPRKATELKHLQCLEEELKPLEEVLNLAQSKNFHLRPRDLISNINVIVLELKGSETTFMCEYADETATIVEFLNRWITFSQSIISTLTGS

SEQ ID NO: 159 - protein, 135 aa, synthetic construct
  REGION 1..135: note = XENP023843 hIL2_0.232
APTSSSTKKTQLQLEHLLLDLQMILNGINNYKNPKLTRMLTFKFYMPKEATELKHLQCLEEELKPLEEVLNLAQSKNFHLRPRDLISNINVIVLELKGSETTFMCEYADETATIVEFLNRWITFSQSIISTLTGS

SEQ ID NO: 160 - protein, 135 aa, synthetic construct
  REGION 1..135: note = XENP023846 hIL2_0.235
APTSSSTKKTQLQLEHLLLDLQMILNGINNYKNPKLTRMLTFKFYMPKKATELKHLQCLEQELKPLEEVLNLAQSKNFHLRPRDLISNINVIVLELKGSETTFMCEYADETATIVEFLNRWITFSQSIISTLTGS

SEQ ID NO: 161 - protein, 135 aa, synthetic construct
  REGION 1..135: note = XENP023848 hIL2_0.237
APTSSSTKKTQLQLEHLLLDLQMILNGINNYKNPKLTRMLTFKFYMPKKATELKHLQCLEEQLKPLEEVLNLAQSKNFHLRPRDLISNINVIVLELKGSETTFMCEYADETATIVEFLNRWITFSQSIISTLTGS

SEQ ID NO: 162 - protein, 135 aa, synthetic construct
  REGION 1..135: note = XENP023849 hIL2_0.238
APTSSSTKKTQLQLEHLLLDLQMILNGINNYKNPKLTRMLTFKFYMPKKATELKHLQCLEEELRPLEEVLNLAQSKNFHLRPRDLISNINVIVLELKGSETTFMCEYADETATIVEFLNRWITFSQSIISTLTGS

SEQ ID NO: 163 - protein, 135 aa, synthetic construct
  REGION 1..135: note = XENP023851 hIL2_0.240
APTSSSTKKTQLQLEHLLLDLQMILNGINNYKNPKLTRMLTFKFYMPKKATELKHLQCLEEELKPLEQVLNLAQSKNFHLRPRDLISNINVIVLELKGSETTFMCEYADETATIVEFLNRWITFSQSIISTLTGS

SEQ ID NO: 164 - protein, 135 aa, synthetic construct
  REGION 1..135: note = XENP023852 hIL2_0.241
APTSSSTKKTQLQLEHLLLDLQMILNGINNYKNPKLTRMLTFKFYMPKKATELKHLQCLEEELKPLEELLNLAQSKNFHLRPRDLISNINVIVLELKGSETTFMCEYADETATIVEFLNRWITFSQSIISTLTGS

SEQ ID NO: 165 - protein, 135 aa, synthetic construct
  REGION 1..135: note = XENP023853 hIL2_0.242
APTSSSTKKTQLQLEHLLLDLQMILNGINNYKNPKLTRMLTFKFYMPKKATELKHLQCLEEELKPLEEVLNIAQSKNFHLRPRDLISNINVIVLELKGSETTFMCEYADETATIVEFLNRWITFSQSIISTLTGS

SEQ ID NO: 166 - protein, 135 aa, synthetic construct
  REGION 1..135: note = XENP023855 hIL2_0.244
APTSSSTKKTQLQLEHLLLDLQMILNGINNYKNPKLTRMLTFKFYMPKKATELKHLQCLEEELKPLEEVLNLAQSKNFHLDPRDLISNINVIVLELKGSETTFMCEYADETATIVEFLNRWITFSQSIISTLTGS

SEQ ID NO: 167 - protein, 135 aa, synthetic construct
  REGION 1..135: note = XENP023859 hIL2_0.248
APTSSSTKKTQLQLEHLLLDLQMILNGINNYKNPKLTRMLTFKFYMPKKATELKHLQCLEEELKPLEEVLNLAQSKNFHLRPRNLISNINVIVLELKGSETTFMCEYADETATIVEFLNRWITFSQSIISTLTGS

SEQ ID NO: 168 - protein, 135 aa, synthetic construct
  REGION 1..135: note = XENP023861 hIL2_0.250
APTSSSTKKTQLQLEHLLLDLQMILNGINNYKNPKLTRMLTFKFYMPKKATELKHLQCLEEELKPLEEVLNLAQSKNFHLRPRDLITNINVIVLELKGSETTFMCEYADETATIVEFLNRWITFSQSIISTLTGS

SEQ ID NO: 169 - protein, 135 aa, synthetic construct
  REGION 1..135: note = XENP023862 hIL2_0.251
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGS

SEQ ID NO: 170 - protein, 135 aa, synthetic construct
  REGION 1..135: note = XENP023868 hIL2_0.257
APTSSSTKKTQLQLEHLLLDLQMILNGINNYKNPKLTRMLTFKFYMPKKATELKHLQCLEEELKPLEEVLNLAQSKNFHLRPRDLISNINLIVLELKGSETTFMCEYADETATIVEFLNRWITFSQSIISTLTGS

SEQ ID NO: 171 - protein, 135 aa, synthetic construct
  REGION 1..135: note = XENP023869 hIL2_0.258
APTSSSTKKTQLQLEHLLLDLQMILNGINNYKNPKLTRMLTFKFYMPKKATELKHLQCLEEELKPLEEVLNLAQSKNFHLRPRDLISNINVLVLELKGSETTFMCEYADETATIVEFLNRWITFSQSIISTLTGS

SEQ ID NO: 172 - protein, 135 aa, synthetic construct
  REGION 1..135: note = XENP023871 hIL2_0.260
APTSSSTKKTQLQLEHLLLDLQMILNGINNYKNPKLTRMLTFKFYMPKKATELKHLQCLEEELKPLEEVLNLAQSKNFHLRPRDLISNINVIVLQLKGSETTFMCEYADETATIVEFLNRWITFSQSIISTLTGS

SEQ ID NO: 173 - protein, 135 aa, synthetic construct
  REGION 1..135: note = XENP023874 hIL2_0.263
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEFADETATIVEFLNRWITFSQSIISTLTGS

SEQ ID NO: 174 - protein, 135 aa, synthetic construct
  REGION 1..135: note = XENP023875 hIL2_0.264
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVRFLNRWITFSQSIISTLTGS

SEQ ID NO: 175 - protein, 135 aa, synthetic construct
  REGION 1..135: note = XENP023876 hIL2_0.265
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLDRWITFSQSIISTLTGS

SEQ ID NO: 176 - protein, 135 aa, synthetic construct
  REGION 1..135: note = XENP023878 hIL2_0.267
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNDWITFSQSIISTLTGS

SEQ ID NO: 177 - protein, 135 aa, synthetic construct
  REGION 1..135: note = XENP023882 hIL2_0.271
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWISFSQSIISTLTGS

SEQ ID NO: 178 - protein, 135 aa, synthetic construct
  REGION 1..135: note = XENP023883 hIL2_0.272
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSESIISTLTGS

SEQ ID NO: 179 - protein, 135 aa, synthetic construct
  REGION 1..135: note = XENP023885 hIL2_0.274
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQTIISTLTGS

SEQ ID NO: 180 - protein, 135 aa, synthetic construct
  REGION 1..135: note = XENP023886 hIL2_0.275
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSILSTLTGS

SEQ ID NO: 181 - protein, 135 aa, synthetic construct
  REGION 1..135: note = XENP023888 hIL2_0.277
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIITTLTGS

SEQ ID NO: 182 - protein, 135 aa, synthetic construct
  REGION 1..135: note = XENP023889 hIL2_0.278
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLSGS

SEQ ID NO: 183 - protein, 231 aa, synthetic construct; identical to SEQ ID NO: 10
  REGION 1..231: note = Figure 20

SEQ ID NO: 184 - protein, 364 aa, synthetic construct
  REGION 1..364: note = Figure 20
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 231 aa, synthetic construct; identical to SEQ ID NO: 10
  REGION 1..231: note = Figure 20

SEQ ID NO: 186 - protein, 364 aa, synthetic construct
  REGION 1..364: note = Figure 20
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 231 aa, synthetic construct; identical to SEQ ID NO: 10
  REGION 1..231: note = Figure 20

SEQ ID NO: 188 - protein, 364 aa, synthetic construct
  REGION 1..364: note = Figure 20
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 231 aa, synthetic construct; identical to SEQ ID NO: 10
  REGION 1..231: note = Figure 20

SEQ ID NO: 190 - protein, 364 aa, synthetic construct
  REGION 1..364: note = Figure 20
APTSSSTKKTQLQLEHLLLDLQMILNGISNHKNPRLARMLTFKFYMPEKATELKHLQCLEEELKPLEEALRLAPSKNFHLRPRDLISDVNVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 231 aa, synthetic construct; identical to SEQ ID NO: 10
  REGION 1..231: note = Figure 20

SEQ ID NO: 192 - protein, 364 aa, synthetic construct
  REGION 1..364: note = Figure 20
APTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSTISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 231 aa, synthetic construct; identical to SEQ ID NO: 10
  REGION 1..231: note = Figure 20

SEQ ID NO: 194 - protein, 364 aa, synthetic construct
  REGION 1..364: note = Figure 20
APTSSSTKKTQLQLEHLLLDLQMILNGISNHKNPRLARMLTFKFYMPEKATELKHLQCLEEELKPLEEALRLAPSKNFHLRPRDLISDVNVIVLELKGSETTFMCEYADETATIVEFLNRWITFSTSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 231 aa, synthetic construct; identical to SEQ ID NO: 10
  REGION 1..231: note = Figure 20

SEQ ID NO: 196 - protein, 364 aa, synthetic construct
  REGION 1..364: note = Figure 20
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSTSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 231 aa, synthetic construct; identical to SEQ ID NO: 10
  REGION 1..231: note = Figure 20

SEQ ID NO: 198 - protein, 364 aa, synthetic construct
  REGION 1..364: note = Figure 20
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 231 aa, synthetic construct; identical to SEQ ID NO: 10
  REGION 1..231: note = Figure 20

SEQ ID NO: 200 - protein, 364 aa, synthetic construct
  REGION 1..364: note = Figure 20
APTSSSTKKTQLQLEHLLLDLQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 231 aa, synthetic construct; identical to SEQ ID NO: 10
  REGION 1..231: note = Figure 20

SEQ ID NO: 202 - protein, 364 aa, synthetic construct
  REGION 1..364: note = Figure 20
APTSSSTKKTQLQLEHLLLDLQMILNGINNYKNPKLTRMLTFKFYMPKKATELKHLQCLEEELKPLEEVLNLAQSKNFHFDPRDVISNINVFVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 231 aa, synthetic construct; identical to SEQ ID NO: 10
  REGION 1..231: note = Figure 20

SEQ ID NO: 204 - protein, 364 aa, synthetic construct
  REGION 1..364: note = Figure 20
APTSSSTKKTQLQLEHLRLDLEMILNGINNYKNPKLTRMLTFKFYMPKKATELKHLQCLEEELKPLEEVLNLAQSKNFHFDPRDVISNINVFVLELKGSETTFMCEYADETATIVEFLNRWITFSTSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 231 aa, synthetic construct; identical to SEQ ID NO: 10
  REGION 1..231: note = Figure 20

SEQ ID NO: 206 - protein, 364 aa, synthetic construct
  REGION 1..364: note = Figure 20
APTSSSTKKTQLQLEHLRLDLEMILNGINNYKNPKLTRMLTFKFYMPKKATELKHLQCLEEELKPLEEVLNLAQSKNFHFDPRDVISNINVFVLELKGSETTFMCEYADETATIVEFLNRWITFSTSIIRTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 231 aa, synthetic construct; identical to SEQ ID NO: 10
  REGION 1..231: note = Figure 20

SEQ ID NO: 208 - protein, 364 aa, synthetic construct
  REGION 1..364: note = Figure 20
APASSSTKKTQLQLEHLLLDLQMILNGINNYKNPKLTRMLTAKFAMPKKATELKHLQCLEEELKPLEEVLNGAQSKNFHLRPRDLISRINVIVLELKGSETTFMCEYADETATIVEFLNRWITFAQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 231 aa, synthetic construct; identical to SEQ ID NO: 10
  REGION 1..231: note = Figure 20

SEQ ID NO: 210 - protein, 364 aa, synthetic construct
  REGION 1..364: note = Figure 20
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 231 aa, synthetic construct; identical to SEQ ID NO: 10
  REGION 1..231: note = Figure 20

SEQ ID NO: 212 - protein, 364 aa, synthetic construct
  REGION 1..364: note = Figure 20
APASSSTKKTQLQLEHLLLDLQMILNGINNYKNPKLTRMLTAKFAMPKKATELKHLQCLEEELKPLEEVLNGAQSKNFHLRPRDLISRINVIVLELKGSETTFMCEYADETATIVEFLNRWITFATSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 364 aa, synthetic construct
  REGION 1..364: note = Figure 23
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 364 aa, synthetic construct
  REGION 1..364: note = Figure 23
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 384 aa, synthetic construct
  REGION 1..384: note = Figure 25
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 384 aa, synthetic construct
  REGION 1..384: note = Figure 25
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 231 aa, synthetic construct; identical to SEQ ID NO: 10
  REGION 1..231: note = Figure 25

SEQ ID NO: 218 - protein, 384 aa, synthetic construct
  REGION 1..384: note = Figure 25
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 231 aa, synthetic construct; identical to SEQ ID NO: 10
  REGION 1..231: note = Figure 25

SEQ ID NO: 220 - protein, 384 aa, synthetic construct
  REGION 1..384: note = Figure 25
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 231 aa, synthetic construct; identical to SEQ ID NO: 10
  REGION 1..231: note = Figure 25

SEQ ID NO: 222 - protein, 384 aa, synthetic construct
  REGION 1..384: note = Figure 25
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 231 aa, synthetic construct; identical to SEQ ID NO: 10
  REGION 1..231: note = Figure 27

SEQ ID NO: 224 - protein, 364 aa, synthetic construct
  REGION 1..364: note = Figure 27
APASSSTKKTQLQLEHLLDDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 231 aa, synthetic construct; identical to SEQ ID NO: 10
  REGION 1..231: note = Figure 27

SEQ ID NO: 226 - protein, 364 aa, synthetic construct
  REGION 1..364: note = Figure 27
APASSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 231 aa, synthetic construct; identical to SEQ ID NO: 10
  REGION 1..231: note = Figure 27

SEQ ID NO: 228 - protein, 364 aa, synthetic construct
  REGION 1..364: note = Figure 27
APASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 231 aa, synthetic construct; identical to SEQ ID NO: 10
  REGION 1..231: note = Figure 27

SEQ ID NO: 230 - protein, 364 aa, synthetic construct
  REGION 1..364: note = Figure 27
APASSSTKKTQLQLEHLLLDLQMILNGINNYKNPKLTRMLTFKFYMPKKATELKHLQCLEEELKPLEEVLNLAQSKNFHLRPRDLISKINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 231 aa, synthetic construct; identical to SEQ ID NO: 10
  REGION 1..231: note = Figure 27

SEQ ID NO: 232 - protein, 364 aa, synthetic construct
  REGION 1..364: note = Figure 27
APTSSSTKKTQLQLEHLLLDLQMILNGISNHKNPRLARMLTFKFYMPEKATELKHLQCLEEELKPLEEVLRLAQSKNFHLRPRDLISDVNVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 231 aa, synthetic construct; identical to SEQ ID NO: 10
  REGION 1..231: note = Figure 27

SEQ ID NO: 234 - protein, 364 aa, synthetic construct
  REGION 1..364: note = Figure 27
APTSSSTKKTQLQLEHLLDDLQMILNGISNHKNPRLARMLTFKFYMPEKATELKHLQCLEEELKPLEEVLRLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 231 aa, synthetic construct; identical to SEQ ID NO: 10
  REGION 1..231: note = Figure 27

SEQ ID NO: 236 - protein, 364 aa, synthetic construct
  REGION 1..364: note = Figure 27
APTSSSTKKTQLQLEHLLLNLQMILNGISNHKNPRLARMLTFKFYMPEKATELKHLQCLEEELKPLEEVLRLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 231 aa, synthetic construct; identical to SEQ ID NO: 10
  REGION 1..231: note = Figure 27

SEQ ID NO: 238 - protein, 364 aa, synthetic construct
  REGION 1..364: note = Figure 27
APTSSSTKKTQLQLEHLLDDLQMILNGISNHKNPRLARMLTFKFYMPEKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 231 aa, synthetic construct; identical to SEQ ID NO: 10
  REGION 1..231: note = Figure 27

SEQ ID NO: 240 - protein, 364 aa, synthetic construct
  REGION 1..364: note = Figure 27
APTSSSTKKTQLQLEHLLLNLQMILNGISNHKNPRLARMLTFKFYMPEKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 231 aa, synthetic construct; identical to SEQ ID NO: 10
  REGION 1..231: note = Figure 27

SEQ ID NO: 242 - protein, 364 aa, synthetic construct
  REGION 1..364: note = Figure 27
APASSSTKKTQLQLEHLLDDLQMILNGINNYKNPRLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 231 aa, synthetic construct; identical to SEQ ID NO: 10
  REGION 1..231: note = Figure 27

SEQ ID NO: 244 - protein, 364 aa, synthetic construct
  REGION 1..364: note = Figure 27
APASSSTKKTQLQLEHLLDDLQMILNGINNYKNPKLRRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 231 aa, synthetic construct; identical to SEQ ID NO: 10
  REGION 1..231: note = Figure 27

SEQ ID NO: 246 - protein, 364 aa, synthetic construct
  REGION 1..364: note = Figure 27
APASSSTKKTQLQLEHLLLNLQMILNGINNYKNPKLRRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 231 aa, synthetic construct; identical to SEQ ID NO: 10
  REGION 1..231: note = Figure 27

SEQ ID NO: 248 - protein, 364 aa, synthetic construct
  REGION 1..364: note = Figure 27
APASSSTKKTQLQLEHLLDDLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 231 aa, synthetic construct; identical to SEQ ID NO: 10
  REGION 1..231: note = Figure 27

SEQ ID NO: 250 - protein, 364 aa, synthetic construct
  REGION 1..364: note = Figure 27
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 231 aa, synthetic construct; identical to SEQ ID NO: 10
  REGION 1..231: note = Figure 30

SEQ ID NO: 252 - protein, 364 aa, synthetic construct
  REGION 1..364: note = Figure 30
APASSSTKKTQLQLEHLLLNLQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 231 aa, synthetic construct; identical to SEQ ID NO: 10
  REGION 1..231: note = Figure 30

SEQ ID NO: 254 - protein, 364 aa, synthetic construct
  REGION 1..364: note = Figure 30
APASSSTKKTQLQLEHLLLNLQMILNGINNYKNPKLRIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 231 aa, synthetic construct; identical to SEQ ID NO: 10
  REGION 1..231: note = Figure 30

SEQ ID NO: 256 - protein, 364 aa, synthetic construct
  REGION 1..364: note = Figure 30
APASSSTKKTQLQLEHLLLNLQMILNGINNYKNPKLTIMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 231 aa, synthetic construct; identical to SEQ ID NO: 10
  REGION 1..231: note = Figure 30

SEQ ID NO: 258 - protein, 374 aa, synthetic construct
  REGION 1..374: note = Figure 30
APASSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 231 aa, synthetic construct; identical to SEQ ID NO: 10
  REGION 1..231: note = Figure 30

SEQ ID NO: 260 - protein, 374 aa, synthetic construct
  REGION 1..374: note = Figure 30
APASSSTKKTQLQLEHLLLNLQMILNGINNYKNPKLRRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 231 aa, synthetic construct; identical to SEQ ID NO: 10
  REGION 1..231: note = Figure 30

SEQ ID NO: 262 - protein, 374 aa, synthetic construct
  REGION 1..374: note = Figure 30
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 231 aa, synthetic construct; identical to SEQ ID NO: 10
  REGION 1..231: note = Figure 30

SEQ ID NO: 264 - protein, 374 aa, synthetic construct
  REGION 1..374: note = Figure 30
APASSSTKKTQLQLEHLLLNLQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 231 aa, synthetic construct; identical to SEQ ID NO: 10
  REGION 1..231: note = Figure 30

SEQ ID NO: 266 - protein, 374 aa, synthetic construct
  REGION 1..374: note = Figure 30
APASSSTKKTQLQLEHLLLNLQMILNGINNYKNPKLRIMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 231 aa, synthetic construct; identical to SEQ ID NO: 10
  REGION 1..231: note = Figure 30

SEQ ID NO: 268 - protein, 374 aa, synthetic construct
  REGION 1..374: note = Figure 30
APASSSTKKTQLQLEHLLLNLQMILNGINNYKNPKLTIMLTFKFYMPKKATELKHLQCLEEELKPLEEVLKLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 269 - protein, 364 aa, synthetic construct
  REGION 1..364: note = Figure 30
APASSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 364 aa, synthetic construct
  REGION 1..364: note = Figure 30
APASSSTKKTQLQLEHLLLNLQMILNGINNYKNPKLRRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 364 aa, synthetic construct
  REGION 1..364: note = Figure 30
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 364 aa, synthetic construct
  REGION 1..364: note = Figure 30
APASSSTKKTQLQLEHLLLNLQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 364 aa, synthetic construct
  REGION 1..364: note = Figure 30
APASSSTKKTQLQLEHLLLNLQMILNGINNYKNPKLRIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 364 aa, synthetic construct
  REGION 1..364: note = Figure 30
APASSSTKKTQLQLEHLLLNLQMILNGINNYKNPKLTIMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 374 aa, synthetic construct
  REGION 1..374: note = Figure 30
APASSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 374 aa, synthetic construct
  REGION 1..374: note = Figure 30
APASSSTKKTQLQLEHLLLNLQMILNGINNYKNPKLRRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 374 aa, synthetic construct
  REGION 1..374: note = Figure 30
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 374 aa, synthetic construct
  REGION 1..374: note = Figure 30
APASSSTKKTQLQLEHLLLNLQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 374 aa, synthetic construct
  REGION 1..374: note = Figure 30
APASSSTKKTQLQLEHLLLNLQMILNGINNYKNPKLRIMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 374 aa, synthetic construct
  REGION 1..374: note = Figure 30
APASSSTKKTQLQLEHLLLNLQMILNGINNYKNPKLTIMLTFKFYMPKKATELKHLQCLEEELKPLEEVLKLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 364 aa, synthetic construct
  REGION 1..364: note = Figure 34
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFAQSIISTLT

SEQ ID NO: 282 - protein, 364 aa, synthetic construct
  REGION 1..364: note = Figure 43
APASSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 283 - protein, 364 aa, synthetic construct
  REGION 1..364: note = Figure 43
APASSSTKKTQLQLEHLLLNLQMILNGINNYKNPKLRRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 284 - protein, 364 aa, synthetic construct
  REGION 1..364: note = Figure 43
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 285 - protein, 364 aa, synthetic construct
  REGION 1..364: note = Figure 43
APASSSTKKTQLQLEHLLLNLQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 286 - protein, 364 aa, synthetic construct
  REGION 1..364: note = Figure 43
APASSSTKKTQLQLEHLLLNLQMILNGINNYKNPKLRIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 287 - protein, 364 aa, synthetic construct
  REGION 1..364: note = Figure 43
APASSSTKKTQLQLEHLLLNLQMILNGINNYKNPKLTIMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 288 - protein, 364 aa, synthetic construct
  REGION 1..364: note = Figure 43
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 289 - protein, 364 aa, synthetic construct
  REGION 1..364: note = Figure 43
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 290 - protein, 374 aa, synthetic construct
  REGION 1..374: note = Figure 43
APASSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 291 - protein, 374 aa, synthetic construct
  REGION 1..374: note = Figure 43
APASSSTKKTQLQLEHLLLNLQMILNGINNYKNPKLRRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 292 - protein, 374 aa, synthetic construct
  REGION 1..374: note = Figure 43
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTGGGGSGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 293 - protein, 231 aa, synthetic construct; identical to SEQ ID NO: 30
  REGION 1..231: note = Figure 44

SEQ ID NO: 294 - protein, 364 aa, synthetic construct
  REGION 1..364: note = Figure 44
APASSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 295 - protein, 231 aa, synthetic construct; identical to SEQ ID NO: 30
  REGION 1..231: note = Figure 44

SEQ ID NO: 296 - protein, 364 aa, synthetic construct
  REGION 1..364: note = Figure 44
APASSSTKKTQLQLEHLLLNLQMILNGINNYKNPKLRRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 297 - protein, 231 aa, synthetic construct; identical to SEQ ID NO: 30
  REGION 1..231: note = Figure 44

SEQ ID NO: 298 - protein, 364 aa, synthetic construct
  REGION 1..364: note = Figure 44
APASSSTKKTQLQLEHLLLNLQMILNGINNYKNPKLTRMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 299 - protein, 231 aa, synthetic construct; identical to SEQ ID NO: 30
  REGION 1..231: note = Figure 44

SEQ ID NO: 300 - protein, 364 aa, synthetic construct
  REGION 1..364: note = Figure 44
APASSSTKKTQLQLEHLLLNLQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 301 - protein, 231 aa, synthetic construct; identical to SEQ ID NO: 30
  REGION 1..231: note = Figure 44

SEQ ID NO: 302 - protein, 364 aa, synthetic construct
  REGION 1..364: note = Figure 44
APASSSTKKTQLQLEHLLLNLQMILNGINNYKNPKLRIMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 303 - protein, 231 aa, synthetic construct; identical to SEQ ID NO: 30
  REGION 1..231: note = Figure 44

SEQ ID NO: 304 - protein, 364 aa, synthetic construct
  REGION 1..364: note = Figure 44
APASSSTKKTQLQLEHLLLNLQMILNGINNYKNPKLTIMLTFKFYMPKKATELKHLQCLEEELKPLEEVLKLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 305 - protein, 231 aa, synthetic construct; identical to SEQ ID NO: 30
  REGION 1..231: note = Figure 44

SEQ ID NO: 306 - protein, 364 aa, synthetic construct
  REGION 1..364: note = Figure 44
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 307 - protein, 231 aa, synthetic construct; identical to SEQ ID NO: 30
  REGION 1..231: note = Figure 44

SEQ ID NO: 308 - protein, 364 aa, synthetic construct
  REGION 1..364: note = Figure 44
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

(19) United States
(12) Patent Application Publication
(10) Pub. No.: US 2025/0092108 A1
(43) Pub. Date: Mar. 20, 2025

(54) ENGINEERED IL-2 Fc FUSION PROTEINS

(71) Applicant: Xencor, Inc., Pasadena, CA (US)

(72) Inventors: Matthew Bernett, Monrovia, CA (US); John Desjarlais, Pasadena, CA (US); Rajat Varma, Monrovia, CA (US); Rumana Rashid, Temple City, CA (US)

(21) Appl. No.: 18/628,181

(22) Filed: Apr. 5, 2024

Related U.S. Application Data
(63) Continuation of application No. 17/578,265, filed on Jan. 18, 2022, now Pat. No. 11,981,717.
Application No. 17/578,265 is a continuation of application No. 16/206,849, filed on Nov. 30, 2018, now Pat. No. 11,319,355.
(60) Provisional application No. 62/607,850, filed on Dec. 19, 2017.
(60) Provisional application No. 62/675,070, filed on May 22, 2018.

(51) Int. Cl. C07K 14/55; A61K 38/00; C07K 14/735; C12N 15/85

(52) U.S. Cl. CPC C07K 14/55; C07K 14/70535; C12N 15/85; A61K 38/00; C07K 2319/30

(57) ABSTRACT

The present invention provides IL-2-Fc fusion proteins comprising IL-2 variants.

### I. PRIORITY CLAIM

This application is a continuation of U.S. patent application Ser. No. 17/578,265, filed Jan. 18, 2022, which is a continuation of U.S. patent application Ser. No. 16/206,849, filed Nov. 30, 2018, which claims the benefit of U.S. Provisional Application Nos. 62/607,850, filed Dec. 19, 2017 and 62/675,070, filed May 22, 2018, which are expressly incorporated herein by reference in their entireties, with particular reference to the figures, legends, and claims therein.

### II. SEQUENCE LISTING

The instant application contains a Sequence Listing which has been submitted electronically in XML format and is hereby incorporated by reference in its entirety. Said XML copy, created on Apr. 5, 2024, is named 067461-5217-US02.xml and is 448,535 bytes in size.

### III. BACKGROUND OF THE INVENTION

Immune system homeostasis relies on a fine balance between a variety of immune cell populations, including CD8+ and CD4+ T cells (CD3+CD25−FOXP3−) and regulatory T cells (Tregs; CD3+CD4+CD25+FOXP3+). Disruptions to this balance can lead to diseases such as autoimmune diseases wherein T cells remain unregulated and attack the body's own tissues. In a normal state, Tregs modulate T cell differentiation and effector and cytotoxic functions. Therefore, a major premise in this regard is that defects in Treg cell number and/or function are a contributing factor to disease states. As such, the ability to alter the balance between cytotoxicity and regulation by fine-tuning the T cell response has great potential for the treatment of autoimmune and other diseases.

IL-2 functions in aiding the proliferation and differentiation of B cells, T cells and NK cells. IL-2 is also essential for Treg function and survival. IL-2 exerts its cell signaling function through binding to a high-affinity trimeric receptor complex consisting of three distinct proteins: a common gamma chain (γc; CD132) and IL-2 receptor B-chain (IL-2Rβ; CD122), which are shared with IL-15, as well as a unique alpha chain receptor (IL-2Rγ; CD25). IL-2 can also exert its cell signaling function through binding to an intermediate-affinity dimeric receptor complex consisting only of IL-2Rβ and γc (IL-2Rβγ).

Due to the low concentrations of IL-2 that typically exists in tissues, IL-2 preferentially activates cells that express the high affinity receptor complex (CD25:CD122:CD132; IL-2Rγβγ), and therefore favors FOXP3+ Tregs which constitutively express CD25. However, IL-2 can also activate and induce proliferation of FOXP3− T cells that express the intermediate affinity receptor complex (CD122:CD132; IL-2Rβγ). FOXP3− T cells, such as CD4+ or CD8+ T cells, may contribute to inflammation, autoimmunity, organ graft rejection, or graft-versus-host disease. Because of the potential of IL-2 to promote or reduce both T cells and Tregs with limited selectivity, there is a strong need in the field to create more selective Treg modulators. Additionally, as a potential drug, IL-2 suffers from a very fast clearance, with half-life measured in minutes, which hinders favorable dosing. The present invention solves both these problems by providing novel IL-2-Fc fusion proteins.

Accordingly, there is a need to provide useful IL-2 variants and Fc fusion proteins.

### IV. SUMMARY OF THE INVENTION

Accordingly, in some aspects, the disclosure provides compositions comprising a variant human IL-2 protein (as compared to SEQ ID NO:2) wherein said variant IL-2 protein comprises amino acid substitution(s) selected from the group of T3A, R38A; R38D; R38E; R38F; R38G; R38H; R38I; R38K; R38L; R38M; R38N; R38P; R38Q; R38S; R38T; R38V; R38W; R38Y; T41A; T41D; T41E; T41F; T41G; T41H; T41I; T41K; T41L; T41M; T41N; T41P; T41Q; T41R; T41S; T41V; T41W; T41Y; F42A; F42D; F42E; F42G; F42H; F42I; F42K; F42L; F42M; F42N; F42P; F42Q; F42R; F42S; F42T; F42V; F42W; F42Y; R38Q/T41K; R38Q/T41Q; R38E/T41K; R38Q/T41R; R38N/T41Q; R38Q/T41V; R38N/T41V; R38Q/T41M; R38Q/T41S; R38Q/T41L; R38N/T41M; T41I/F42Y; T41E/F42Y′ T41D/F42Y; T41M/F42Y; 41Q/F42Y; T41E/F42H; T41E/F42L; T41E/F42P; R38Q/F42Y; R38N/T41R; R38N/T41K; R38V/T41R; R38P/T41R; T41E/F42K; T41D/F42K; T41M/F42K; T41Q/F42K; R38Q/F42K; T41I/F42K; R38N/F42K; T41H/F42K; R38Q/T41K/F42Y; R38Q/T41R/F42Y; R38Q/T41Q/F42Y; R38Q/T41V/F42Y; R38N/T41K/F42K; R38Q/T41H/F42K; R38Q/T41K/F42K; R38Q/T41Q/F42K; 38Q/T41V/F42K; R38Q/T41R/F42K; Q11E; L12D; Q13E; E15Q; H16Y; L19D; D20N; N29S/Y31H/K35R/T37A/R38L/K48E/V69A/N71R/Q74P/N88D/I89V/Q126T; Q22E; K35R; T37S; K43R; F44Y; Y45F; K48R; K49E; E61Q; E62Q; K64R; E68Q; V69L; L72I; R81D; D84N; S87T; N88D; V91L; I92L; E95Q; Y107F; E116R; N119D; R120D; T123S; C125S/Q126E; C125S/S127T; C125S/I129L; C125S/S130T; C125S/T133S; T3A; F42A/Y45A/L72G; N29S/Y31H/K35R/T37A/K48E/V69A/N71R/Q74P/N88D/I89V; V69A/Q74P/I128T; N29S/Y31H/K35R/T37A/K48E/V69A/N71R/Q74P/N88D/I89V/Q126T; C125S/Q126T; N88R; R38I; L80F/R81D/L85V/I92F; L18R/L80F/R81D/L85V/I92F/Q126T; L18R/L80F/R81D/L85V/I92F/Q126T/S130R; F42A/Y45A/L72G/N88R; F42A/Y45A/L72G/Q126T; F42A/Y45A/L72G/N88R/Q126T; L19D; D20N; N88D; N88K; N88R; N88R; N88R; F42A/Y45A/L72G; N29S/Y31H/K35R/T37A/K48E/N71R/N88D/I89V; L19D/N29S/Y31H/K35R/T37A/K48E/N71R; D20N/N29S/Y31H/K35R/T37A; K48E/N71R; L19D/N29S/Y31H/K35R/T37A/K48E; D20N/N29S/Y31H/K35R/T37A; K48E; L19D K35R; L19D/T37R; D20N/T37R; L19D/N71K; D20N/N71K; D20N/R38I; D20N/T37R; 38I; D20N/R38I/N71K; D20N/N71K; D20N; D20N/T37R; D20N/R38I; D20N/T37R R38I; D20N/R38I/N71K; D20N; D20N/T37R; D20N/N71K; D20N/R38I; D20N/T37R R38I; D20N/R38I/N71K; D20N; D20N/T37R; D20N/N71K; D20N/R38I; D20N/T37R/R38I; D20N/R38I/N71K; N29S/Y31H/K35R/T37A/K48E/V69A/N71R/Q74P/N88D/189V/Q126T, R38A/C125S; R38D/C125S; R38E/C125S; R38F/C125S; R38G/C125S; R38H/C125S; R38I/C125S; R38K/C125S; R38L/C125S; R38M/C125S; R38N/C125S; R38P/C125S; R38Q/C125S; R38S/C125S; R38T/C125S; R38V/C125S; R38W/C125S; R38Y/C125S; T41A/C125S; T41D/C125S; T41E/C125S; T41F/C125S; T41G/C125S; T41H/C125S; T41I/C125S; T41K/C125S; T41L/C125S; T41M/C125S; T41N/C125S; T41P/C125S; T41Q/C125S; T41R/C125S; T41S/C125S; T41V/C125S; T41W/C125S; T41Y/C125S; F42A/C125S; F42D/C125S; F42E/C125S; F42G/C125S; F42H/C125S; F42I/C125S; F42K/C125S; F42L/C125S; F42M/C125S; F42N/C125S; F42P/C125S; F42Q/C125S; F42R/C125S; F42S/C125S; F42T/C125S; F42V/C125S; F42W/C125S; F42Y/C125S; R38Q/T41K/C125S; R38Q/T41Q/C125S; R38E/T41K/C125S; R38Q/T41R/C125S; R38N/T41Q/C125S; R38Q/T41V/C125S; R38N/T41V/C125S; R38Q/T41M/C125S; R38Q/T41S/C125S; R38Q/T41L/C125S; R38N/T41M/C125S; T41I/F42Y/C125S; T41E/F42Y/C125S′ T41D/F42Y/C125S; T41M/F42Y/C125S; 41Q/F42Y/C125S; T41E/F42H/C125S; T41E/F42L/C125S; T41E/F42P/C125S; R38Q/F42Y/C125S; R38N/T41R/C125S; R38N/T41K/C125S; R38V/T41R/C125S; R38P/T41R/C125S; T41E/F42K/C125S; T41D/F42K/C125S; T41M/F42K/C125S; T41Q/F42K/C125S; R38Q/F42K/C125S; T41I/F42K/C125S; R38N/F42K/C125S; T41H/F42K/C125S; R38Q/T41K/F42Y/C125S; R38Q/T41R/F42Y/C125S; R38Q/T41Q/F42Y/C125S; R38Q/T41V/F42Y/C125S; R38N/T41K/F42K/C125S; R38Q/T41H/F42K/C125S; R38Q/T41K/F42K/C125S; R38Q/T41Q/F42K/C125S; 38Q/T41V/F42K/C125S; R38Q/T41R/F42K/C125S; N29S/Y31H/K35R/T37A/R38L/K48E/V69A/N71R/Q74P/N88D/189V/C125S/Q126T; Q11E/C125S; L12D/C125S; Q13E/C125S; E15Q/C125S; H16Y/C125S; L19D/C125S; D20N/C125S; Q22E/C125S; K35R/C125S; T37S/C125S; K43R/C125S; F44Y/C125S; Y45F/C125S; K48R/C125S; K49E/C125S; E61Q/C125S; E62Q/C125S; K64R/C125S; E68Q/C125S; V69L/C125S; L72I/C125S; R81D/C125S; D84N/C125S; S87T/C125S; N88D/C125S; V91L/C125S; 192L/C125S; E95Q/C125S; Y107F/C125S; E116R/C125S; N119D/C125S; R120D/C125S; T123S/C125S; C125S/Q126E; C125S/S127T; C125S/1129L; C125S/S130T; C125S/T133S; T3A/C125S; T3A/F42A/Y45A/L72G/C125A; N29S/Y31H/K35R/T37A/K48E/V69A/N71R/Q74P/N88D/189V/C125S; V69A/Q74P/I128T/C125S; N29S/Y31H/K35R/T37A/K48E/V69A/N71R/Q74P/N88D/189V/C125S/Q126T; C125S/Q126T; N88R/C125S; R38I/C125S; L80F/R81D/L85V/192F/C125S; L18R/L80F/R81D/L85V/I92F/C125S/Q126T; L18R/L80F/R81D/L85V/I92F/C125S/Q126T/S130R; T3A/F42A/Y45A/L72G/N88R/C125A; T3A/F42A/Y45A/L72G/C125A/Q126T; T3A/F42A/Y45A/L72G/N88R/C125A/Q126T; T3A/L19D/C125S; T3A/D20N/C125S; T3A/N88D/C125S; T3A/N88K/C125S; N88R/C125S; N88R/C125S; N88R/C125S; T3A/F42A/Y45A/L72G/C125A; N29S/Y31H/K35R/T37A/K48E/N71R/N88D/I89V/C125S; L19D/N29S/Y31H/K35R/T37A/K48E/N71R/C125S; D20N/N29S/Y31H/K35R/T37A; K48E/N71R/C125S; L19D/N29S/Y31H/K35R/T37A/K48E/C125S; D20N/N29S/Y31H/K35R/T37A; K48E/C125S; T3A/L19D K35R/C125S; T3A/L19D/T37R/C125S; T3A/D20N/T37R/C125S; T3A/L19D/N71K/C125S; T3A/D20N/N71K/C125S; T3A/D20N/R38I/C125S; T3A/D20N/T37R; 38I/C125S; T3A/D20N/R38I/N71K/C125S; T3A/D20N/N71K/C125S; T3A/D20N/C125S; T3A/D20N/T37R/C125S; T3A/D20N/R38I/C125S; T3A/D20N/T37R R38I/C125S; T3A/D20N/R38I/N71K/C125S; T3A/D20N/C125S; T3A/D20N/T37R/C125S; T3A/D20N/N71K/C125S; T3A/D20N/R38I/C125S; T3A/D20N/T37R R38I/C125S; T3A/D20N/R38I/N71K/C125S; T3A/D20N/C125S; T3A/D20N/T37R/C125S; T3A/D20N/N71K/C125S; T3A/D20N/R38I/C125S; T3A/D20N/T37R/R38I/C125S; T3A/D20N/R38I/N71K/C125S; and N29S/Y31H/K35R/T37A/K48E/V69A/N71R/Q74P/N88D/I89V/C125S/Q126T.

In further aspects, the disclosure provides IL-2-Fc fusion dimeric proteins comprising: a) a first monomer comprising, from N- to C-terminal: i) a variant IL-2 protein according to any of the claims; ii) a first domain linker; and iii) a first variant Fc domain; and b) a second monomer comprising a second variant Fc domain.

In additional aspects, the invention provides IL-2-Fc fusion dimeric proteins wherein said second monomer comprises, from N- to C-terminal: a) a variant IL-2 protein according to any of the claims; b) a second domain linker; and c) said second variant Fc domain.

In further aspects the IL-2-Fc fusion dimeric proteins have first and second variant Fe domains contain heterodimerization variants selected from the group consisting of L368D/K370S:S364K/E357Q; L368D/K370S:S364K; L368E/K370S:S364K;/T41I E/K360E/Q362E:D401K; and T366S/L368A/Y407V:T366W. In some cases, the IL-2-Fc fusion proteins further comprise ablation variants, including P233P/L234V/L235A/G236_/S267K. In some aspects the fusion proteins also comprise Fc domains with amino acid substitutions M428L/N434S or M428L/N434A. In some aspects, the domain linker is the IGGI hinge, and in others it can include a linker selected from (GS)n, (GSGGS)n, (GGGGS)n, and (GGGS)n, where n is an integer of at least one.

In additional aspects, the invention comprises a polypeptide composition comprising a variant human IL-2 protein wherein said variant IL-2 protein comprises amino acid substitution(s) as compared to SEQ ID NO:2 selected from the group of T3A/D20N/T37R and T3A/D20N/N71K. In some cases, the variant IL-2 protein further comprises a C125S variant or a C125A variant.

In a further aspect, the polypeptide composition is a homodimeric protein complex wherein each protein monomer comprises said variant IL-2 protein covalently attached to an Fc domain. In some aspects, each of said Fc domains is a variant Fc domain.

In a further aspect, the polypeptide composition is a heterodimeric protein complex comprising a first protein monomer comprising said variant IL-2 protein is covalently attached to a first variant Fc domain and a second protein monomer comprising a second variant Fc domain.

In an additional aspect, the polypeptide composition has variant Fc domains that are variant human IgG1 Fc domains comprising the amino acid substitutions M428L/N434S.

In a further aspect, the polypeptide composition has variant Fc domains that are variant human IgG1 Fc domains comprising the amino acid substitutions E233P/L234V/L235A/G236del/S267K.

In an additional aspect, the polypeptide composition has first and second variant Fc domains that comprise a set of heterodimerization variants selected from the group consisting of those depicted in FIGS. 2A-2B. In some embodiments, the set of heterodimerization variants is selected from the group consisting of L368D/K370S:S364K/E357Q; L368D/K370S:S364K; L368E/K370S:S364K; T41I E/K360E/Q362E:D401K; and T366S/L368A/Y407V:T366W.

In a further aspect, the polypeptide composition is selected from the group consisting of XENP27564 (SEQ ID NOs:297 and 298), XENP27563 (SEQ ID NOs:295 and 296), XENP26105 (SEQ ID NOs:245 and 246), and XENP26109 (SEQ ID NOs:249 and 250).

Also provided are nucleic acid compositions comprising: a) a first nucleic acid encoding said first protein monomer of any of the claims; and b) a second nucleic acid encoding said second protein monomer of any of the claims, respectively. Additionally, provided are expression vector compositions comprising a) a first expression vector comprising said first nucleic acid; and b) a second expression vector comprising said second nucleic acid, and host cells comprising the nucleic acid compositions and/or expression vector compositions. Further provided are methods of making a polypeptide composition comprising culturing the host cell of the invention under conditions wherein said composition is produced and recovering said composition.

In a further aspect, the IL-2-Fc fusion dimeric protein is selected from XENP24635; XENP24636; XENP24637; XENP24638; XENP24639; XENP24640; XENP24641; XENP24642; XENP24643; XENP24725; XENP24728; XENP24729; XENP24730; XENP24731; XENP24732; XENP25717; XENP25720; XENP25725; XENP25727; XENP25910; XENP25911; XENP25912; XENP26086; XENP26088; XENP26089; XENP26092; XENP26093; XENP26096; XENP26104; XENP26105; XENP26108; XENP26109; XENP26835; XENP26839; XENP26840; XENP26841; XENP26986; XENP26987; XENP26989; XENP26990, XENP26991, XENP25906, XENP25907; XENP25908; XENP25909; XENP26992; XENP26993; XENP26994; XENP26995; XENP26996; XENP27001; XENP27002; XENP27003; XENP27004; XENP27005; XENP27006 and XENP27007.

Additional aspects provide methods of activating CD25+ cells comprising contacting said cells with an IL-2-Fc fusion dimeric protein of the invention, and methods of treating autoimmune disease comprising administering to a patient in need thereof a protein composition herein.

### V. BRIEF DESCRIPTION OF THE DRAWINGS

FIGS. 1A-1B depicts the amino acid sequences (and GenBank accession numbers) of human IL-2 and its receptors: IL-2Rα (also known as CD25), IL-2Rβ (also known as CD122), and common gamma chain (also known as IL-2Ry or CD132).

FIGS. 2A-2E depicts useful pairs of depict useful pairs of Fc heterodimerization variant sets (including skew and pI variants). In FIGS. 2A-2E, there are variants for which there are no corresponding “monomer 2” variants; these are pI variants which can be used alone on either monomer.

FIG. 3 depict a list of isosteric variant antibody constant regions and their respective substitutions. pI (−) indicates lower pI variants, while pI_(+) indicates higher pI variants. These can be optionally and independently combined with other heterodimerization variants of the inventions (and other variant types as well, as outlined herein.)

FIG. 4 depict useful ablation variants that ablate FcγR binding (sometimes referred to as “knock outs” or “KO” variants). Generally, ablation variants are found on both monomers, although in some cases they may be on only one monomer.

FIGS. 5A-5B shows particularly useful embodiments of “non-cytokine” components of the invention.

FIGS. 6A-6D shows the sequences of several useful IL-2-Fc fusion format backbones based on human IgG, without the cytokine sequences. Backbone 1 is based on human IgG1 (356E/358M allotype), and includes C220S on both chain, the S364K/E357Q: L368D/K370S skew variants, the Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes C220S on both chain, the S364K/E357Q: L368D/K370S skew variants, the Q295E/N384D/Q418E/N421D pI variants on the chain with L368D/K370S skew variants and the E233P/L234V/L235A/G236del/S267K ablation variants on both chains. Backbone 3 is based on human IgG1 (356E/358M allotype), and includes C220S on both chain, the S364K/E357Q: L368E/K370S skew variants, the Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes C220S on both chain, the D401K: K360E/Q362E/T411E skew variants, the Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes C220S on both chain, the S364K/E357Q: L368D/K370S skew variants, the Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes C220S on both chain, the S364K/E357Q: L368D/K370S skew variants, Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Alternative formats for backbones 6 and 7 can exclude the ablation variants E233P/L234V/L235A/G236del/S267K in both chains. Backbone 8 is based on human IgG4, and includes the S364K/E357Q: L368D/K370S skew variants, the Q295E/N384D/Q418E/N421D pI variants on the chain with L368D/K370S skew variants, as well as a S228P (EU numbering, this is S241P in Kabat) variant on both chains that ablates Fab arm exchange as is known in the art. Backbone 9 is based on human IgG2, and includes the S364K/E357Q: L368D/K370S skew variants, the Q295E/N384D/Q418E/N421D pI variants on the chain with L368D/K370S skew variants. Backbone 10 is based on human IgG2, and includes the S364K/E357Q: L368D/K370S skew variants, the Q295E/N384D/Q418E/N421D pI variants on the chain with L368D/K370S skew variants as well as a S267K variant on both chains. Backbone 11 is identical to backbone 1, except it includes M428L/N434S Xtend mutations. Backbone 12 is based on human IgG1 (356E/358M allotype), and includes C220S on both identical chain, the the E233P/L234V/L235A/G236del/S267K ablation variants on both identical chains. Backbone 13 is based on human IgG1 (356E/358M allotype), and includes C220S on both chain, the S364K/E357Q: L368D/K370S skew variants, the P217R/P229R/N276K pI variants on the chain with S364K/E357Q skew variants and the E233P/L234V/L235A/G236del/S267K ablation variants on both chains.

As will be appreciated by those in the art and outlined below, any IL-2 variants can be incorporated into these backbones in any combination. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure. In particular, FcRn variants can also be included, such as M428L/N434S.

FIG. 7 depicts a number of exemplary variable length linkers. In some embodiments, these linkers find use linking the C-terminus of IL-2 to the N-terminus of the Fc region (which in some cases, includes the hinge domain).

FIGS. 8A and 8B depicts A) a structural model of the IL-2 complexed with the high-affinity IL-2 receptor (IL-2Rαβγ), and B) the location of three IL-2 residues which contact IL-2Rα at which substitutions were predicted to attenuate the pH dependent binding of IL-2 to IL-2Ra.

FIGS. 9A-9H depicts the amino acid sequences for illustrative IL-2 variants engineered to attenuate pH dependent binding to IL-2Ra. It is important to note that these variants were generated using polyhistidine (His×8 or HHHHHHHH) C-terminal tags which have been removed from the sequences depicted here.

FIGS. 10A-10C depicts the association rates (kₐ), dissociation rates (kₐ), and dissociation constants (K_{D}) of IL-2 variants to IL-2Rα at pH 6.0, as well as fold improvement in kd and KD over XENP14135 (wild-type IL-2 with C125S mutation). NB indicates no measurable binding.

FIGS. 11A-11C depicts the association rates (kₐ), dissociation rates (kₐ), and dissociation constants (K_{D}) of IL-2 variants to IL-2Rα at pH 7.4, as well as fold improvement in kd and KD over XENP14135 (wild-type IL-2 with C125S mutation). NB indicates no measurable binding.

FIG. 12 depicts the fold improvement in IL-2 off-rate (k_{d}) from IL-2Rα at pH 6.0 conveyed by various point mutations.

FIG. 13 depicts the fold improvement in IL-2 off-rate (k_{d}) from IL-2Rα at pH 7.4 conveyed by various point mutations.

FIG. 14 depicts the Biacore sensorgrams for A) XENP14135 (wild-type IL-2 with C125S mutation) and B) XENP14142 (variant IL-2 with R38I and C125S).

FIG. 15 depicts the amino acid sequences for an additional prior art IL-2 variant (mutant 2-4 with Q126T as described in WO 2009/061853, published May 14, 2009) without R38L (XENP14277) and with R38L (XENP14381) included to attenuate pH dependent binding to IL-2Ra. It is important to note that these variants were generated using polyhistidine (His×8 or HHHHHHHH) C-terminal tags which have been removed from the sequences depicted here.

FIG. 16 depicts the association rates (kₐ), dissociation rates (k_{d}), and dissociation constants (K_{D}) of IL-2 variants binding to IL-2Rα with and without R38L substitution at pH 7.4 and pH 6.0.

FIGS. 17A-17D depicts the amino acid sequences for illustrative IL-2 variants engineered for altered binding to IL-2Ra, IL-2Rβ, γc, or IL-2Rβγ. It is important to note that these variants were generated using polyhistidine (His×8 or HHHHHHHH) C-terminal tags which have been removed from the sequences depicted here.

FIGS. 18A-18B depicts the normalized BLI-response (relative to XENP14135) of illustrative IL-2 variants for the various IL-2 receptors as determined by Octet. The goal is to either increase the binding to IL-2Rα, or decrease the binding to IL-2Rβ and IL-2Rγ or to the interface of the IL-2Rβγ, or both.

FIGS. 19A-19D depicts several formats of the IL-2-Fc fusions of the present invention. Monovalent IL-2-Fc or “monovIL-2-Fc” (FIG. 19A) comprises IL-2 recombinantly fused to the N-terminus of a heterodimeric Fc-region, with the other side of the molecule being “Fc-only” or “empty-Fc”. Bivalent IL-2-Fc or “bivIL-2-Fc” (FIG. 19B) comprises IL-2 recombinantly fused to the N-terminus of both sides of a homodimeric Fc-region. Monovalent IL-2-Fc with linker or “monovIL-2-Fc (with linker)” (FIG. 19C) comprises IL-2 recombinantly fused to the N-terminus of a heterodimeric Fc-region via a domain linker, with the other side of the molecule being “Fc-only” or “empty-Fc”. Bivalent IL-2-Fc with linker or “bivIL-2-Fc (with linker)” (FIG. 19D) comprises IL-2 recombinantly fused to the N-terminus of both sides of a homodimeric Fc-region via a domain linker. Non-limiting examples of domain linkers suitable for use in monovIL-2-Fc (with linker) and bivIL-2-Fc (with linker) formats are depicted in FIG. 7.

FIGS. 20A-20E depicts the amino acid sequences for illustrative monovIL-2-Fc fusions of the invention. Slashes (/) indicate the border between IL-2 and Fc regions (in this case, the Fc region includes the hinge, as well as a C220S variant).

FIG. 21 depicts the affinity (K_{D}), association rate (kₐ), and dissociation rate (k_{d}) of illustrative IL-2-Fc fusions for IL-2Rα, IL-2Rβ, and IL-2Rβγ as determined by Octet.

FIGS. 22A-22F depicts induction of STAT5 phosphorylation on CD4⁺CD45RA⁻ T cells, CD4⁺CD45RA⁺ T cells, CD8⁺CD45RA⁺ T cells, CD8⁺CD45RA⁺ T cells, and Tregs by A) XENP24636, B) XENP24638, C) XENP24641, D) XENP24642, E) XENP24643, and F) XENP24731.

FIG. 23 depicts the amino acid sequences for illustrative bivIL-2-Fc fusions of the invention. Slashes (/) indicate the border between IL-2 and Fc regions, again with the Fc region including the hinge domain of IgG1 with a C220S variant.

FIGS. 24A and 24B depicts induction of STAT5 phosphorylation on CD4⁺CD45RA⁻ T cells, CD4⁺CD45RA⁺ T cells, CD8⁺CD45RA⁻ T cells, CD8⁺CD45RA⁺ T cells, and Tregs by A) XENP25906 and B) XENP25907.

FIGS. 25A-25B depicts the amino acid sequences for illustrative IL-2-Fc fusions with domain linkers. Slashes (/) indicate the border between IL-2, linkers, and Fc regions, again with the Fc region including the hinge domain of IgG1 with a C220S variant. Linkers are double underlined.

FIGS. 26A-26E depicts induction of STAT5 phosphorylation on CD4⁺CD45RA⁻ T cells, CD4⁺CD45RA⁺ T cells, CD8⁺CD45RA⁻ T cells, CD8⁺CD45RA⁺ T cells, and Tregs by A) XENP25908, B) XENP25909, C) XENP25910, D) XENP25911, and E) XENP25912.

FIGS. 27A-27E depicts amino acid sequences for additional IL-2-Fc fusions with IL-2 engineered for increased affinity to CD25 and decreased affinity for CD122. Slashes (/) indicate the border between IL-2 and Fc regions, again with the Fc region including the hinge domain of IgG1 with a C220S variant.

FIGS. 28A-28T depicts the phosphorylation of STAT5 on CD8⁺ and CD4⁺ T cells and Tregs as an indicator of activation by A) XENP24635, B) XENP24636, C) XENP24637, D) XENP24638, E) XENP24642, F) XENP25717, G) XENP25720, H) XENP25725, I) XENP25727, J) XENP26086, K) XENP26088, L) XENP26089, M) XENP26092, N) XENP26093, O) XENP26096, P) XENP26104, Q) XENP26105, R) XENP26108, S) XENP26109, and T) recombinant human IL-2.

FIGS. 29A and 29B depicts the phosphorylation of STAT5 on A) Tregs and B) CD4+(CD45RA−) T cells as an indicator of activation by illustrative IL-2-Fc fusions.

FIGS. 30A-30E depicts amino acid sequences for additional IL-2-Fc fusions. Slashes (/) indicate the border between IL-2, domain linkers (double underlined), and Fc regions, again with the Fc region including the hinge domain of IgG1 with a C220S variant.

FIGS. 31A-31E depicts some preferred embodiments.

FIG. 32 depicts some preferred embodiments of variants engineered with A) pH switch substitutions, B) Treg selectivity substitutions, and C) combination of pH switch and Treg selectivity substitutions.

FIG. 33 depicts the affinity (K_{D}), association rate (kₐ), and dissociation rate (k_{d}) of illustrative IL-2-Fc fusions for IL-2Rα, IL-2Rβ, and IL-2Rβγ as determined by Octet. N.B. indicates no binding.

FIG. 34 depicts the sequence for XENP27193, an Fc-IL-2(V91K/C125A) fusion engineered to increase the ratio of Tregs to non-regulatory T cells as described in WO 2014/153111. Slashes (/) indicate the border between IL-2, domain linker, and Fc region (again with the Fc region including the hinge domain of IgG1 with a C220S variant); and the linker is double-underlined.

FIGS. 35A to 35E depict induction of STAT5 phosphorylation on A) CD4⁺CD45RA⁻ T cells, B) CD8⁺CD45RA⁻ T cells, C) NK cells, D) γδ T cells, and E) Tregs by variant monovIL-2-Fc fusions XENP24638, XENP24642, XENP26105, XENP26109, XENP26835, XENP26839, XENP26991, and XENP25702. The data show that the variant monovIL-2-Fc fusions preferentially induced STAT5 phosphorylation on Tregs over CD4⁺ memory T cells (CD45RA⁻), CD8⁺ memory T cells (CD45RA⁻), NK cells, and γδ T cells in comparison to recombinant IL-2 and monovalent WT IL-2-Fc fusion (XENP24635) as well as prior art bivalent IL-2-Fc fusion described in WO 2014/153111 (XENP27193).

FIG. 36 depicts induction of STAT5 phosphorylation on CD4⁺CD45RA⁻ T cells and Tregs by IL-2 variant with N88R/C125S substitutions formatted as monovIL-2-Fc without linker (XENP24642) and bivIL-2-Fc with linker (XENP25908).

FIG. 37 depicts induction of STAT5 phosphorylation on CD4⁺CD45RA⁻ T cells and Tregs by IL-2 variant with T3A/D20N/C125S substitutions formatted as monovIL-2-Fc without linker (XENP25720), bivIL-2-Fc without linker (XENP26992), and bivIL-2-Fc with linker (XENP27002).

FIG. 38 depicts induction of STAT5 phosphorylation on CD4⁺CD45RA⁻ T cells and Tregs by IL-2 variant with T3A/D20N/T37R/C125S substitutions formatted as monovIL-2-Fc without linker (XENP26105), bivIL-2-Fc without linker (XENP26993), and bivIL-2-Fc with linker (XENP27003).

FIG. 39 depicts induction of STAT5 phosphorylation on CD4⁺CD45RA⁻ T cells and Tregs by IL-2 variant with T3A/D20N/N71K/C125S substitutions formatted as monovIL-2-Fc without linker (XENP26109), bivIL-2-Fc without linker (XENP26994), and bivIL-2-Fc with linker (XENP27004).

FIG. 40 depicts induction of STAT5 phosphorylation on CD4⁺CD45RA⁻ T cells and Tregs by IL-2 variant with T3A/D20N/R38I/C125S substitutions formatted as monovIL-2-Fc without linker (XENP26835), bivIL-2-Fc without linker (XENP26995), and bivIL-2-Fc with linker (XENP27005).

FIG. 41 depicts induction of STAT5 phosphorylation on CD4⁺CD45RA⁻ T cells and Tregs by IL-2 variant with T3A/D20N/T37R/R38I/C125S substitutions formatted as monovIL-2-Fc without linker (XENP26839), bivIL-2-Fc without linker (XENP26996), and bivIL-2-Fc with linker (XENP27006).

FIG. 42 depicts induction of STAT5 phosphorylation on CD4⁺CD45RA⁻ T cells and Tregs by IL-2 variant with T3A/D20N/R38I/N71K/C125S substitutions formatted as monovIL-2-Fc with linker (XENP26991), bivIL-2-Fc without linker (XENP27001), and bivIL-2-Fc with linker (XENP27007).

FIGS. 43A-43B depicts the amino acid sequences for illustrative bivalent IL-2-Fc fusions comprising Xtend (M428L/N434S) Fc of the invention (again including the hinge and a C220S variant). Slashes (/) indicate the border between IL-2 and Fc regions.

FIGS. 44A-44D depicts the amino acid sequences for illustrative monovalent IL-2-Fc fusions comprising Xtend (M428L/N434S) Fc of the invention. Slashes (/) indicate the border between IL-2 and Fc regions.

FIGS. 45A to 45E depicts the induction of STAT5 phosphorylation on various lymphocyte populations by A) XENP26105, B) XENP26109, C) XENP24635, D) XENP25908, and E) XENP27193.

FIG. 46 depicts immunophenotyping of rapamycin Treg cultures treated with XENP27564 or recombinant IL-2. The data show greater CD25 expression from treatment with XENP27564.

FIG. 47 Rapamycin Treg treated with XENP27564 or recombinant IL-2 are represented in a histogram to evaluate the relative expression of CD25. The data show that rapamycin Treg cultures treated with XENP27564 IL-2-Fc show greater CD25 expression.

FIG. 48 depicts various CD4⁺ compartments including Tregs following incubation with rapamycin and XENP27564 or recombinant IL-2. The data show that cultures expanded with XENP27564 show greater effector Treg population (CD45RA⁻FoxP3^{mid-high}) compared to cultures expanded with recombinant IL-2.

FIGS. 49A and 49B depicts suppression of A) CD8 responder T cell and B) CD4 responder T cell proliferation by rapamycin Treg cultures expanded with either XENP27564 or recombinant IL-2. The data suggests that Treg expanded by XENP27564 may have enhanced suppressive function.

FIGS. 50A-50B depicts expression of CD25 on Tregs in the suppression assay depicted in FIG. 47 as indicated by A) CD25 MFI on Tregs and B) percentage of CD25+ Tregs.

FIGS. 51A and 51B depict expression of CD127 on Tregs in the suppression assay depicted in FIG. 47 as indicated by A) CD127 MFI on Tregs and B) percentage of CD127+ Tregs.

FIGS. 52A to 52E depict proliferation (as determined by CFSE or Tag-it Violet dilution) of various lymphocyte populations following incubation or PBMCs and Tregs with A) XENP27563, B) XENP27564, C) XENP24635, D) recombinant IL-2, and E) recombinant IL-15. The data show that XENP27563 and XENP27564 show Treg selectivity.

FIGS. 53A to 53B depict proliferation of CD8+ T cells as indicated by A) proliferating cells (as determined by CFSE or Tag-it Violet dilution) and B) total cell counts following incubation or PBMCs and Tregs with XENP27563, XENP27564, XENP24635, recombinant IL-2, recombinant IL-15, and negative control anti-RSV mAb XENP15074. The data show that XENP27563 and XENP27564 are much less potent in induction of CD8+ T cell proliferation in comparison to recombinant IL-2 and IL-15 as well as IL-2-Fc fusion comprising WT IL-2 (with C125S mutation).

FIGS. 54A-54B depicts proliferation of CD4+ T cells as indicated by A) proliferating cells (as determined by CFSE or Tag-it Violet dilution) and B) total cell counts following incubation or PBMCs and Tregs with XENP27563, XENP27564, XENP24635, recombinant IL-2, recombinant IL-15, and negative control anti-RSV mAb XENP15074. The data show that XENP27563 and XENP27564 are much less potent in induction of CD4+ T cell proliferation in comparison to recombinant IL-2 and IL-15 as well as IL-2-Fc fusion comprising WT IL-2 (with C125S mutation).

FIG. 55 depicts proliferating CD8⁺ T cells (as indicated by percentage of cells expressing Ki67) following incubation of PBMC with indicated concentrations of indicated test articles and A) 5 ng/ml, B) 10 ng/ml, or C) 20 ng/ml plate-bound anti-CD3 (OKT3). The data show that XENP27563 and XENP27564 (IL-2-Fc fusions engineered for CD25-selectivity) have impaired function in induction of CD8⁺ T cell proliferation.

FIG. 56 depicts proliferating CD4⁺ T cells (as indicated by percentage of cells expressing Ki67) following incubation of PBMC with indicated concentrations of indicated test articles and A) 5 ng/ml, B) 10 ng/ml, or C) 20 ng/ml plate-bound anti-CD3 (OKT3). The data show that XENP27563 and XENP27564 (IL-2-Fc fusions engineered for CD25-selectivity) have impaired function in induction of CD4⁺ T cell proliferation.

FIG. 57 depicts proliferating CD8⁺CD45RA⁻ T cells (as indicated by percentage of cells expressing Ki67) following incubation of PBMC with indicated concentrations of indicated test articles and A) 5 ng/ml, B) 10 ng/ml, or C) 20 ng/ml plate-bound anti-CD3 (OKT3). The data show that XENP27563 and XENP27564 (IL-2-Fc fusions engineered for CD25-selectivity) have impaired function in induction of CD8⁺CD45RA⁻ T cell proliferation.

FIG. 58 depicts proliferating CD8⁺CD45RA⁺ T cells (as indicated by percentage of cells expressing Ki67) following incubation of PBMC with indicated concentrations of indicated test articles and A) 5 ng/ml, B) 10 ng/ml, or C) 20 ng/ml plate-bound anti-CD3 (OKT3). The data show that XENP27563 and XENP27564 (IL-2-Fc fusions engineered for CD25-selectivity) have impaired function in induction of CD8⁺CD45RA⁺ T cell proliferation.

FIG. 59 depicts proliferating CD4⁺CD45RA⁻ T cells (as indicated by percentage of cells expressing Ki67) following incubation of PBMC with indicated concentrations of indicated test articles and A) 5 ng/ml, B) 10 ng/ml, or C) 20 ng/ml plate-bound anti-CD3 (OKT3). The data show that XENP27563 and XENP27564 (IL-2-Fc fusions engineered for CD25-selectivity) have impaired function in induction of CD4⁺CD45RA⁻ T cell proliferation.

FIG. 60 depicts proliferating CD4⁺CD45RA⁺ T cells (as indicated by percentage of cells expressing Ki67) following incubation of PBMC with indicated concentrations of indicated test articles and A) 5 ng/ml, B) 10 ng/ml, or C) 20 ng/ml plate-bound anti-CD3 (OKT3). The data show that XENP27563 and XENP27564 (IL-2-Fc fusions engineered for CD25-selectivity) have impaired function in induction of CD4⁺CD45RA⁺ T cell proliferation.

FIG. 61 depicts proliferating NK cells (as indicated by percentage of cells expressing K167) following incubation of PBMC with indicated concentrations of indicated test articles and A) 5 ng/ml, B) 10 ng/ml, or C) 20 ng/ml plate-bound anti-CD3 (OKT3). The data show that XENP27563 and XENP27564 (IL-2-Fc fusions engineered for CD25-selectivity) have impaired function in induction of NK cell proliferation.

FIG. 62 depicts proliferating Tregs (as indicated by percentage of cells expressing Ki67) following incubation of PBMC with indicated concentrations of indicated test articles and A) 5 ng/ml, B) 10 ng/ml, or C) 20 ng/ml plate-bound anti-CD3 (OKT3). The data show that XENP27563 and XENP27564 (IL-2-Fc fusions engineered for CD25-selectivity) induce proliferation of Tregs.

FIG. 63 depicts proliferating CD8⁺ T cells, CD8⁺CD45RA⁻ T cells, CD8⁺CD45RA⁺ T cells, CD4+ T cells, CD4⁺CD45RA⁻ T cells, CD4⁺CD45RA⁺ T cells, NK cells, and Tregs (as indicated by percentage of cells expressing K167) following treatment with indicated concentrations of A) XENP27563, B) XENP27564, C) XENP24635, D) IL-2, and E) IL-15 and 5 ng/ml plate-bound anti-CD3 (OKT3).

FIG. 64 depicts proliferating CD8⁺ T cells, CD8⁺CD45RA⁻ T cells, CD8⁺CD45RA⁺ T cells, CD4+ T cells, CD4⁺CD45RA⁻ T cells, CD4⁺CD45RA⁺ T cells, NK cells, and Tregs (as indicated by percentage of cells expressing K167) following treatment with indicated concentrations of A) XENP27563, B) XENP27564, C) XENP24635, D) IL-2, and E) IL-15 and 10 ng/ml plate-bound anti-CD3 (OKT3).

FIG. 65 depicts proliferating CD8+ T cells, CD8⁺CD45RA⁻ T cells, CD8⁺CD45RA⁺ T cells, CD4⁺ T cells, CD4⁺CD45RA⁻ T cells, CD4⁺CD45RA⁺ T cells, NK cells, and Tregs (as indicated by percentage of cells expressing K167) following treatment with indicated concentrations of A) XENP27563, B) XENP27564, C) XENP24635, D) IL-2, and E) IL-15 and 20 ng/ml plate-bound anti-CD3 (OKT3).

FIGS. 66A-66D depicts expansion of A) CD4⁺CD45RA-T cells, B) CD8⁺CD45RA-T cells, C) CD8α CD16⁺ NK cells, and D) FoxP3⁺ Tregs in cynomolgus monkeys dosed with 3× dose XENP27563 and 3× dose XENP27564. The data show that both XENP27563 and XENP27564 selectively expanded Tregs, and that the two test articles promote similar pharmacology.

FIGS. 67A-67B depicts the change in serum concentration level over time in cynomolgus monkeys dosed with A) 3× dose XENP27563 and B) 3× dose XENP27564. The data indicates that the two test articles demonstrate similar pharmacokinetic profiles.

FIG. 68 depicts change in serum albumin concentration in cynomolgus monkeys dosed with 3× dose XENP27563 and 3× dose XENP27564. The data show that in one animal dosed with XENP27563, long-lasting albumin drops were detected after both the first and the second dosing; and in one animal dosed with XENP27564, a transient albumin drop was detected after the second dosing only with quick recovery to baseline levels.

FIGS. 69A-69C depicts blood pressure telemetry data for A) a first cynomolgus monkey dosed with XENP27563 on Day 0 and Day 15, B) a second cynomolgus monkey dosed with XENP27563 on Day 0 and Day 15, and C) a third cynomolgus monkey dosed with XENP27564 on Day 0 and 15. The data show a sharp blood pressure drop for the first monkey one day after the 2^{nd} dose, and a sharp blood pressure drop for the second monkey one day after the 1^{st} dose, while blood pressure remained stable in the third monkey throughout the duration of the study.

FIGS. 70A-70D depicts expansion of A) CD4⁺CD45RA⁻ T cells, B) CD8⁺CD45RA⁻ T cells, C) CD8α CD16⁺ NK cells, and D) Tregs over time in cynomolgus monkeys by 1×, 3×, and 10× dose of XENP27564.

FIG. 71 depicts the expansion of Tregs on Days 7 and 14 in cynomolgus dosed with 1× and 3× dose of XENP27564. The data show that 1× and 3× dose promote similar pharmacology in monkeys and suggest that a maximal effect is achievable at lower doses.

FIG. 72 depicts the change in serum albumin concentration (as an indicator of vascular leak) in cynomolgus monkey following dosing with 1×, 3×, and 10× dose of XENP27564. The data show that higher doses of XENP27564 increased toxicity.

FIG. 73 depicts the change in serum C-Reactive Protein concentration (as an indicator of vascular leak) in cynomolgus monkey following dosing with 1×, 3×, and 10× dose of XENP27564. The data show that higher doses of XENP27564 drastically increased toxicity.

FIGS. 74A-74D depicts change in A) sodium concentration, B) chloride concentration, C) eosinophil counts, and D) basophil counts in cynomolgus monkeys dosed with 1×, 3×, or 10× dose XENP27564. The data show that higher doses of XENP27564 increased toxicity, while lower doses are well tolerated in cynomolgus monkeys.

FIG. 75 depicts the change in serum concentration level over time in cynomolgus monkeys dosed with 1×, 3×, or 10× dose XENP27564. The data shows sustained pharmacokinetics up to several days in cynomolgus monkeys.

FIGS. 76A-76B depicts change in A) eosinophil counts and B) basophil counts in cynomolgus monkeys dosed with 3× dose XENP27563 or 3× dose XENP27563 on Days 0 and 15. The data show that repeat dosing of XENP27564 is well tolerated in cynomolgus monkeys.

FIGS. 77A-77C depicts induction of STAT5 phosphorylation on mouse CD4⁺CD44^{hi} cells, CD8⁺CD44^{hi} cells, and Tregs (in splenocytes from B6 mice) by A) XENP26105, B) XENP26109, and C) recombinant human IL-2. The data indicates that the engineered IL-2-Fc fusions are equally selective and potent for Tregs in mice making it suitable for using preclinical mouse models to investigate autoimmune diseases.

FIG. 78 depicts induction of STAT5 phosphorylation on human CD4⁺CD45RA⁻ T cells, CD4⁺CD45RA⁺ T cells, CD8⁺CD45RA⁻ T cells, CD8⁺CD45RA⁺ T cells, CD56+NK cells, γδ T cells, and Tregs by A) XENP27563 and B) XENP27564.

FIG. 79 depicts induction of STAT5 phosphorylation on cynomolgus CD4⁺CD45RA⁻ T cells, CD4⁺CD45RA⁺ T cells, CD8⁺CD45RA⁻ T cells, CD8⁺CD45RA⁺ T cells, CD16+NK cells, CD56+NK cells, γδ T cells, and Tregs by A) XENP27563 and B) XENP27564. The data indicates that the engineered IL-2-Fc fusions are equally selective and potent for Tregs in cynomolgus monkeys making it suitable for using preclinical mouse models.

### VI. DETAILED DESCRIPTION OF THE INVENTION

### A. Introduction

The present invention is directed to compositions and methods directed to engineered IL-2 Fc fusions for the treatment of autoimmune diseases. Autoimmune diseases can be treated using mechanisms that preferentially activate regulatory T cells, generally referred to as “Treg cells” or “Tregs”. Tregs are a subpopulation of immune T cells which are immunosuppressive and modulate the immune system by maintaining tolerance to self-antigens to prevent autoimmune disease. Tregs generally downregulate proliferation of effector T cells. Tregs express biomarkers including CD4, FOXP3 and CD25 (CD25 is also known as the IL-2Rα protein).

Tregs can be regulated by IL-2, which is essential for Treg function and survival. Because of the potential of IL-2 to promote or reduce both T cells and Tregs with limited selectivity, there is a strong need in the field to create more selective Treg modulators. Additionally, as a potential drug, IL-2 suffers from a very fast clearance, with half-life measured in minutes, which hinders favorable dosing. The present invention solves both these problems by providing novel IL-2-Fc fusion proteins.

Thus the invention provides IL-2 proteins that are engineered in two different ways. The first is that the IL-2 variants of the invention preferentially activate CD25+ cells such as Tregs over other T cells that are CD25−, to provide increased Treg selectivity over other T cells, and thus leading to compositions to suppress immune function and thus allow the treatment of autoimmune diseases. This is generally done by either increasing the binding to IL-2Rα, or decrease the binding to IL-2Rβ (and/or IL-2Rγ) or to the interface of the IL-2Rβγ, or both.

In addition to the selectivity engineering described above, the invention also provides IL-2 proteins that have increased serum half-life, which is done using Fc fusions. In this case, the addition of an Fc domain will increase the half-life of the IL-2 molecule, as is generally known in the art. However, the invention provides two additional methods for increasing the serum half-life.

The first involves the FcRn receptor. In IgG, a site on Fc between the Cy2 and Cy3 domains mediates interaction with the neonatal receptor FcRn. Binding to FcRn recycles endocytosed antibody from the endosome back to the bloodstream (Raghavan et al., 1996, Annu Rev Cell Dev Biol 12:181-220; Ghetie et al., 2000, Annu Rev Immunol 18:739-766, both entirely incorporated by reference). This process, coupled with preclusion of kidney filtration due to the large size of the full-length molecule, results in favorable antibody serum half-lives ranging from one to three weeks. In order to increase the retention of the Fc proteins in vivo, the increase in binding affinity must be at around pH 6 while maintaining lower affinity at around pH 7.4. Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood.

Accordingly, increased serum half life can utilize Fc variants that increase binding to FcRn and in many cases result in increased half life.

An additional method of increasing the serum half-life of the IL-2 Fc fusion molecule is based on pH engineering for recycling from the endosomal sorting pathway. As is known in the art, endocytosis of cytokines such as IL-2 into the endosome results in endocytotic sorting, where the cytokine either is degraded or is recycled back into the bloodstream (see Fallon et al., JBC 275(10):6790, 2000, hereby incorporated by reference in its entirety). Subsequent to internalization into the endosome, IL-2, IL-2Rβ, and γc are degraded, while IL-2Rα is constitutively recycled to the cell-surface. As the pH of blood is roughly 7.2 to 7.4, and the pH of the endosome is around pH 6, by engineering the IL-2 for increased binding to the IL-2Rα ligand at pH 6, the IL-2/IL-2Rα is recycled rather than degraded, resulting in an increased serum half-life.

Additionally, the potency of the IL-2 Fc fusion molecules of the invention can also depend on other factors. For example, the present invention provides for bivalent IL-2 constructs, such as depicted in FIG. 19B, where homodimers of variant IL-2-Fc fusions are made, thus providing bivalent binding to the receptors. Alternatively, the invention provides monovalent IL-2 constructs, such as depicted in FIG. 19A, where heterodimers are generated, one monomer of which is a variant IL-2-Fc fusion and the other is a “empty arm” Fc monomer. Additionally, the presence of additionally flexible linkers can increase potency in some cases, such as shown in FIG. 19C for the monovalent constructs and FIG. 19D for the bivalent constructs.

Accordingly, the present invention provides engineered IL-2 variants, as well as engineered IL-2 Fc fusion proteins, that show preferential activation of CD25+ cells such as Tregs over CD25− Tcells, and that exhibit increased serum half-life.

### B. Definitions

In order that the application may be more completely understood, several definitions are set forth below. Such definitions are meant to encompass grammatical equivalents.

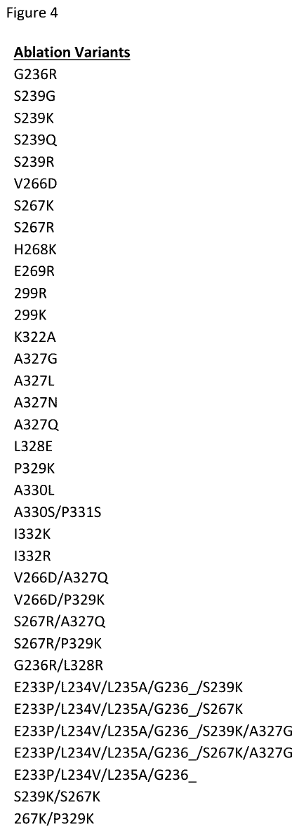

By “ablation” herein is meant a decrease or removal of activity. Thus for example, “ablating FcγR binding” means the Fc region amino acid variant has less than 50% starting binding as compared to an Fc region not containing the specific variant, with less than 70-80-90-95-98% loss of activity being preferred, and in general, with the activity being below the level of detectable binding in a Biacore assay. Of particular use in the ablation of FcγR binding are those shown in FIG. 4.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with the Fc region binding to FcγRIIIa; increased binding to FcγRIIIa leads to an increase in ADCC activity. As is discussed herein, many embodiments of the invention ablate ADCC activity entirely.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution S364K refers to a variant polypeptide, in this case an Fc variant, in which the serine at position 364 is replaced with lysine. The numbering is based on the numbering of the parent polypeptide. For example, R38W in the context of the IL-2 numbering. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233− or E233 #, E233( ) or E233del designates a deletion of glutamic acid at position 233. Additionally, EDA233− or EDA233 #designates a deletion of the sequence GluAspAla that begins at position 233.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. In general, the proteins of the invention utilize naturally occurring amino acids. In addition, variant polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Arginine 38 (also referred to as Arg38 or R38) is a residue at position 38 (numbering from the mature sequence) in the human IL-2 protein.

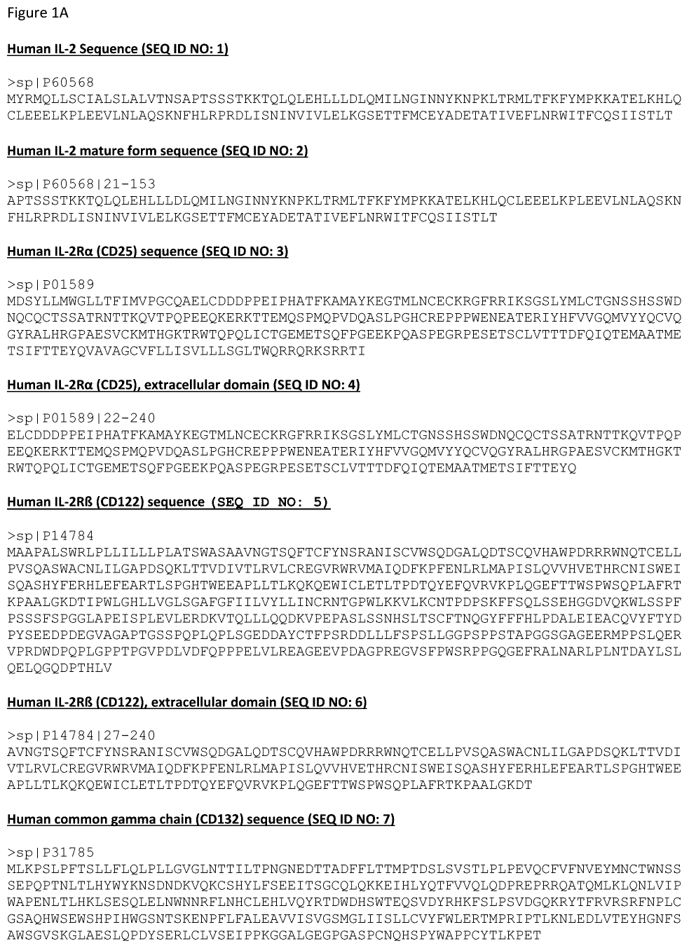

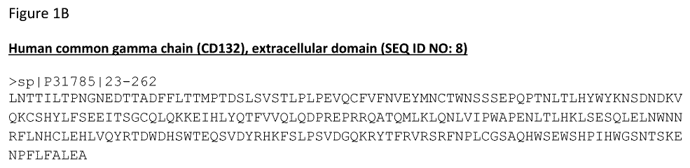

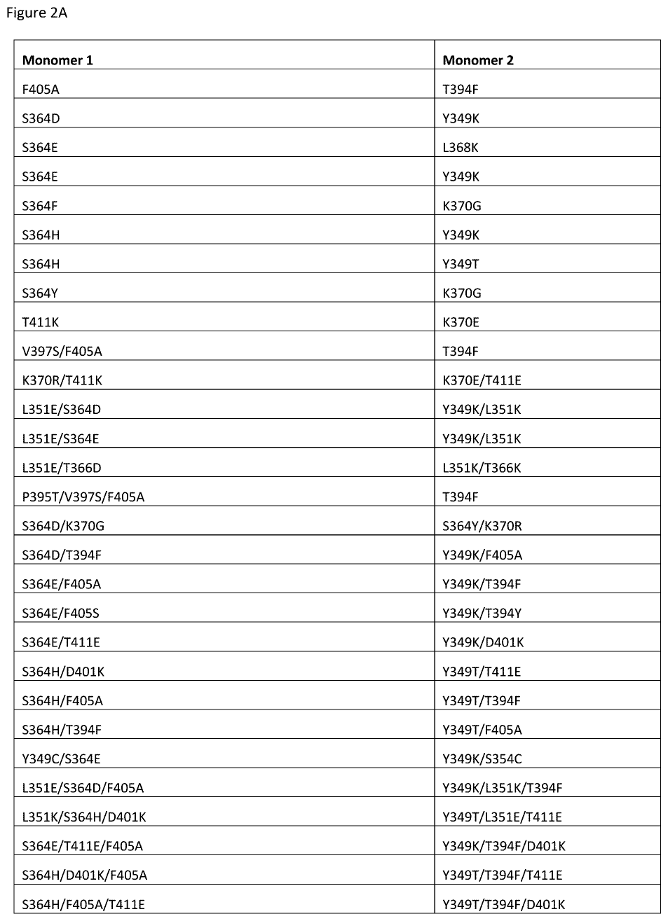

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from human IgG1, IgG2, IgG3 or IgG4. In the context of the IL-2 variants, the parent polypeptide is human IL-2, the mature sequence of which is shown in FIGS. 1A-1B. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it.

By “Fc” or “Fc region” or “Fc domain” as used herein is meant the polypeptide comprising the constant region of an IgG antibody excluding the first constant region immunoglobulin domain and in some cases, all or part of the hinge. For IgG, the Fc domain comprises immunoglobulin domains Cγ2 and Cγ3 (CH2 and CH3) and the hinge region between Cγ1 (CH1) and Cγ2 (CH2). In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat. Unless otherwise stated, the Fc domains of the invention include the hinge starting at position 216 (EU numbering) and end at the C-terminus of the CH3 domain at position 447; this is referred to as “hinge-CH2-CH3” of IgG. In some cases, such as in the Fc fusions herein, the hinge serves as a domain linker as discussed herein. In some embodiments, as is more fully described below, amino acid modifications are made to the Fc region, for example to alter binding to one or more FcγR receptors or to the FcRn receptor or to facilitate heterodimerization of Fc domains.

Accordingly, “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

By “interleukin-2” or “IL-2” herein is meant human IL-2 having the sequence shown in FIGS. 1A-1B.

By “IL-2 variant” or “variant IL-2” herein is meant a protein comprising an amino acid modification in the mature human IL-2 sequence shown in FIGS. 1A-1B. The IL-2 variants of the present invention are defined according to the amino acid modifications that compose them, as above, using the numbering of the mature human form.

By “Fc fusion protein” or “immunoadhesin” herein is meant a protein comprising an Fc region, generally linked (optionally through a linker moiety, as described herein, which can be the hinge region of an IgG, such as IgG1) to a different protein, such as IL-2. Thus an IL-2 Fc fusion protein is a protein comprising an IL-2 (in this case, variant IL-2) and Fc domains as outlined herein (again, generally Fc variants). These generally have the structure IL-2-hinge-CH2-CH3. As will be understood in the art, two Fc domains will self assemble to provide the dimeric Fc fusion proteins outlined herein.

By “position” as used herein is meant a location in the sequence of a protein. Positions may be numbered sequentially, or according to an established format, for example the EU index for antibody numbering.

By “non-naturally occurring modification” as used herein is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA 1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent IL-2” as used herein is meant an unmodified human IL-2 protein that is modified to generate a variant, and by “parent Fc” or “parent Fc domain” as used herein is meant an unmodified human IgG Fc domain that is modified to generate a variant Fc domain.

By “strandedness” in the context of the monomers of the heterodimeric Fc fusions of the invention herein is meant that, similar to the two strands of DNA that “match”, heterodimerization variants are incorporated into each monomer so as to preserve the ability to “match” to form heterodimers. For example, if some pI variants are engineered into monomer A (e.g. making the pI higher) then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “wild type or WT” herein is meant an amino acid sequence or a nucleotide sequence that is found in nature, including allelic variations. A WT protein has an amino acid sequence or a nucleotide sequence that has not been intentionally modified.

The proteins of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. “Recombinant” means the antibodies are generated using recombinant nucleic acid techniques in exogeneous host cells.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

By “linker” herein is meant a proteinaceous linker that is used to join two other protein domains (e.g. the variant IL-2 domain and the variant Fc domain). In some cases, the linker is a “domain linker”, used to link any two domains as outlined herein together. While any suitable linker can be used, many embodiments utilize a glycine-serine polymer, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers can be used. Additionally, the hinge domain of the human IgG1 protein can also be a domain linker.

By “regulatory T cells” or “Tregs” herein is meant T cells that are CD3+/CD4+/CD8-/CD25+/FOXP3+.

### VII. IL-2 FC FUSION PROTEINS OF THE INVENTION

The invention provides (IL-2)-(Fc domain) fusion proteins as shown herein and generally in FIG. 19A-19D. As will be appreciated by those in the art, the fusion proteins of the invention actually are two different polypeptides that self-assemble due to the presence of the Fe domains into either homodimeric proteins (FIG. 19B) or heterodimeric proteins (FIG. 19A). The proteins of the invention generally have three different domains: a Fc domain, one or more domain linkers, and an IL-2 domain.

### A. IL-2 Domains of the Invention

The IL-2 Fc fusion proteins of the invention include IL-2 domains, which are variant human IL-2 domains. As discussed herein, these domains are engineered to include specificity variants that lead to increased activation of T cells that are CD25+, such as Tregs, as compared to CD25− T cell populations, and optionally also include amino acid substitutions designed to increase the binding of the IL-2 to IL-2Rα at pH 6, such that the IL-2 variant Fc fusion proteins are recycled through the endocytotic pathway rather than degraded.

### 1. Expression Variants

As a preliminary matter, the IL-2 variants of the invention also include the C125S variant, which has been shown previously to increase expression of human I1-2. Thus, unless otherwise noted, all of the variants described herein include the C125S variant; in some cases, a C125A variant can also be used.

In addition, in some cases the IL-2 variants of the invention include a T3A variant, which removes an O-glycosylation site, to reduce complexity.

Additionally, the IL-2 variants of the invention include additional mutations.

### 2. Specificity Variants

Accordingly, the invention provides IL-2 variant proteins with increased specificity to CD25+ T cells, including Tregs. This is generally done by either increasing the binding to IL-2Rα, or decrease the binding to IL-2Rβ and IL-2Ry or to the interface of the IL-2Rβγ, or both.

In one embodiment, the variant IL-2 comprises a D20N amino acid substitution, in addition to the C125S expression variant, and thus has the amino acid set D20N/C125S. It should be noted that the D20N variant has previously been reported as resulting in a loss of binding for both the high affinity receptor (IL-2Rα 3) and the intermediate affinity receptor (IL-2Rβ); see Collins et al., PNAS USA 85:7709-7713 (1988), suggesting that “differential binding or activation between IL-2Rβγ or IL-2Rαβγ is not achievable by substitution of Asp at position 20”, see U.S. Pat. No. 6,955,807, Description of Related Art.

In one embodiment, the variant IL-2 comprises a T3A amino acid variant in addition to the D20N amino acid substitution and the C125S expression variant, and thus has the amino acid set T3A/D20N/C125S.

In one embodiment, the variant IL-2 comprises a T37R amino acid variant in addition to the T3A amino acid variant, the D20N amino acid substitution and the C125S expression variant, and thus has the amino acid set T3A/D20N/T37R/C125S.

In one embodiment, the variant IL-2 comprises a N71K amino acid variant in addition to the T3A amino acid variant, the D20N amino acid substitution and the C125S expression variant, and thus has the amino acid set T3A/D20N/N71K/C125S.

In one embodiment, the variant IL-2 comprises the amino acid variants N29S/Y31H/K35R/T37A/K48E/N71R/N88D/189V in addition to the C125S expression variant, and thus has the amino acid set N29S/Y31H/K35R/T37A/K48E/N71R/N88D/189V/C125S.

a. Assays for Treg Specificity

As is known in the art, the activation of STAT5 proteins (STAT5a and STAT5b) by phosphorylation is one of the earlies signaling events mediated by IL-2. Thus, by looking at STAT5 phosphorylation on different T cell populations using the constructs of the invention, specificity can be evaluated.

Generally, as described in the Examples, assays for STAT5 phosphorylation are done using the methods as outlined in the Examples. As described in the Examples, generally 5 different cell types are tested, including CD4+/CD45RA⁺, CD4+/CD45RA−, CD8⁺CD45RA⁺, CD8⁺CD45RA- and Tregs (CD3+/CD4+/CD8−/CD25+/FOXP3+) to provide a sampling of other T cell types (CD45RA is expressed on memory T cells and not on naïve T cells, for example).

In general, increased activity is as compared to human wild-type IL-2.

### 3. pH Variants

Additionally, the invention provides IL-2 variant proteins with increased pH specificity, where binding at pH 6 (the pH of the endosome) is increased.

In this embodiment, the IL-2 variant can have one or more amino acid substitutions selected from R38A, R38D, R38E, R38F, R38G, R38H, R38I, R38K, R38L, R38M, R38N, R38P, R38Q, R38S, R38T, R38V, R38W, R38Y, T41A, T41D, T41E, T41F, T41G, T41H, T41I, T41K, T41L, T41M, T41N, T41P, T41Q, T41R, T41S, T41V, T41W, T41Y, F42A, F42D, F42E, F42G, F42H, F42I, F42K, F42L, F42M, F42N, F42P, F42Q, F42R, F42S, F42T, F42V, F42W and F42Y. In addition, these mutations can be combined with C125S.

In this embodiment, the IL-2 variant can have amino acid substitution(s) selected from R38Q/T41K, R38Q/41Q, R38E/T41K, R38Q/T41R, R38N/T41Q, R38Q/T41V, R38N/T41V, R38Q/T41M, R38Q/T41S, R38Q/T41L, R38N/T41M, T41I/F42Y, T41E/F42Y, T41D/F42Y, T41M/F42Y, T41Q/F42Y, T41E/F42H, T41E/F42L, T41E/F42P, R38Q/F42Y, R38N/T41R, R38N/T41K, R38V/T41R, R38P/T41R, T41E/F42K, T41D/F42K, T41M/F42K, T41Q/F42K, R38Q/F42K, T41I/F42K, R38N/F42K, T41H/F42K, R38Q/T41K/F42Y, R38Q/T41R/F42Y, R38Q/T41Q/F42Y, R38Q/T41V/F42Y, R38N/T41K/F42K, R38Q/T41H/F42K, R38Q/T41K/F42K, R38Q/T41Q/F42K, R38Q/T41V/F42K and R38Q/T41R/F42K. In addition, these mutations can be combined with C125S.

### 4. Useful IL-2 Variants

The invention provides a number of particularly useful IL-2 variants that have desired activities both alone as well as when fused to Fc domains, including both wild-type Fc domains as well as variant Fc domains as outlined herein. In addition, these IL-2 variants may be used in monovalent constructs (e.g. FIG. 19A) or in bivalent constructs (e.g. FIG. 19B).

In one embodiment, the variant IL-2 domain has the amino acid substitutions R38I/C125S and is used in a bivalent construct. In this embodiment, the variant IL-2 domain may be fused to a wild Fc domain from IgG2 or IgG4, for example. Alternatively, the variant IL-2 domain may be fused to a variant Fc domain, for example, one that contains ablation and FcRn variants.

In one embodiment, the variant IL-2 domain has the amino acid substitutions R38I/C125S and is used in a monovalent construct.

In one embodiment, the variant IL-2 domain has the amino acid substitutions R38L/C125S and is used in a monovalent construct.

In one embodiment, the variant IL-2 domain has the amino acid substitutions R38L/C125S and is used in a bivalent construct.

In one embodiment, the variant IL-2 domain has the amino acid substitutions D20N/C125S and is used in a bivalent construct.

In one embodiment, the variant IL-2 domain has the amino acid substitutions D20N/C125S and is used in a monovalent construct.

In one embodiment, the variant IL-2 domain has the amino acid substitutions T3A/D20N/C125S and is used in a bivalent construct.

In one embodiment, the variant IL-2 domain has the amino acid substitutions T3A/D20N/C125S and is used in a monovalent construct.

In one embodiment, the variant IL-2 domain has the amino acid substitutions N29S/Y31H/K35R/T37A/K48E/N71R/N88D/189V/C125S and is used in a bivalent construct.

In one embodiment, the variant IL-2 domain has the amino acid substitutions N29S/Y31H/K35R/T37A/K48E/N71R/N88D/189V/C125S and is used in a monovalent construct.

In one embodiment, the variant IL-2 domain has the amino acid substitutions T3A/D20N/T37R/C125S and is used in a bivalent construct.

In one embodiment, the variant IL-2 domain has the amino acid substitutions T3A/D20N/T37R/C125S and is used in a monovalent construct.

In one embodiment, the variant IL-2 domain has the amino acid substitutions T3A/D20N/N71K/C125S and is used in a bivalent construct.

In one embodiment, the variant IL-2 domain has the amino acid substitutions T3A/D20N/N71K/C125S and is used in a monovalent construct.

In one embodiment, the variant IL-2 domain has the amino acid substitutions T3A/D20N/T37R/R38I/C125S and is used in a bivalent construct.

In one embodiment, the variant IL-2 domain has the amino acid substitutions T3A/D20N/T37R/R38I/C125S and is used in a monovalent construct.

In one embodiment, the variant IL-2 domain has the amino acid substitutions T3A/D20N/R38I/N71K/C125S and is used in a bivalent construct.

In one embodiment, the variant IL-2 domain has the amino acid substitutions T3A/D20N/R38I/N71K/C125S and is used in a monovalent construct.

Particularly preferred proteins include XENP14142, XENP14144, XENP23833, XENP25720, XENP26086, XENP26105, XENP26987, XENP27003, XENP26109, XENP26994, XENP26841, XENP27004, XENP26839, XENP26996, XENP26990, XENP27006, XENP26840, XENP27001, XENP26991, XENP27007, XENP27563, XENP26105, XENP27564 and XENP26109.

### B. Fc Domains of the Invention

As discussed herein, the present invention provides Fc fusion proteins comprising two Fe domains with at least one of the Fc domains comprising a fused I1-2 variant, optionally including a domain linker. As shown in FIG. 19A-19D, the dimeric proteins of the invention can be configured to have one IL-2 variant, sometimes referred to herein as “monovalent IL-2” as shown in FIG. 19A, where one of the Fc domains is covalently attached to the IL-2 variant protein and the other is “empty” or is “Fc only”. This embodiment relies on heterodimeric Fc domains, as discussed below. Alternatively, “bivalent” IL-2 constructs such as shown in FIG. 19B are used, where each Fc domain is fused to an IL-2 variant; these embodiments utilize homodimeric Fc domains, as discussed above.

In both embodiments, whether a homo- or heterodimeric Fc fusion construct is used, the Fc domains generally include some specific amino acid variants for several functions.

### 1. Fc Variants for Additional Functionality

In addition to pI amino acid variants, there are a number of useful Fc amino acid modification that can be made for a variety of reasons, including, but not limited to, altering binding to one or more FcγR receptors, altered binding to FcRn receptors, etc.

Accordingly, the proteins of the invention can include amino acid modifications, including the heterodimerization variants outlined herein, which includes the pI variants and steric variants. Each set of variants can be independently and optionally included or excluded from any particular heterodimeric protein.

### (i) FcγR Variants

Accordingly, there are a number of useful Fc substitutions that can be made to alter binding to one or more of the FcγR receptors. Substitutions that result in increased binding as well as decreased binding can be useful. For example, it is known that increased binding to FcγRIIIa results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the present invention include those listed in U.S. Ser. No. 11/124,620 (particularly FIG. 41), Ser. Nos. 11/174,287, 11/396,495, 11/538,406, all of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

In addition, there are additional Fc substitutions that find use in increased binding to the FcRn receptor and increased serum half life, as specifically disclosed in U.S. Ser. No. 12/341,769, hereby incorporated by reference in its entirety, including, but not limited to, 434S, 434A, 428L, 308F, 259I, 428L/434S, 428L/434A, 259I/308F, 436I/428L, 436I or V/434S, 436V/428L and 259I/308F/428L.

### (ii) Ablation Variants

Similarly, another category of functional variants are “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g. FcγR1, FcγRIIa, FcγRIIb, FcγRIIIa, etc.) to avoid additional mechanisms of action. That is, for example, it is desirable to ablate FcγRIIIa binding to eliminate or significantly reduce ADCC activity such that one of the Fc domains comprises one or more Fcγ receptor ablation variants. These ablation variants are depicted in FIG. 4, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

### 2. Homodimeric Fc Domains

In some embodiments, the invention provides bivalent homodimeric proteins comprising homodimeric Fc domains, as generally depicted in FIG. 19B. In this embodiment, each monomer is identical, and generally comprises the variant-IL-2-linker-Fc domain, where the linker is generally the hinge from IgG1.

In this embodiment, the Fc domains can have ablation variants as generally shown in FIG. 4. Suitable ablation variants are shown in FIG. 4 that generally ablate binding to FcγRI, FcγRIIb and FcγRIIIa. Of particular use in this IgG1 embodiment is the ablation amino acid set of E233P/L234V/L235A/G236_/S267K (the “G236_” is a deletion as described herein).

In addition, there are additional Fc substitutions that find use in increased binding to the FcRn receptor and increased serum half life, as specifically disclosed in U.S. Ser. No. 12/341,769, hereby incorporated by reference in its entirety, including, but not limited to, 434S, 434A, 428L, 308F, 259I, 428L/434S, 428L/434A, 259I/308F, 436I/428L, 436I or V/434S, 436V/428L and 259I/308F/428L.

### 3. Heterodimeric Fc Domains

In addition to homodimeric, bivalent IL-2 fusion proteins, alternate embodiments utilize monovalent IL-2 fusion proteins, where one of the Fc domains is “empty” and the invention relies on heterodimerization variants to bring the two Fc domains together, as shown in FIG. 19B. These embodiments rely on the use of two different variant Fc sequences, that will self-assemble to form heterodimeric Fc domains and heterodimeric Fc fusion proteins.

The heterodimeric protein constructs are based on the self-assembling nature of the two Fc domains of the heavy chains of antibodies, e.g. two “monomers” that assemble into a “dimer”. Heterodimeric proteins are made by altering the amino acid sequence of each monomer as more fully discussed below. Thus, the present invention is generally directed to the creation of heterodimeric Fc fusion proteins that rely on amino acid variants in the constant regions that are different on each chain to promote heterodimeric formation and/or allow for ease of purification of heterodimers over the homodimers.

There are a number of mechanisms that can be used to generate the heterodimers of the present invention. In addition, as will be appreciated by those in the art, these mechanisms can be combined to ensure high heterodimerization. Thus, amino acid variants that lead to the production of heterodimers are referred to as “heterodimerization variants”. As discussed below, heterodimerization variants can include steric variants (e.g. the “knobs and holes” or “skew” variants described below and the “charge pairs” variants described below) as well as “pI variants”, which allows purification of homodimers away from heterodimers. As is generally described in WO2014/145806, hereby incorporated by reference in its entirety and specifically as below for the discussion of “heterodimerization variants”, useful mechanisms for heterodimerization include “knobs and holes” (“KIH”; sometimes herein as “skew” variants (see discussion in WO2014/145806), “electrostatic steering” or “charge pairs” as described in WO2014/145806, pI variants as described in WO2014/145806, and general additional Fc variants as outlined in WO2014/145806 and below.

In the present invention, there are several basic mechanisms that can lead to ease of purifying heterodimeric proteins; one relies on the use of pI variants, such that each monomer has a different pI, thus allowing the isoelectric purification of A-A, A-B and B-B dimeric proteins. Additionally, the heterodimeric proteins of the invention also allow separation on the basis of size. As is further outlined below, it is also possible to “skew” the formation of heterodimers over homodimers. Thus, a combination of steric heterodimerization variants and pI or charge pair variants find particular use in the invention.

In general, embodiments of particular use in the present invention rely on sets of variants that include skew variants, that encourage heterodimerization formation over homodimerization formation, coupled with pI variants, which increase the pI difference between the two monomers.

Additionally, as more fully outlined herein, pI variants can be either contained within the constant and/or Fc domains of a monomer, or charged linkers, such as domain linkers, can be used.

In the present invention that utilizes pI as a separation mechanism to allow the purification of heterodimeric proteins, amino acid variants can be introduced into one or both of the monomer polypeptides; that is, the pI of one of the monomers (referred to herein for simplicity as “monomer A”) can be engineered away from monomer B, or both monomer A and B change be changed, with the pI of monomer A increasing and the pI of monomer B decreasing. As discussed, the pI changes of either or both monomers can be done by removing or adding a charged residue (e.g. a neutral amino acid is replaced by a positively or negatively charged amino acid residue, e.g. glycine to glutamic acid), changing a charged residue from positive or negative to the opposite charge (e.g. aspartic acid to lysine) or changing a charged residue to a neutral residue (e.g. loss of a charge; lysine to serine). A number of these variants are shown in the Figures.

Accordingly, this embodiment of the present invention provides for creating a sufficient change in pI in at least one of the monomers such that heterodimers can be separated from homodimers. As will be appreciated by those in the art, and as discussed further below, this can be done by using a “wild type” heavy chain constant region and a variant region that has been engineered to either increase or decrease its pI (wt A−+B or wt A−−B), or by increasing one region and decreasing the other region (A+−B− or A−B+).

Thus, in general, a component of some embodiments of the present invention are amino acid variants in the Fc domains that are directed to altering the isoelectric point (pI) of at least one, if not both, of the monomers of a dimeric protein by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

a. Heterodimerization Variants

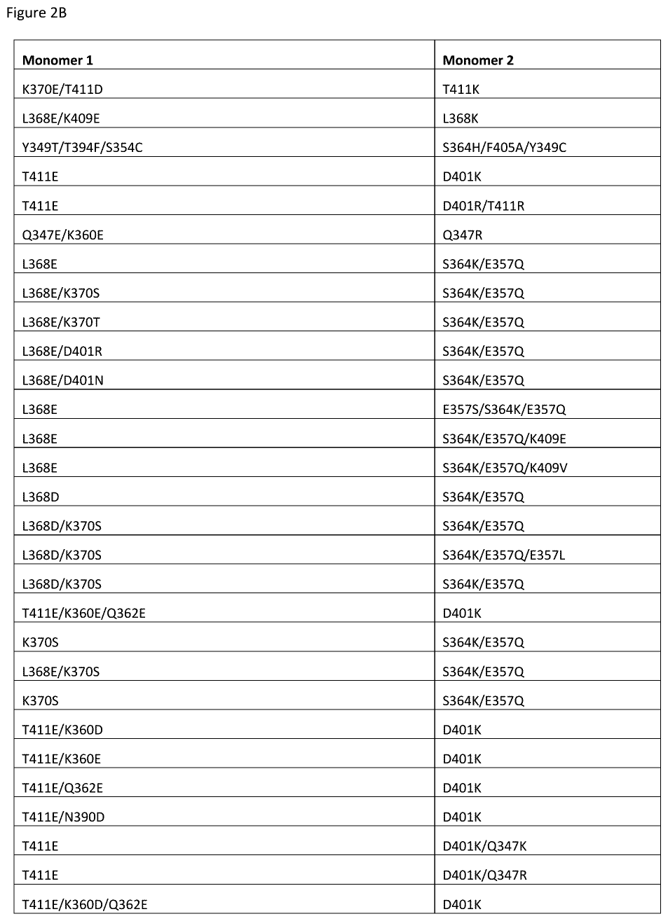

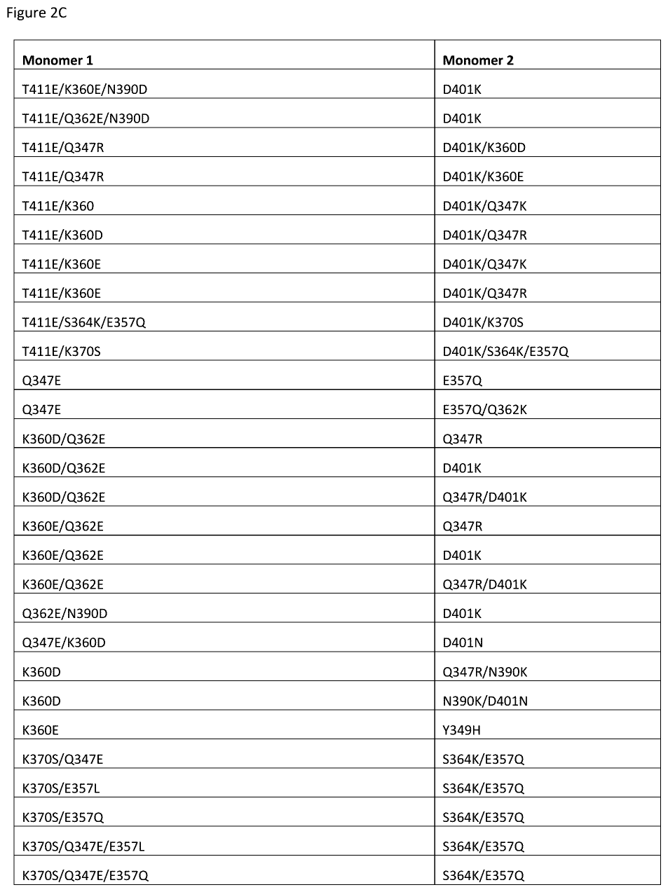

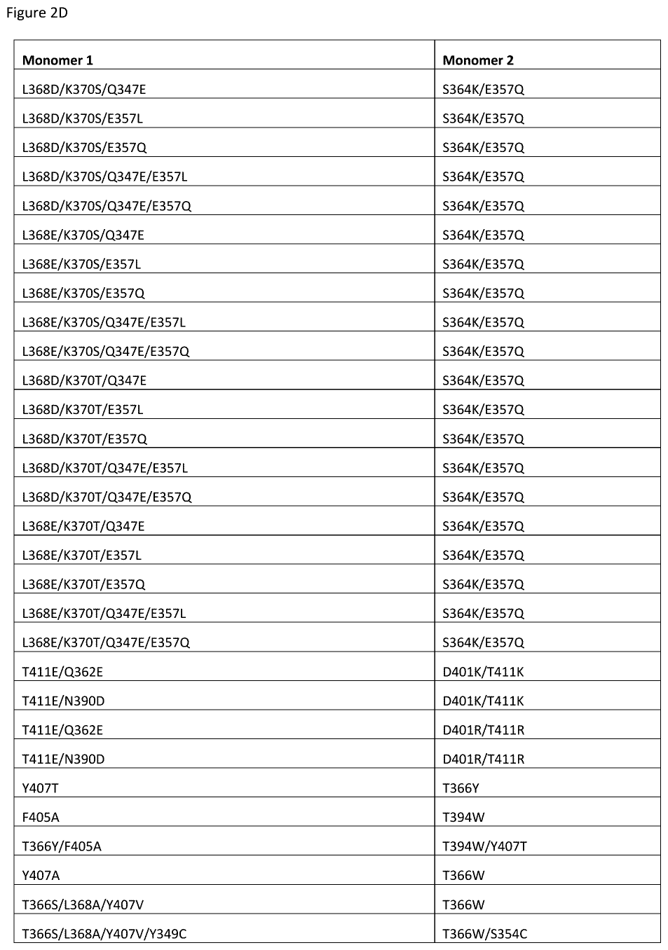

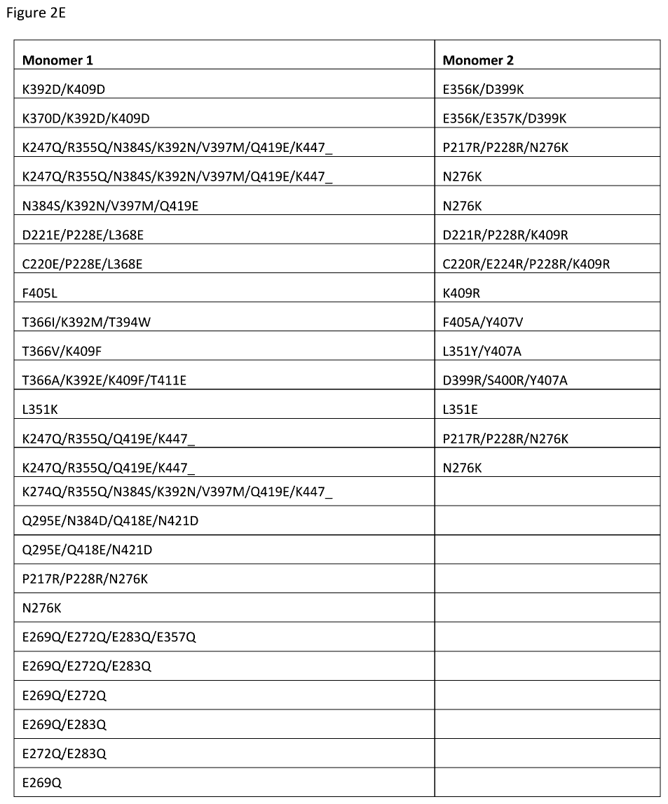

The present invention provides heterodimeric proteins, including heterodimeric antibodies in a variety of formats, which utilize heterodimeric variants to allow for heterodimeric formation and/or purification away from homodimers. A number of heterodimerization variants are shown in FIGS. 2A-2E.

There are a number of suitable pairs of sets of heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other; that is, these pairs of sets form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B).

b. Steric Variants

In some embodiments, the formation of heterodimers can be facilitated by the addition of steric variants. That is, by changing amino acids in each heavy chain, different heavy chains are more likely to associate to form the heterodimeric structure than to form homodimers with the same Fc amino acid sequences. Suitable steric variants are included in in the Figures.

One mechanism is generally referred to in the art as “knobs and holes”, referring to amino acid engineering that creates steric influences to favor heterodimeric formation and disfavor homodimeric formation can also optionally be used; this is sometimes referred to as “knobs and holes”, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety. The Figures identify a number of “monomer A-monomer B” pairs that rely on “knobs and holes”. In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g. these are “monomer corresponding sets) and C220E/P228E/368E paired with C220R/E224R/P228R/K409R.

Additional monomer A and monomer B variants that can be combined with other variants, optionally and independently in any amount, such as pI variants outlined herein or other steric variants that are shown in FIG. 37 of US 2012/0149876, the figure and legend and SEQ ID NOs of which are incorporated expressly by reference herein.

In some embodiments, the steric variants outlined herein can be optionally and independently incorporated with any pI variant (or other variants such as Fc variants, FcRn variants, etc.) into one or both monomers, and can be independently and optionally included or excluded from the proteins of the invention.

A list of suitable skew variants is found in FIGS. 2A-2E showing some pairs of particular utility in many embodiments. Of particular use in many embodiments are the pairs of sets including, but not limited to, S364K/E357Q: L368D/K370S; L368D/K370S:S364K; L368E/K370S:S364K; T411T/E360E/Q362E:D401K; L368D/K370S:S364K/E357L, K370S:S364K/E357Q and T366S/L368A/Y407V:T366W (optionally including a bridging disulfide, T366S/L368A/Y407V/Y349C:T366W/S354C). In terms of nomenclature, the pair “S364K/E357Q: L368D/K370S” means that one of the monomers has the double variant set S364K/E357Q and the other has the double variant set L368D/K370S; as above, the “strandedness” of these pairs depends on the starting pI.

c. pI (Isoelectric point) Variants for Heterodimers

In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

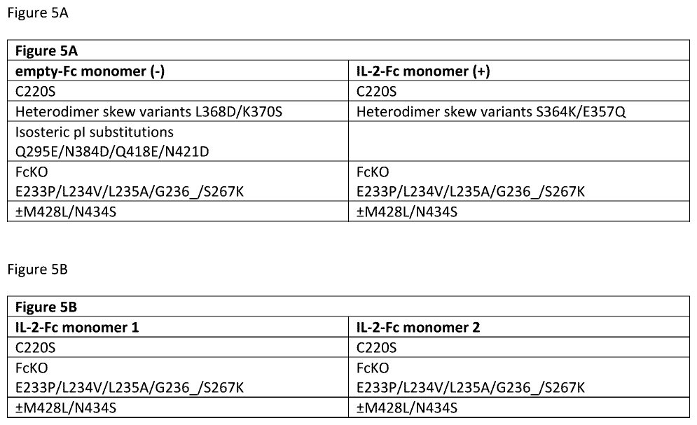

Preferred combinations of pI variants are shown in FIGS. 5A-5B. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative side) comprising 295E/384D/418E/421D variants (Q295E/N384D/Q418E/N421D when relative to human IgG1) and a second monomer (the positive side) comprising a positively charged scFv linker, including (GKPGS)₄.

d. Isotypic Variants

In addition, some embodiments of the invention rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g. by changing a higher pI amino acid to a lower pI amino acid), or to allow accommodations in structure for stability, etc. as is more further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

e. Calculating pI

The pI of each monomer can depend on the pI of the variant IL-2 domain and the Fc domain, as outlined herein. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

f. Combination of Heterodimeric and Fc Variants

As will be appreciated by those in the art, for use in heterodimer Fc domains, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew and pI, are also independently and optionally combined with Fc ablation variants, Fc variants, FcRn variants, as generally outlined herein.

In addition to ablation variants, the Fc domains generally also include a C220S variant (e.g. as the Fc domains of the invention include the hinge region starting at position 216, EU numbering), which is eliminated as there are no light chains included herein and this cysteine is used for disulfide formation with the light chain.

Additionally, the Fc domains of the fusion proteins of the invention optionally can comprise half life extension amino acid substitutions.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### VIII. USEFUL CONSTRUCTS OF THE INVENTION

As outlined herein, the invention provides a number of useful monovalent and bivalent constructs.

### A. Heterodimeric Monovalent Constructs

In some embodiments, the I1-2-Fc fusion proteins of the invention are heterodimeric monovalent constructs such as those depicted in FIGS. 19A and 19C. In this embodiment, the variant IL-2 domain is generally fused to a variant human IgG1 Fc domain using either the hinge as the domain linker (generally including a C220S variant) or using an additional linker attached to the hinge, with the other Fc domain (including the hinge) remaining “empty”.

In some embodiments, the variant IL-2 domain is attached to the “+” monomer side (see FIG. 5A), which includes a variant human IgG1 Fc domain (inclusive of the hinge with a C220S variant), S364K/E357Q “skew variants” and ablation variants E233P/L234V/L235A/G236del/S267K, and the “empty Fc side” is a variant human IgG1 Fc domain (inclusive of the hinge with a C220S variant), L368D/K370S “skew variants” and ablation variants E233P/L234V/L235A/G236del/S267K. In this embodiment, preferred constructs include a variant IL-2 domain with amino acid substitutions selected from the group consisting of T3A/D20N/T37R, T3A/D20N/T37R/C125S, T3A/D20N/T37R/C125A, T3A/D20N/N71K, T3A/D20N/N71K/C125S and T3A/D20N/N71K/C125A.

In some embodiments, the variant IL-2 domain is attached to the “+” monomer side (see FIG. 5A), which includes a variant human IgG1 Fc domain (inclusive of the hinge with a C220S variant), S364K/E357Q “skew variants” and ablation variants E233P/L234V/L235A/G236del/S267K, and the FcRn variants M428L/N434S, and the “empty Fc side” is a variant human IgG1 Fc domain (inclusive of the hinge with a C220S variant), L368D/K370S “skew variants” and ablation variants E233P/L234V/L235A/G236del/S267K and the FcRn variants M428L/N434S. In this embodiment, preferred constructs include a variant IL-2 domain with amino acid substitutions selected from the group consisting of T3A/D20N/T37R, T3A/D20N/T37R/C125S, T3A/D20N/T37R/C125A, T3A/D20N/N71K, T3A/D20N/N71K/C125S and T3A/D20N/N71K/C125A.

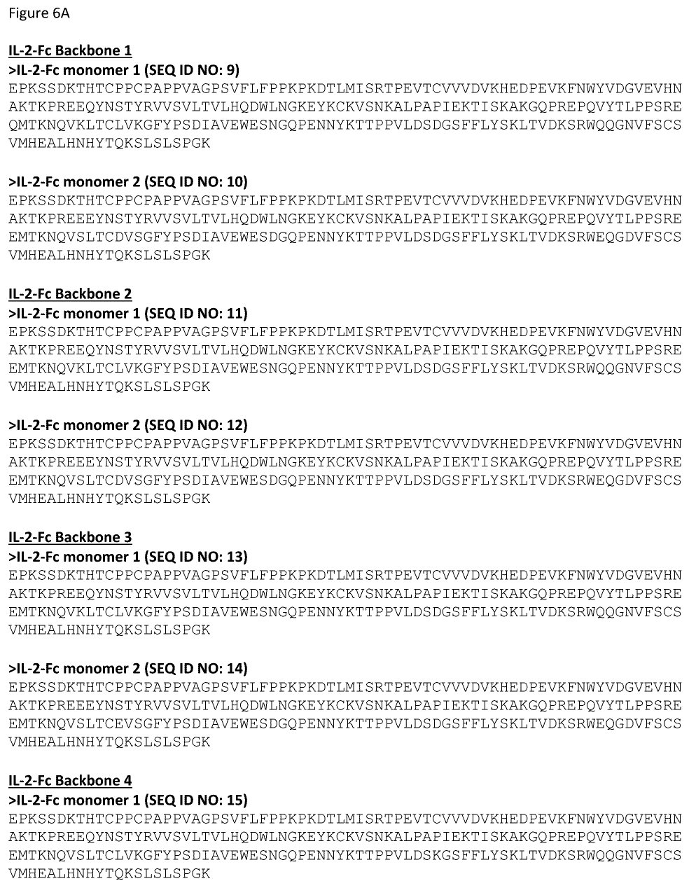

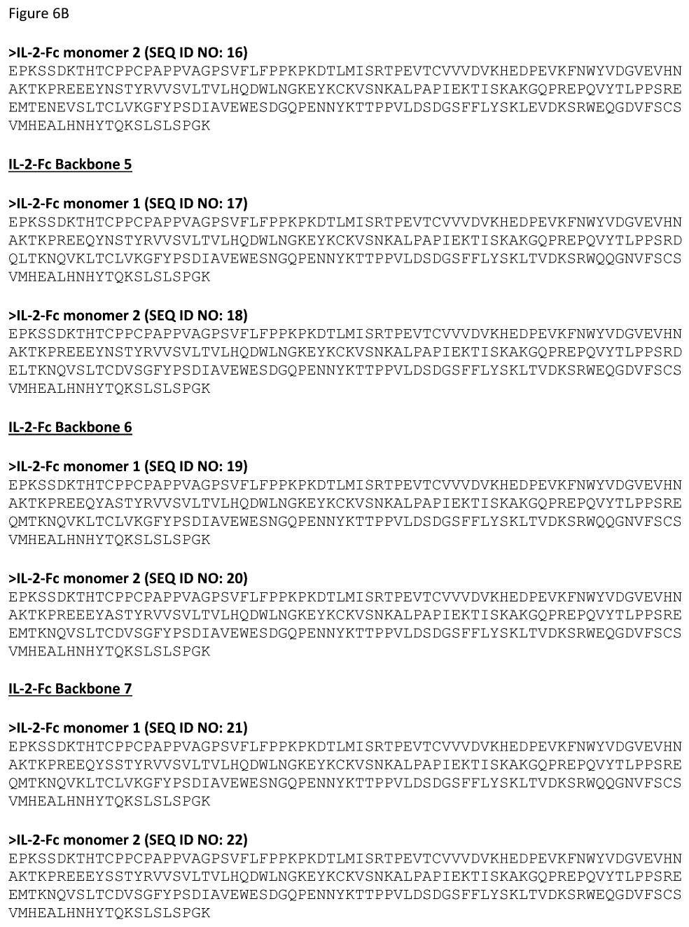

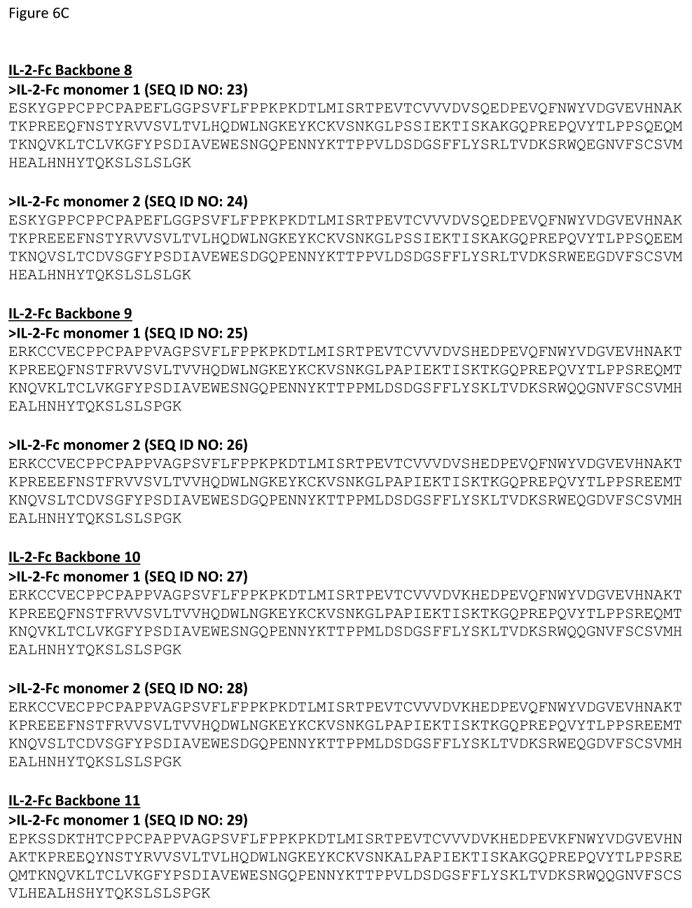

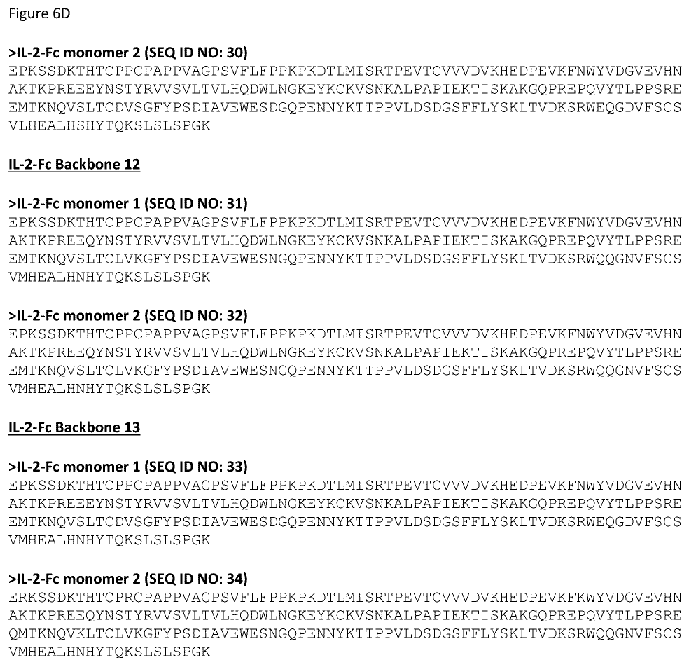

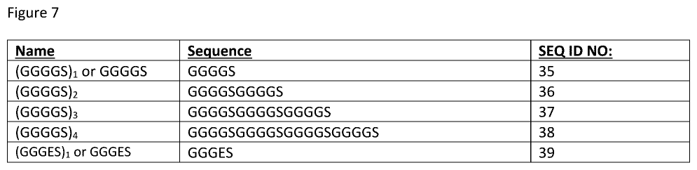

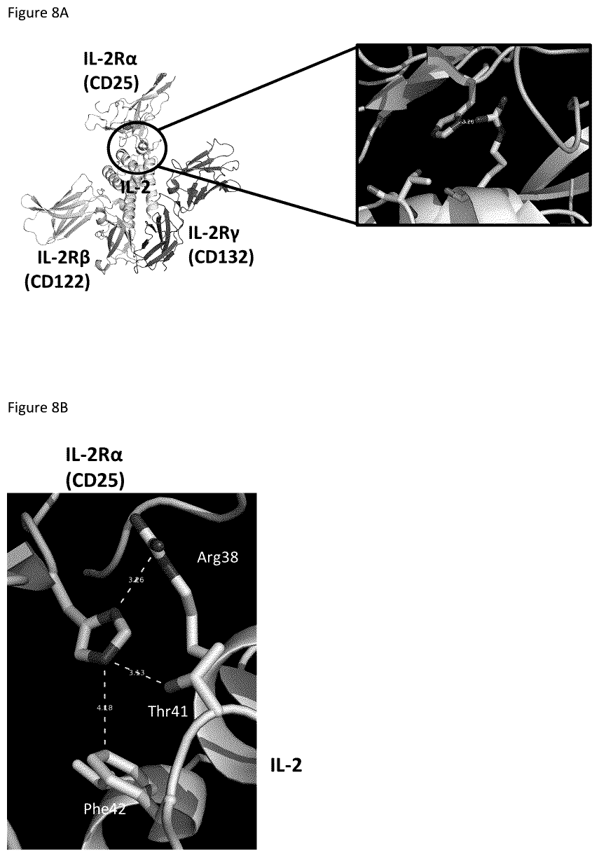

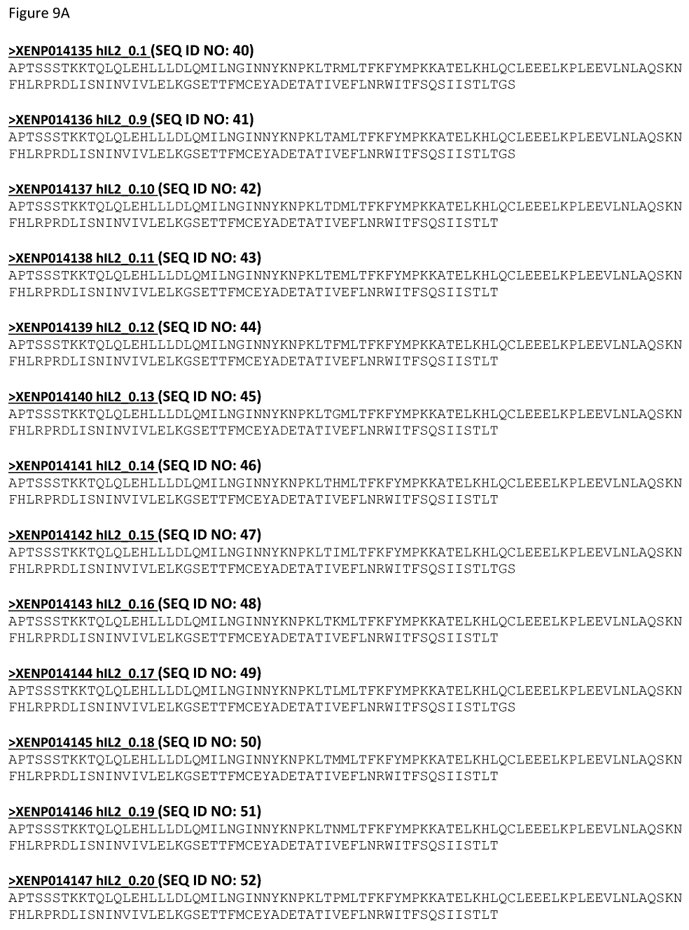

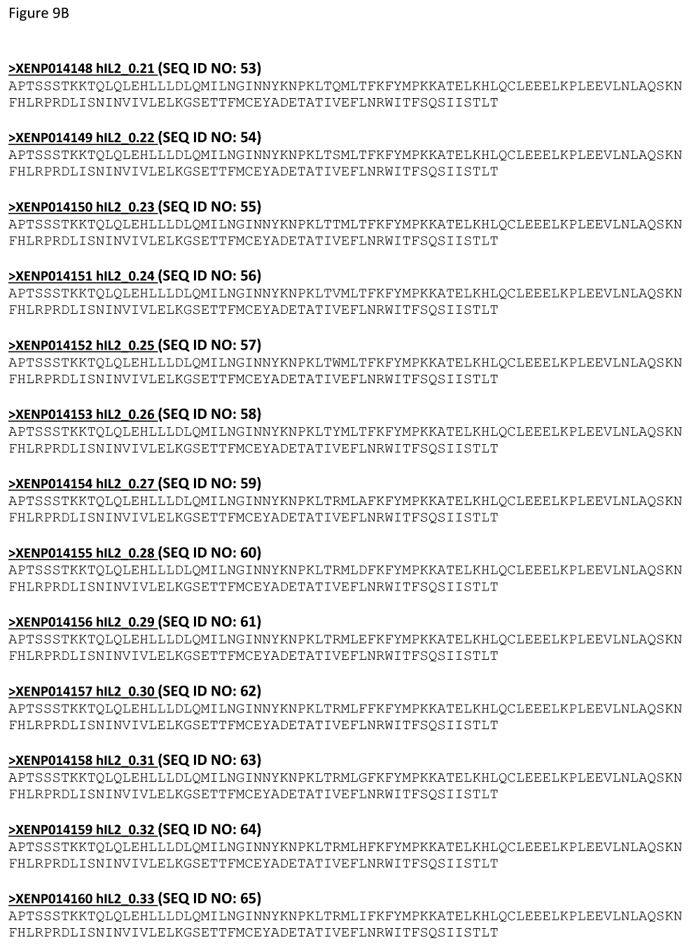

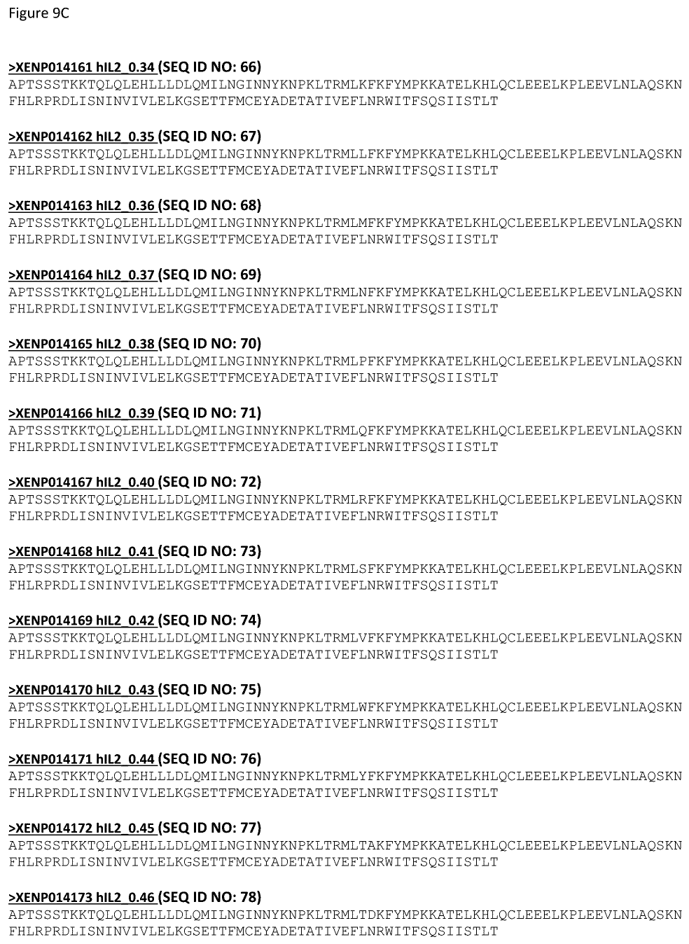

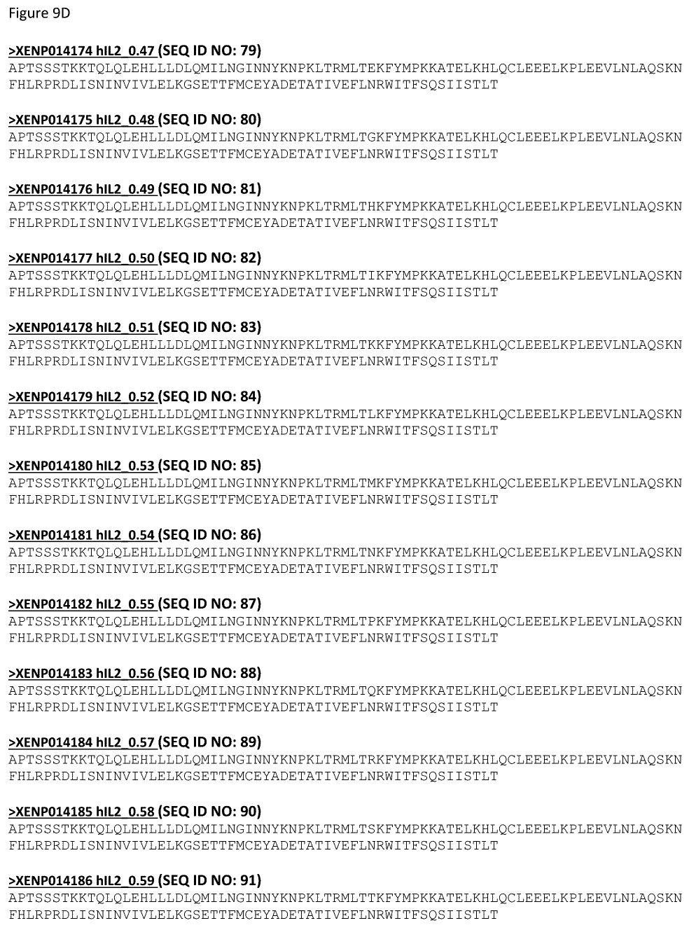

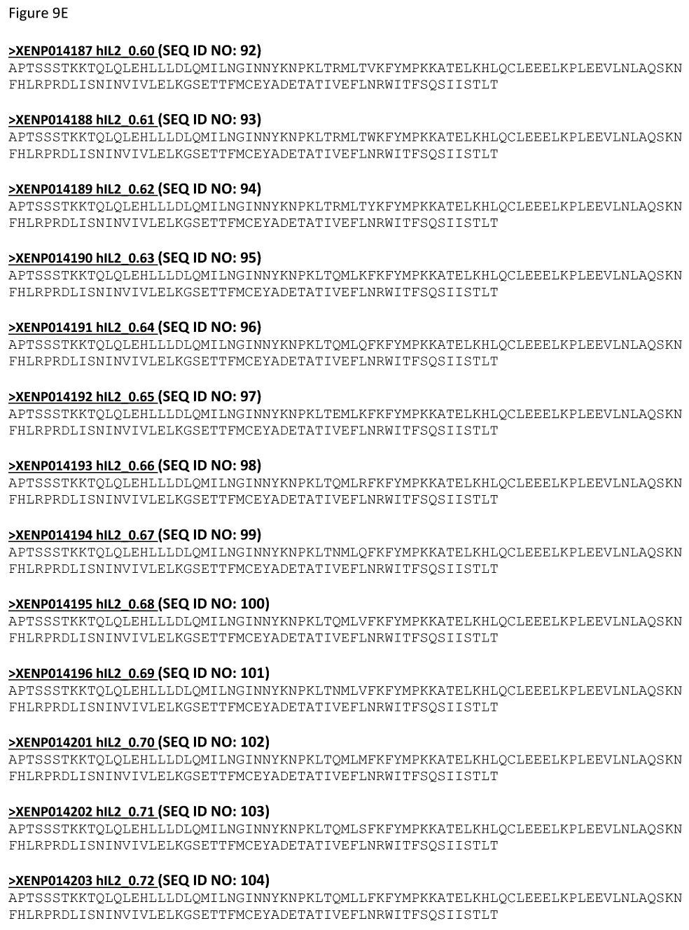

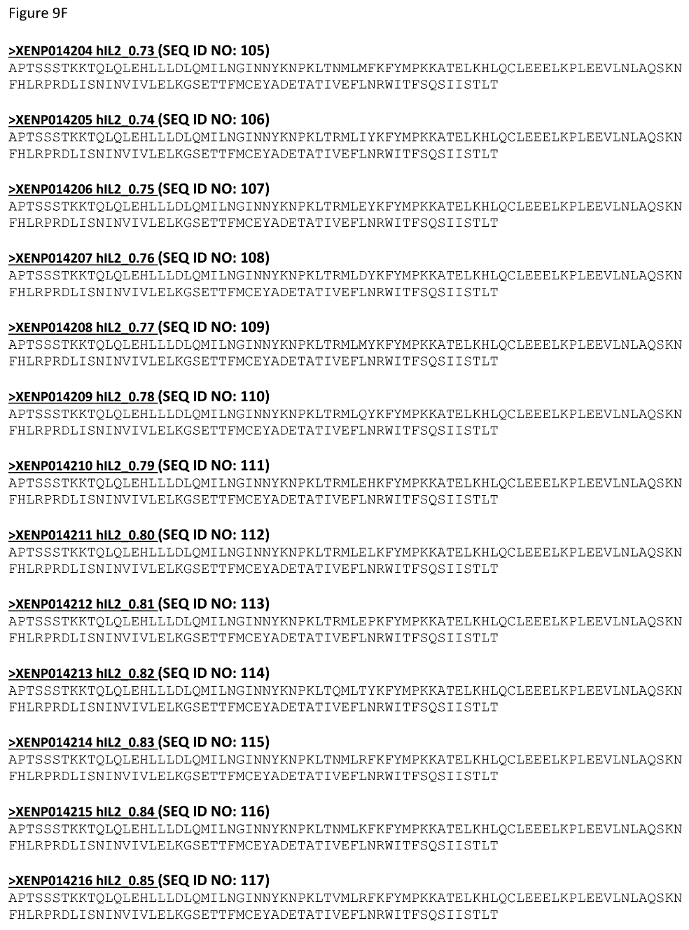

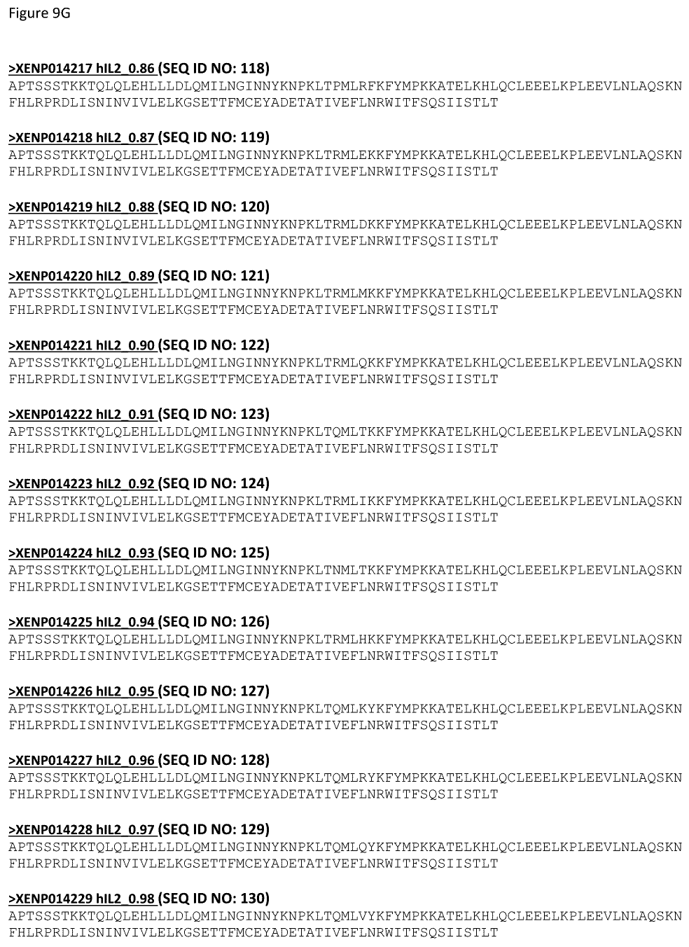

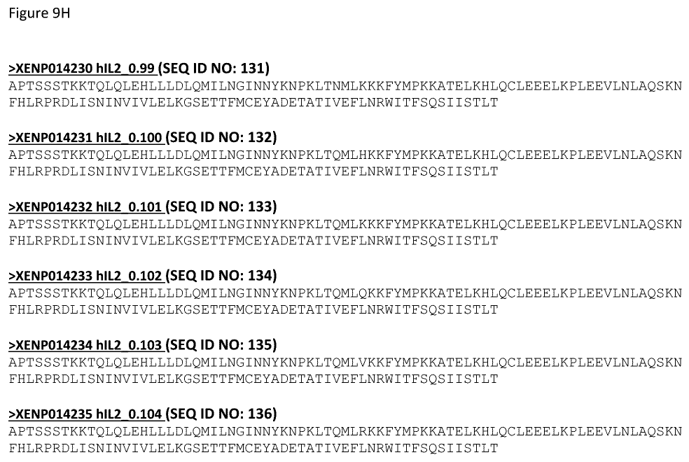

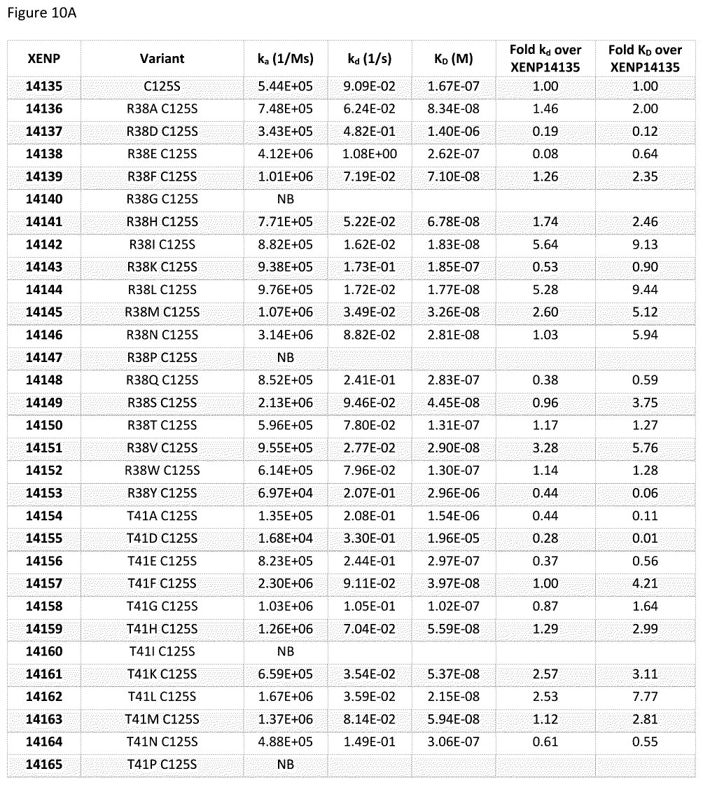

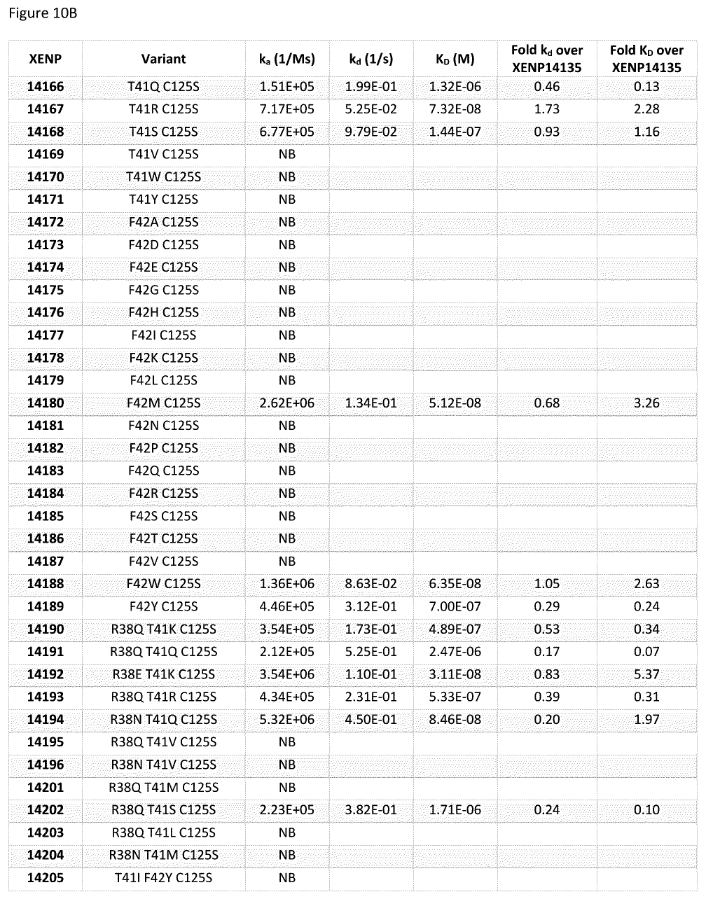

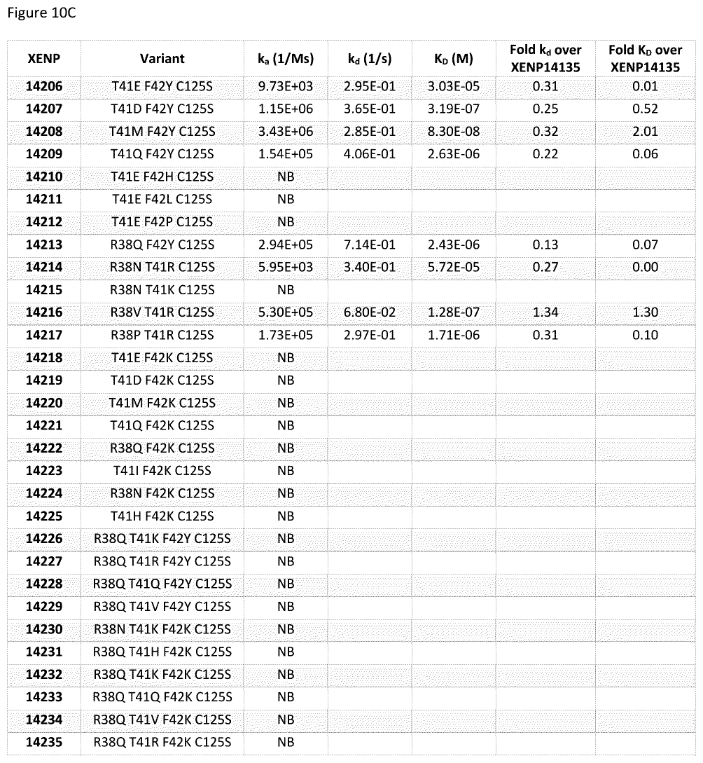

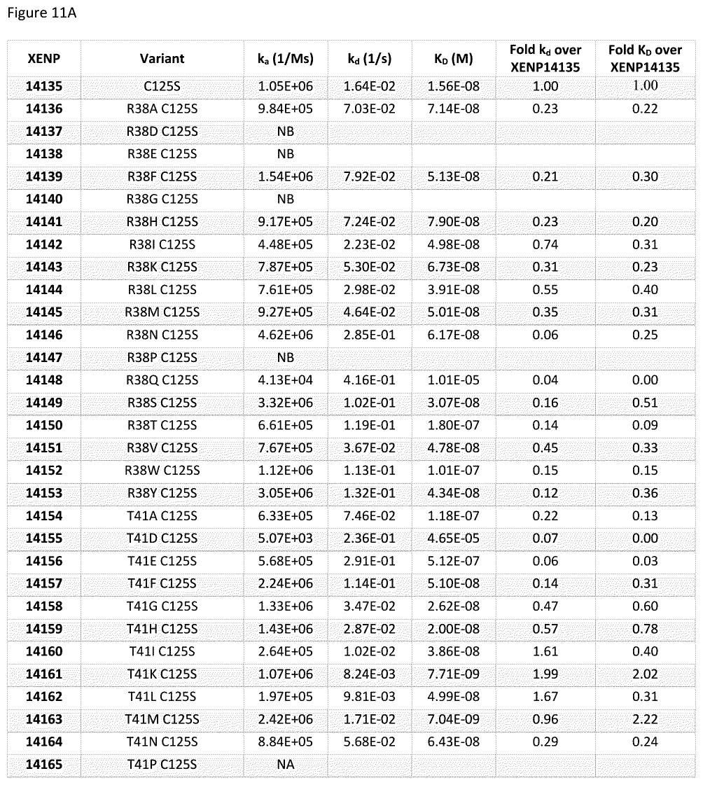

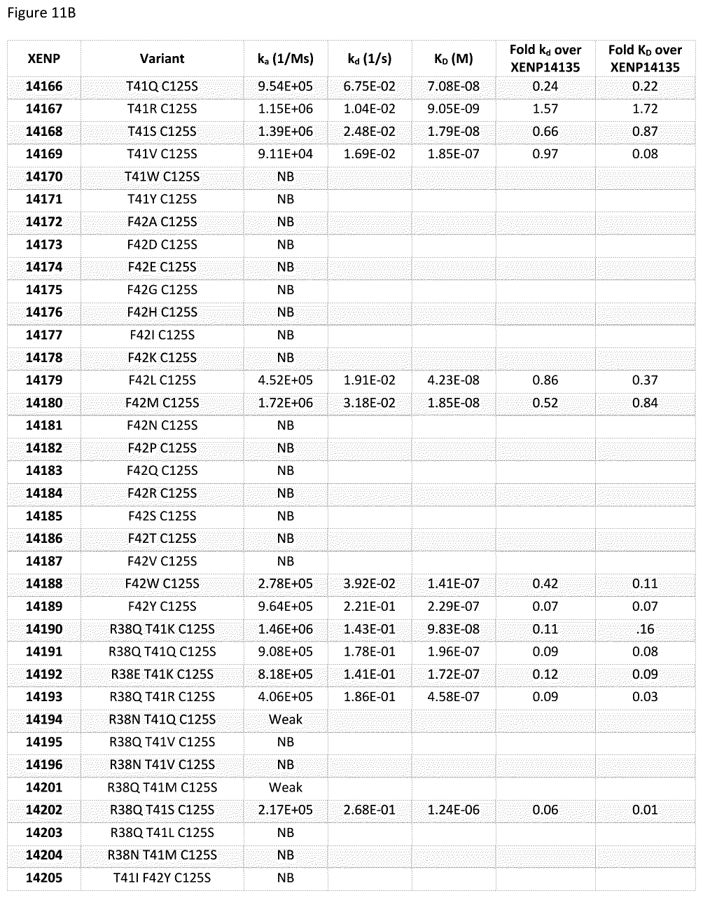

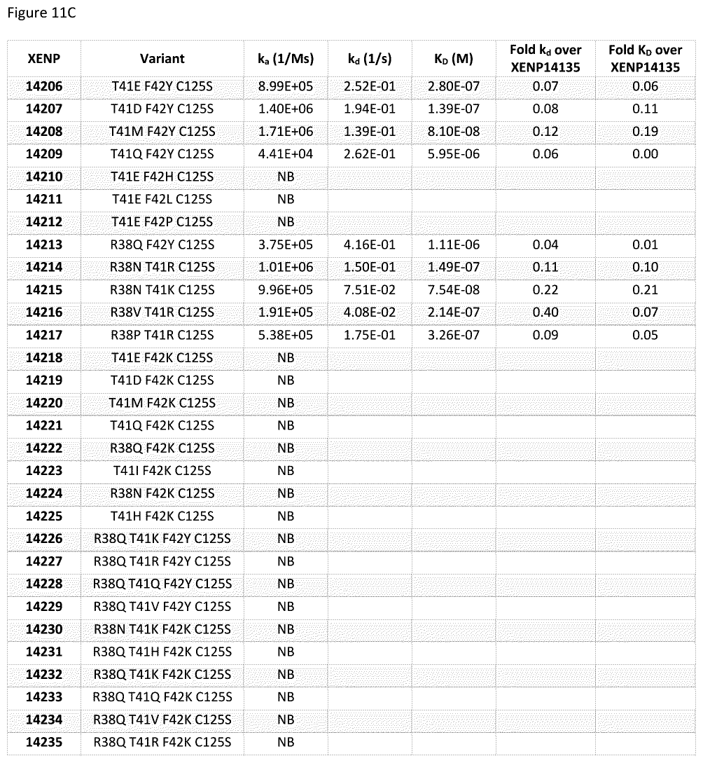

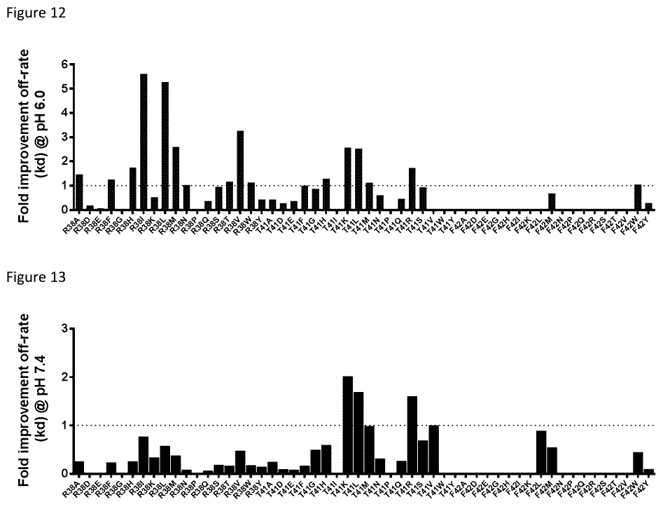

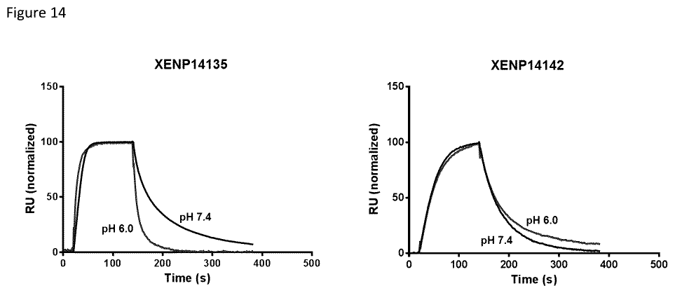

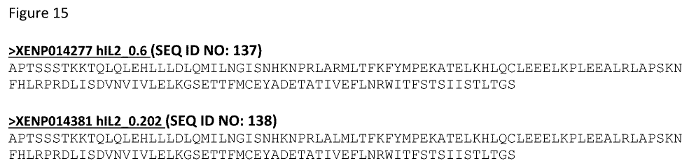

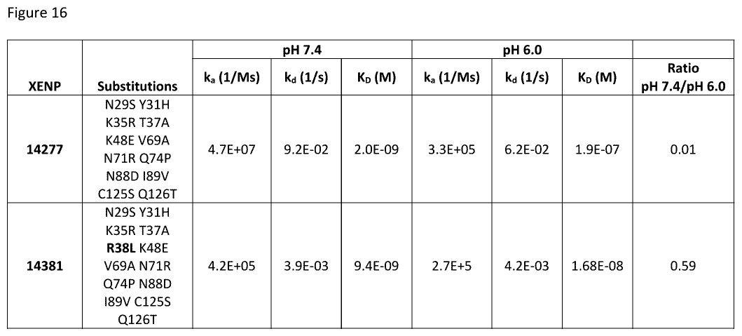

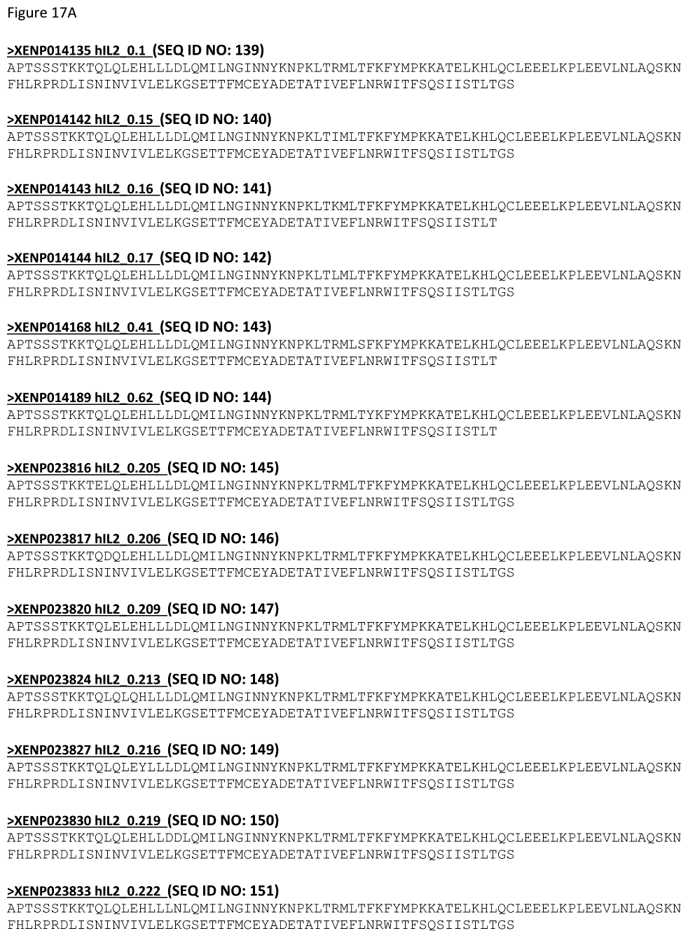

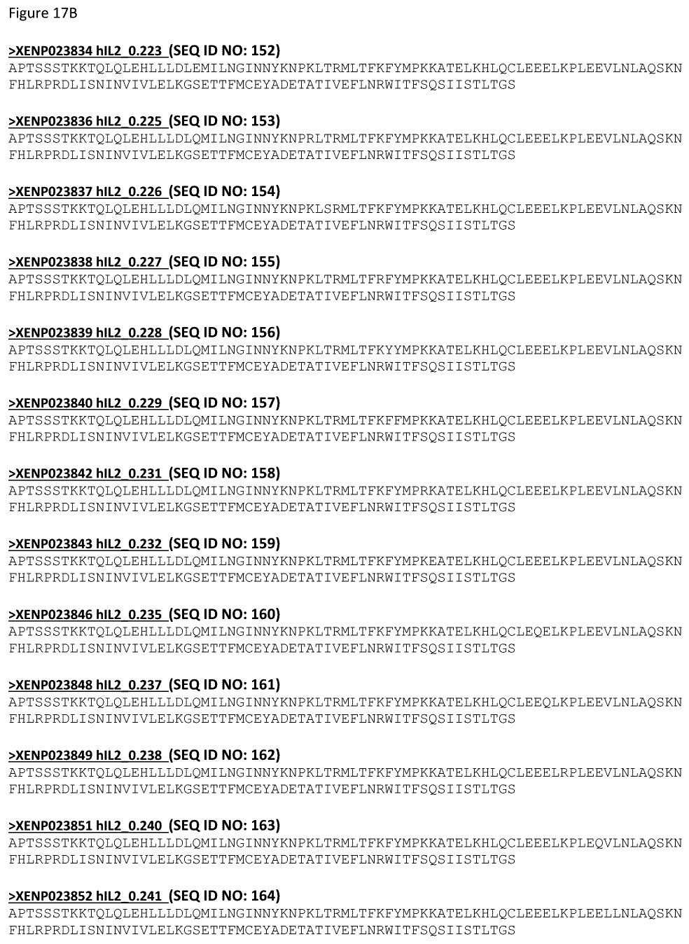

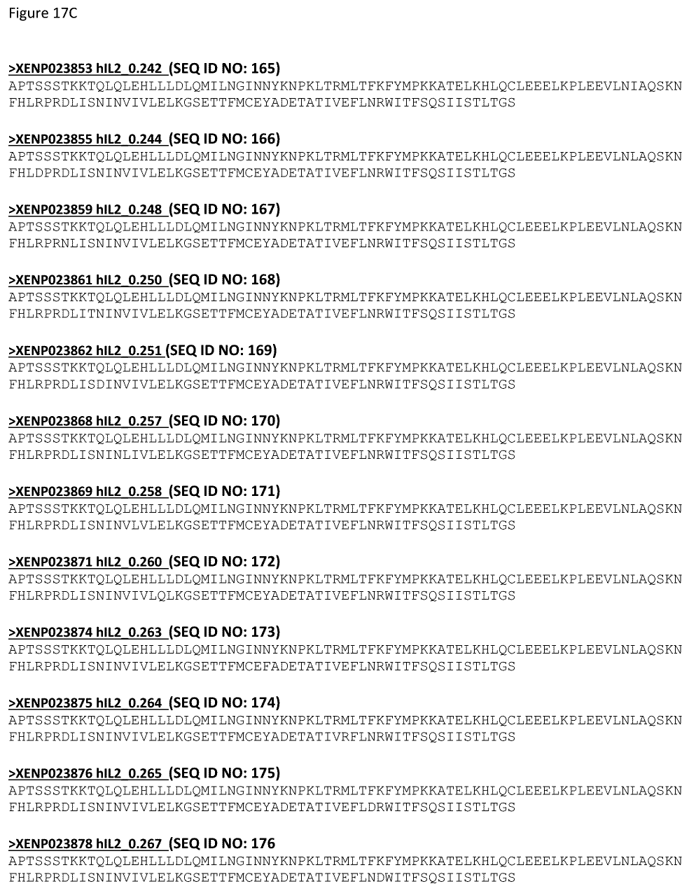

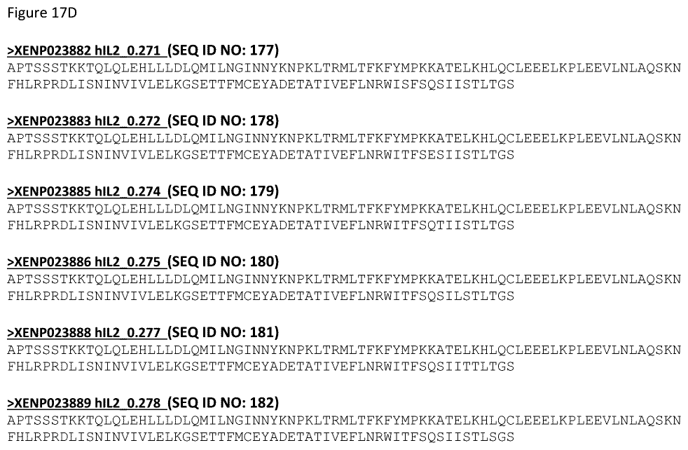

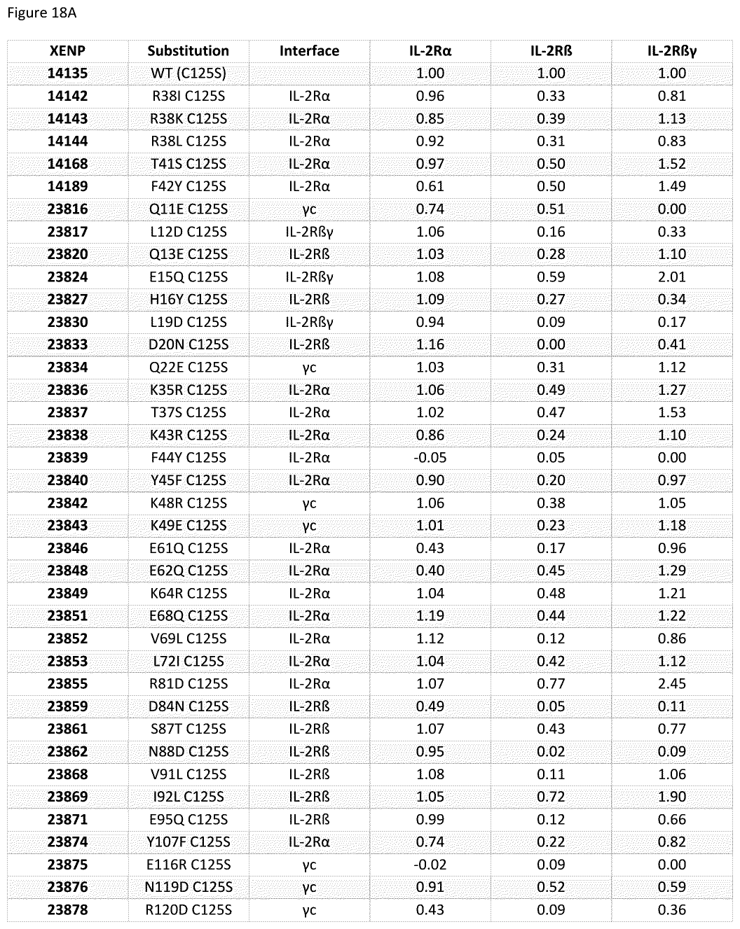

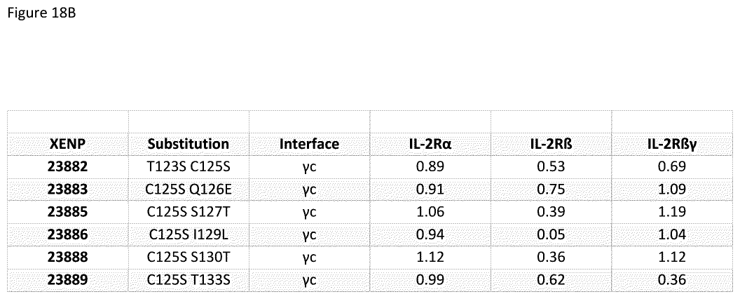

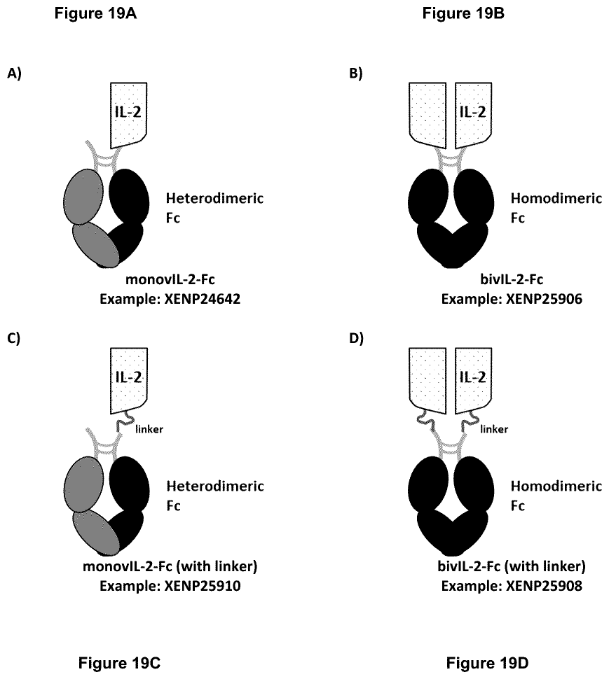

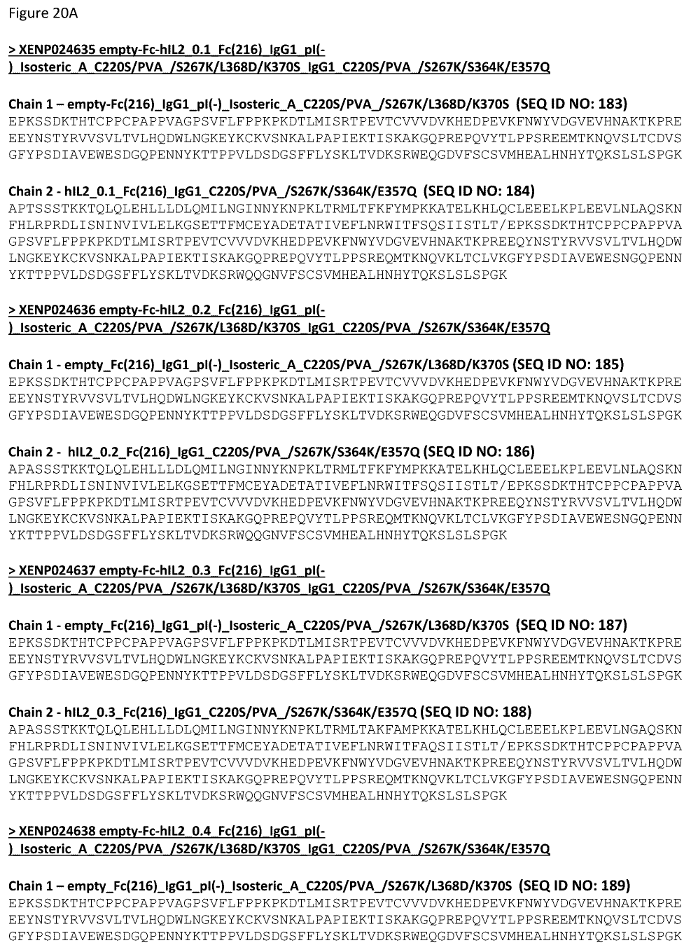

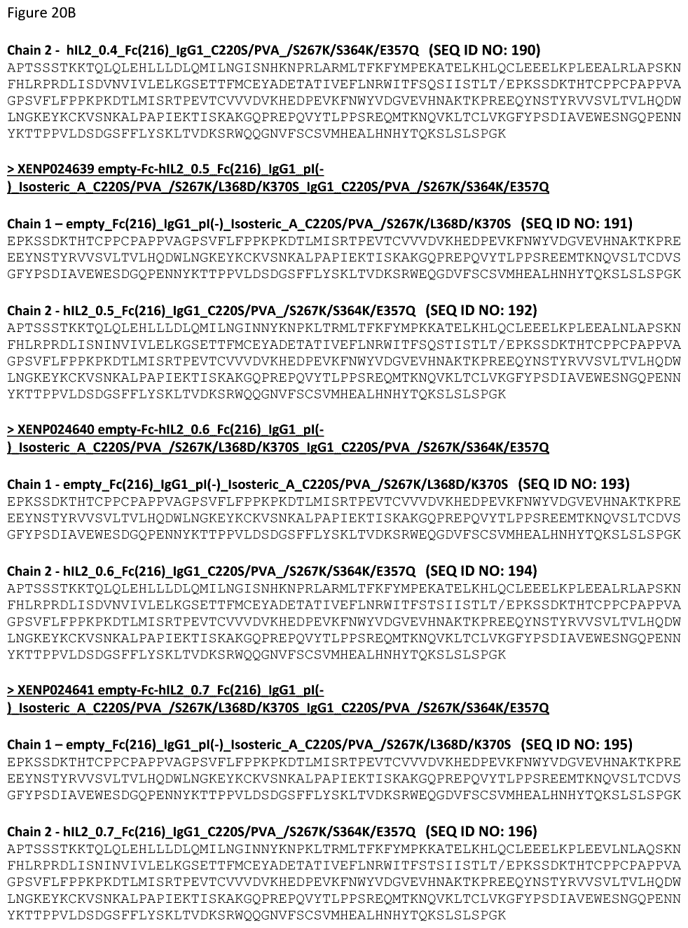

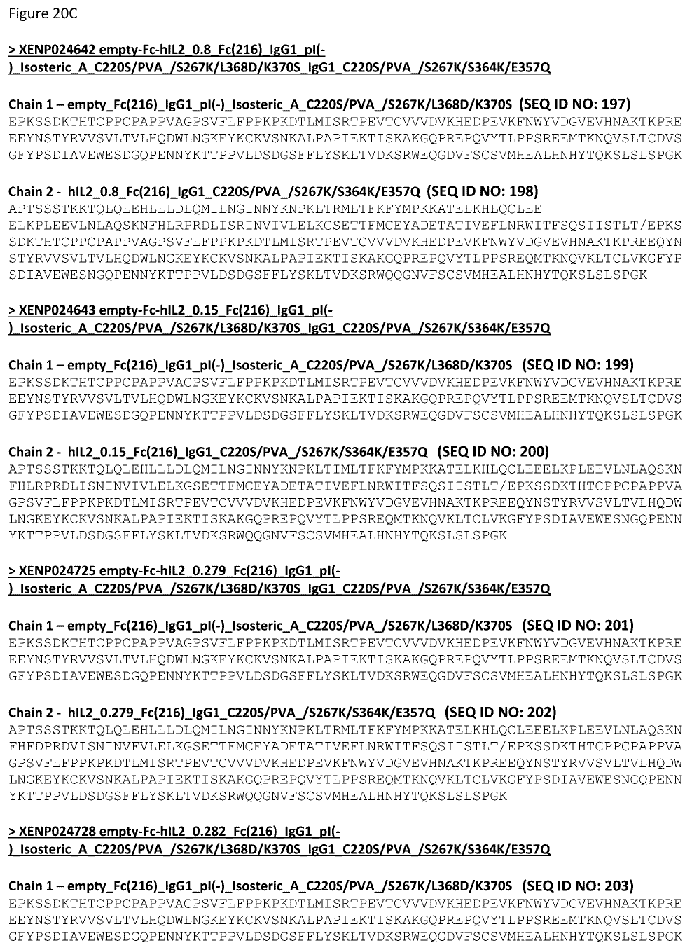

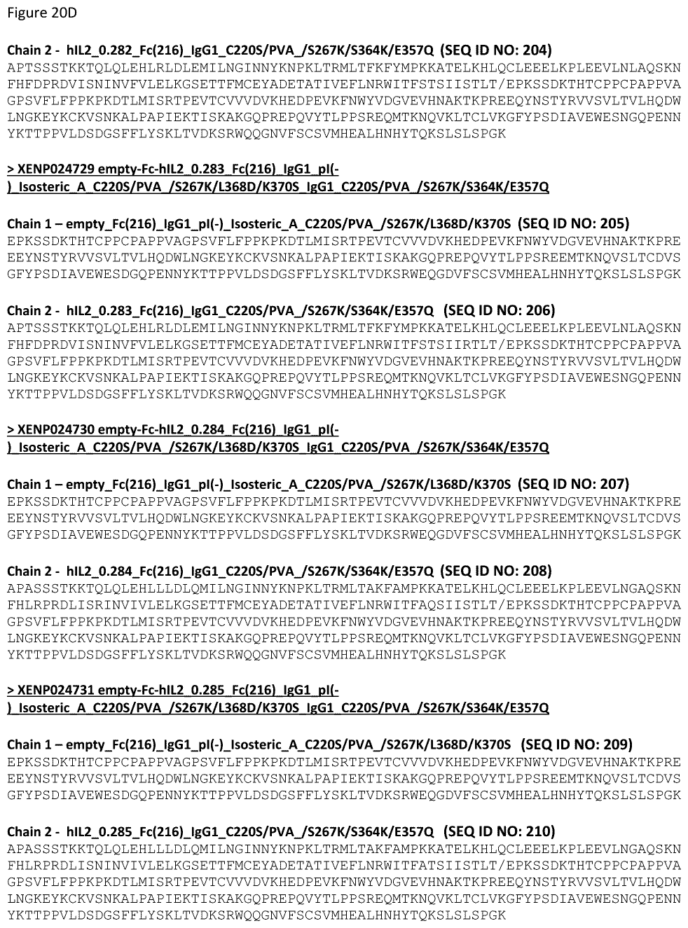

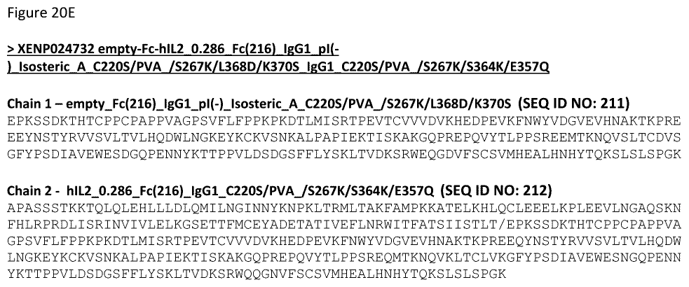

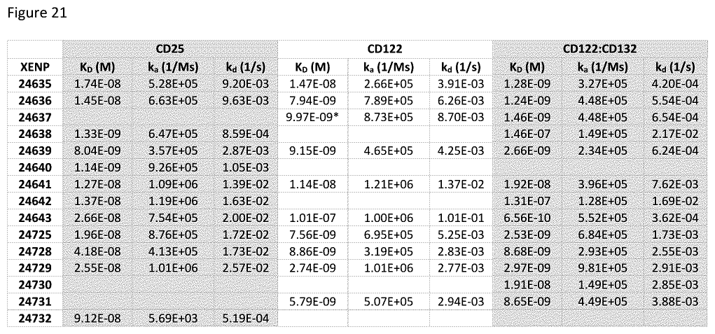

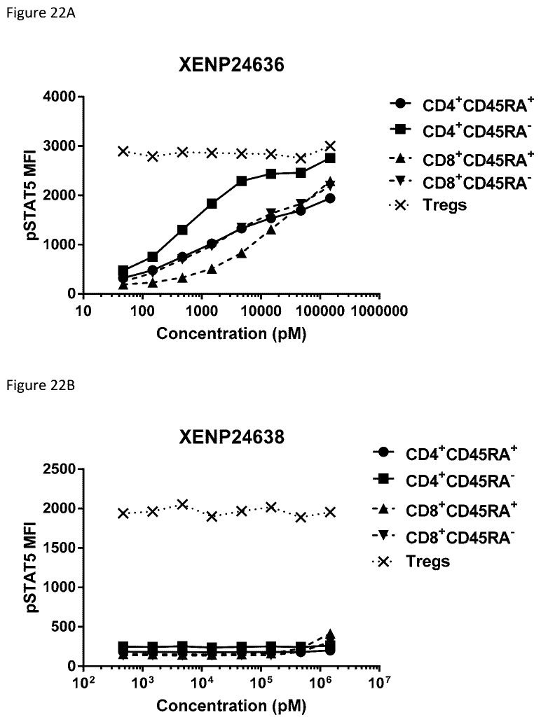

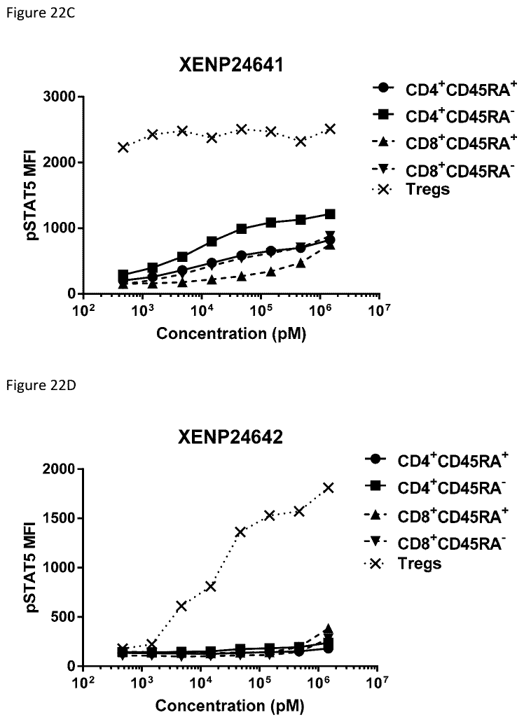

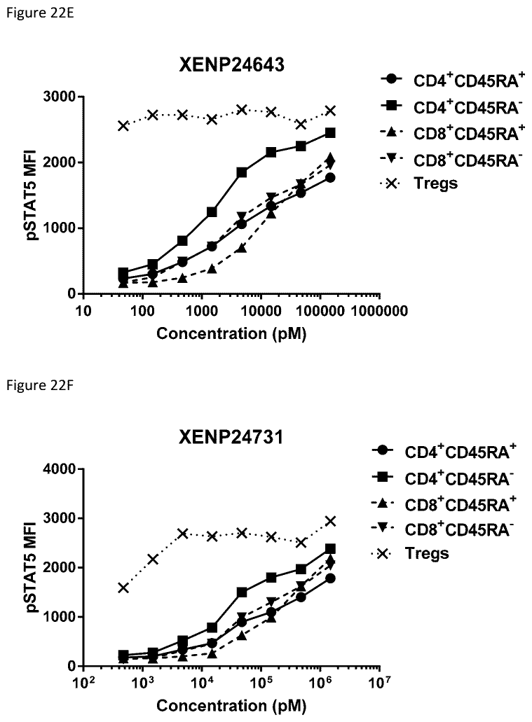

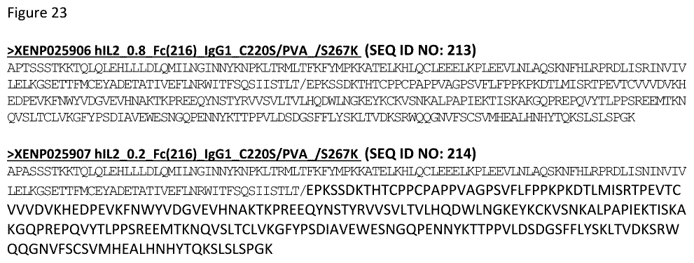

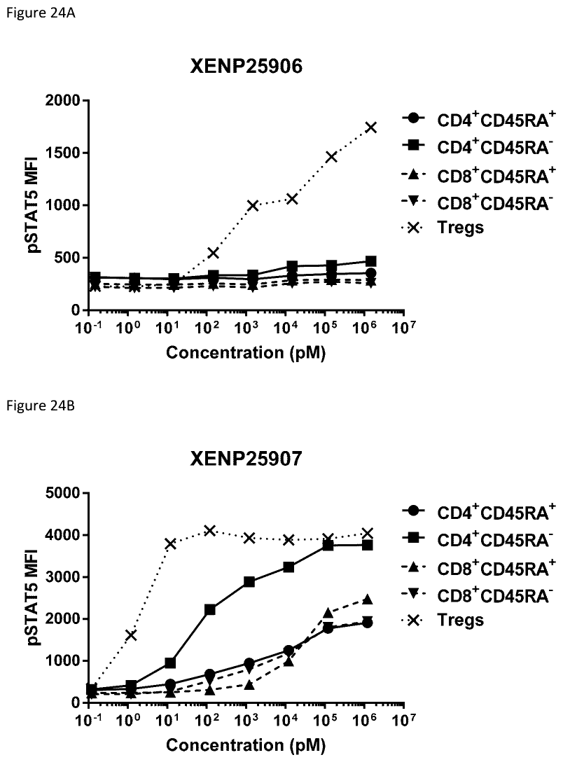

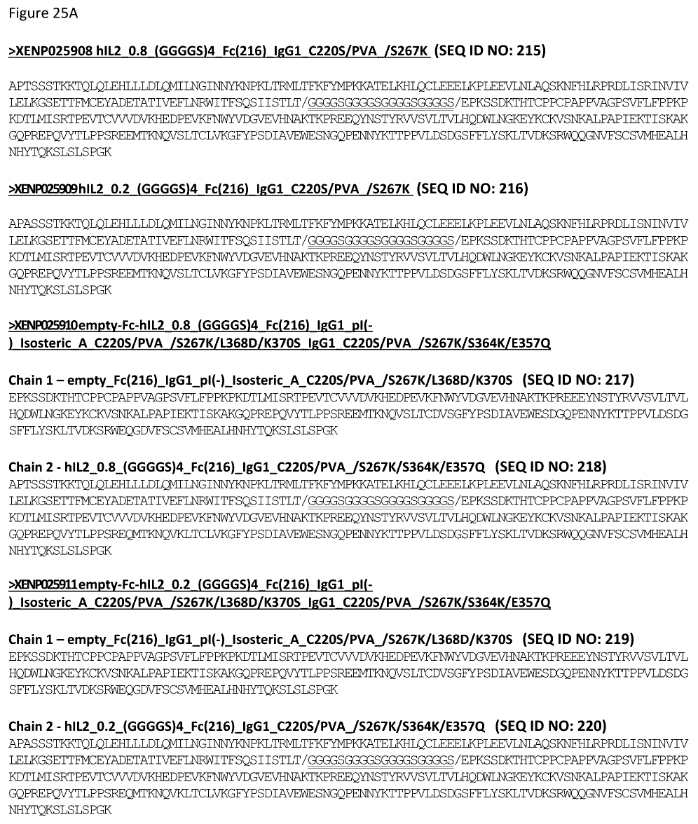

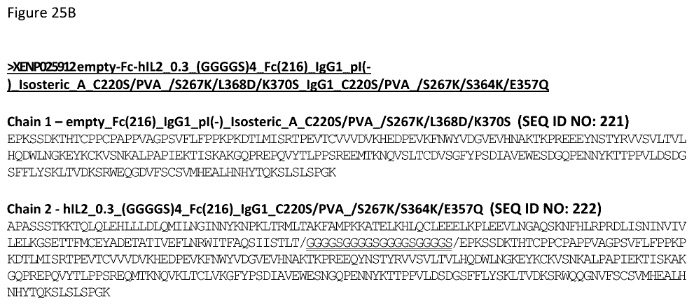

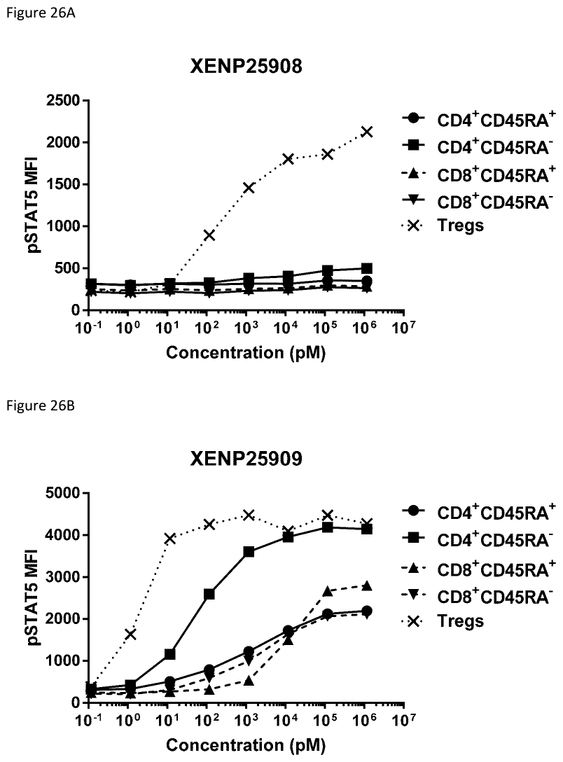

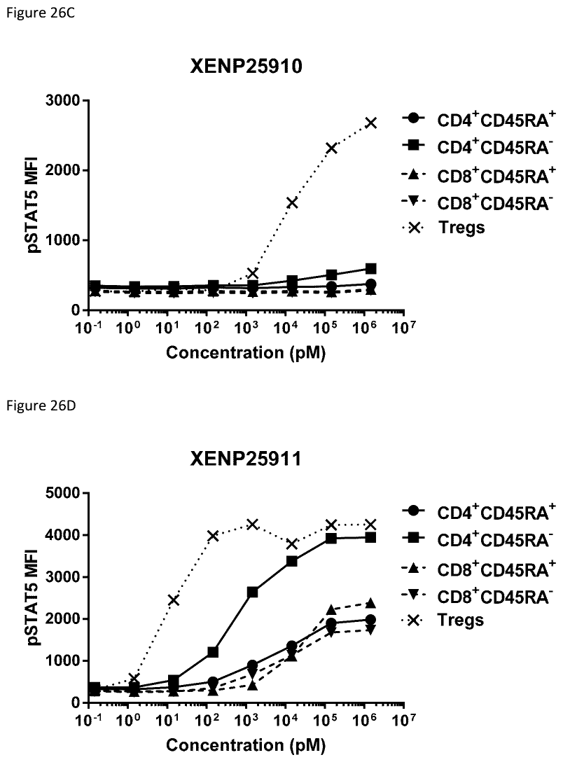

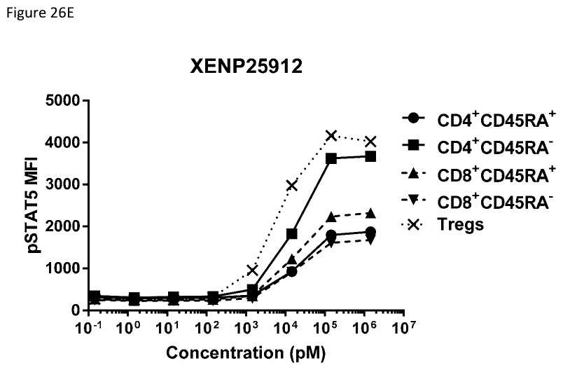

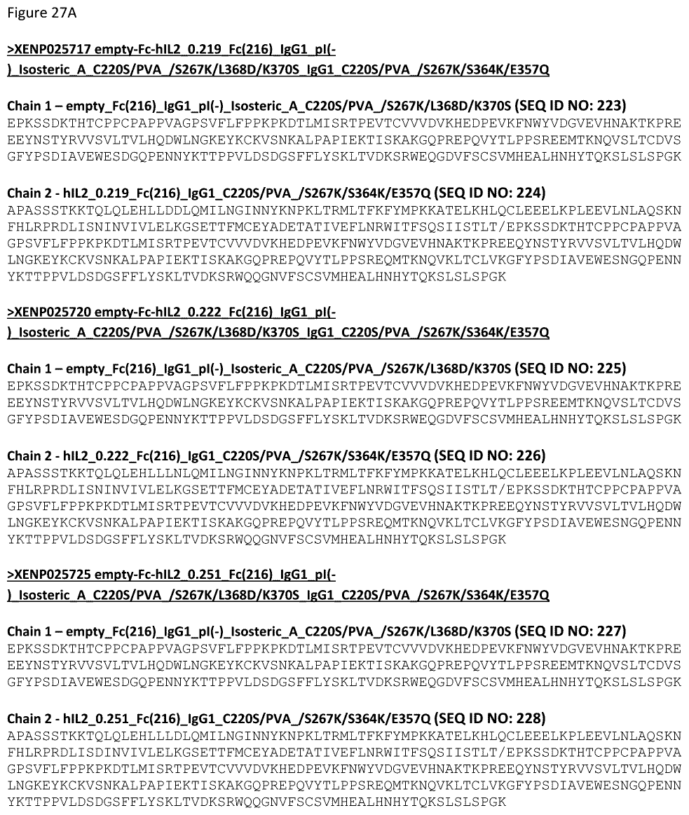

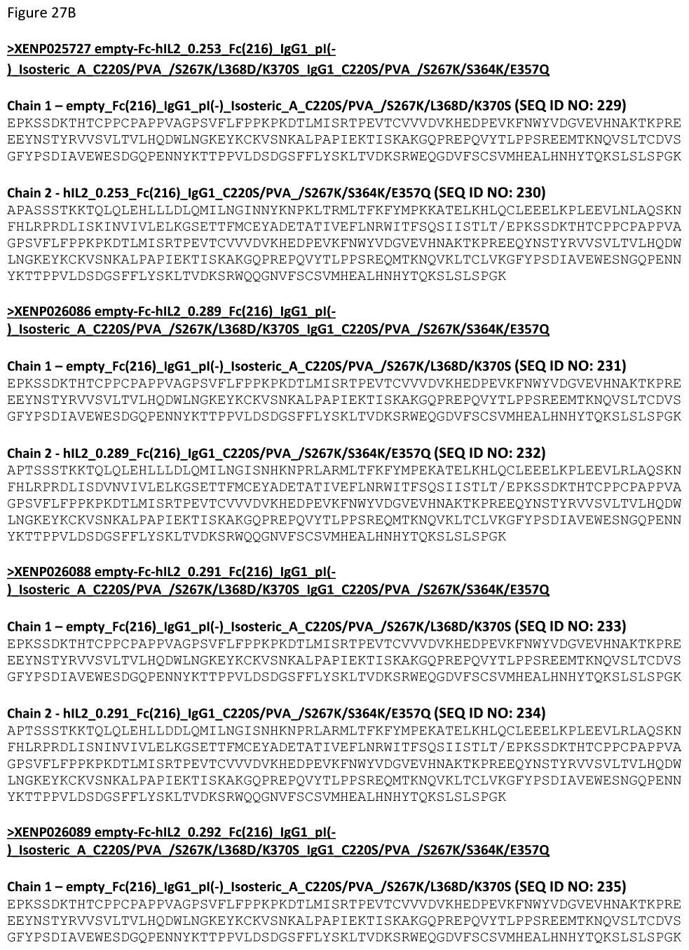

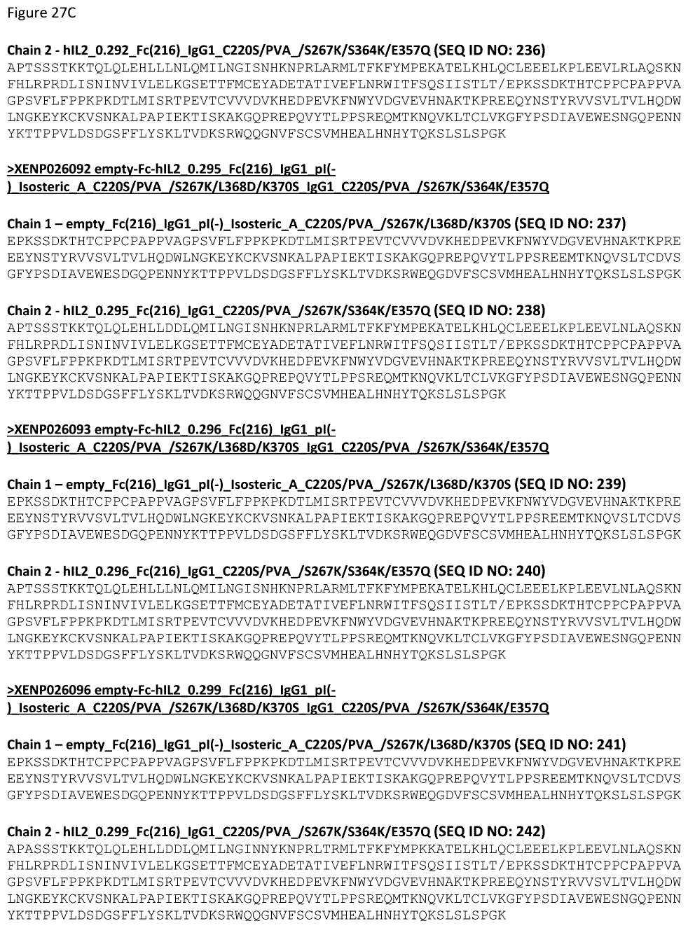

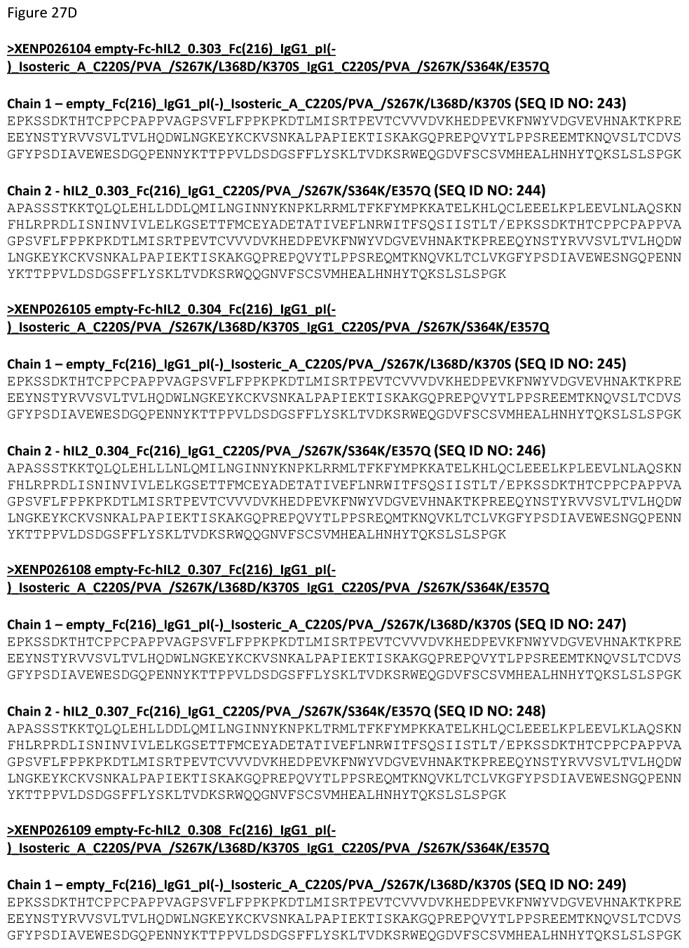

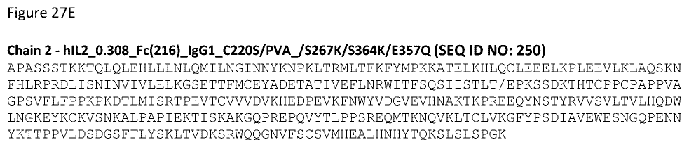

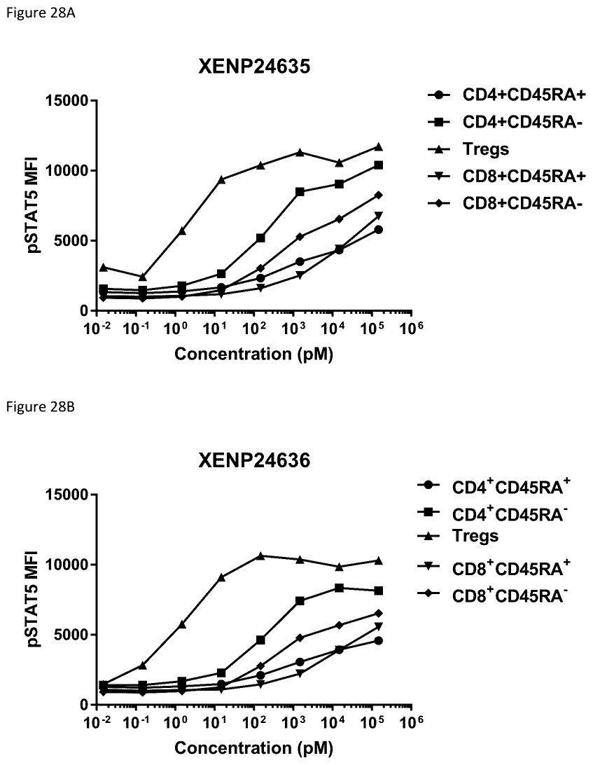

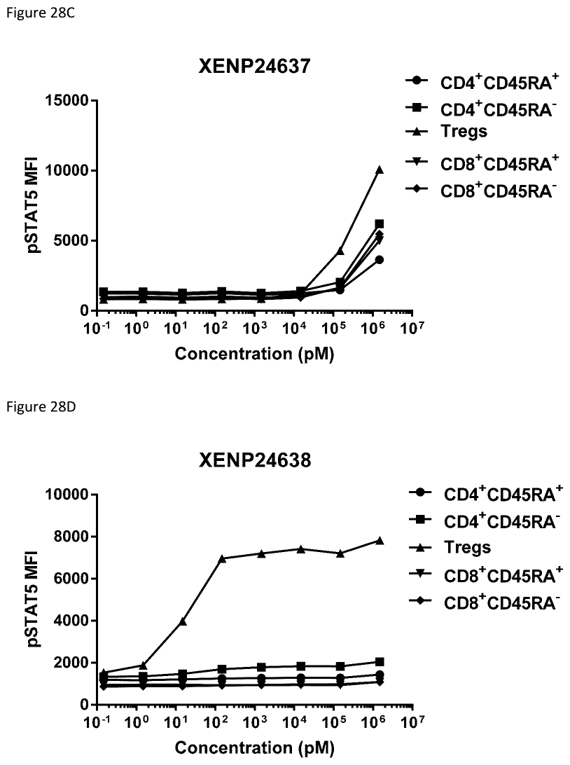

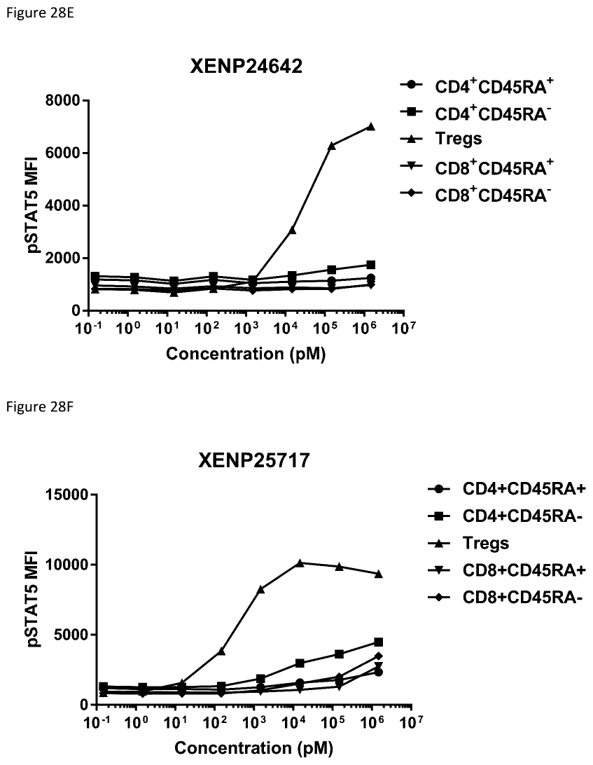

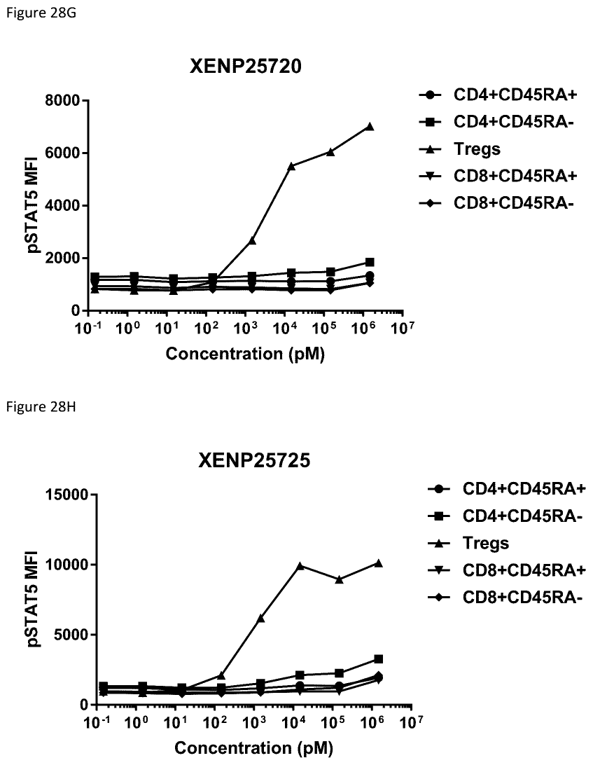

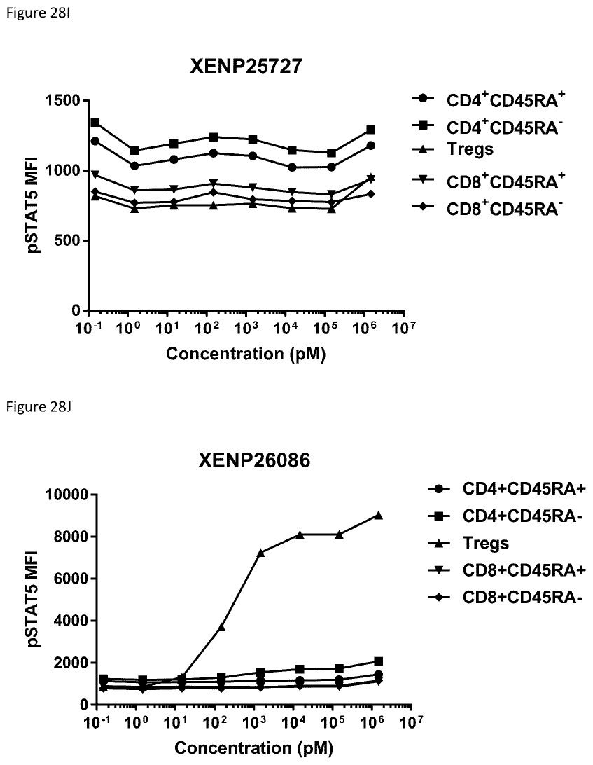

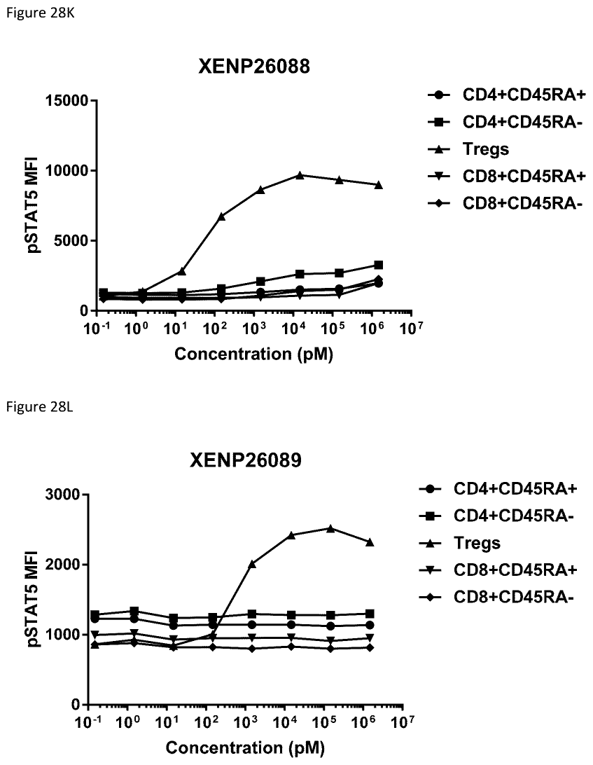

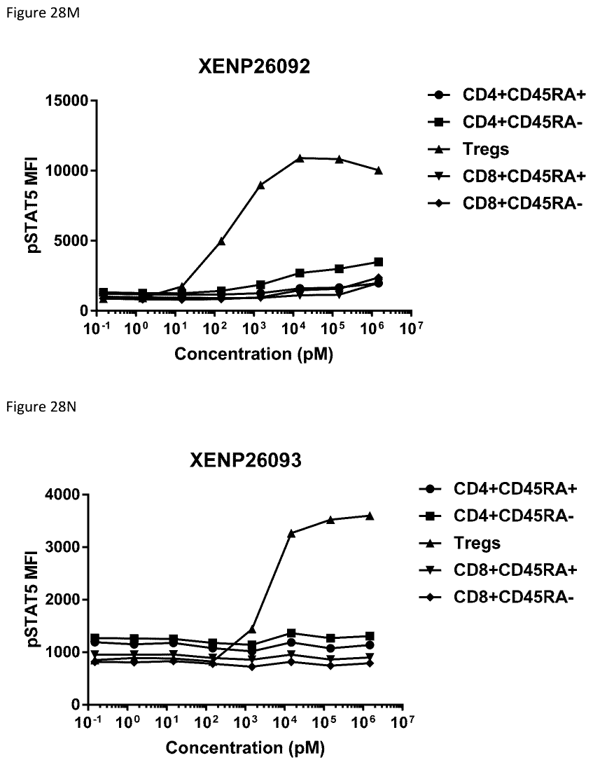

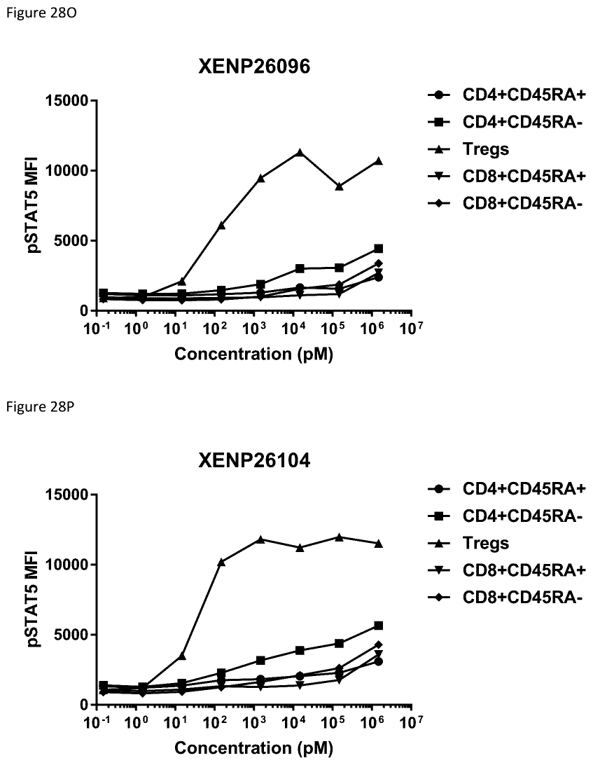

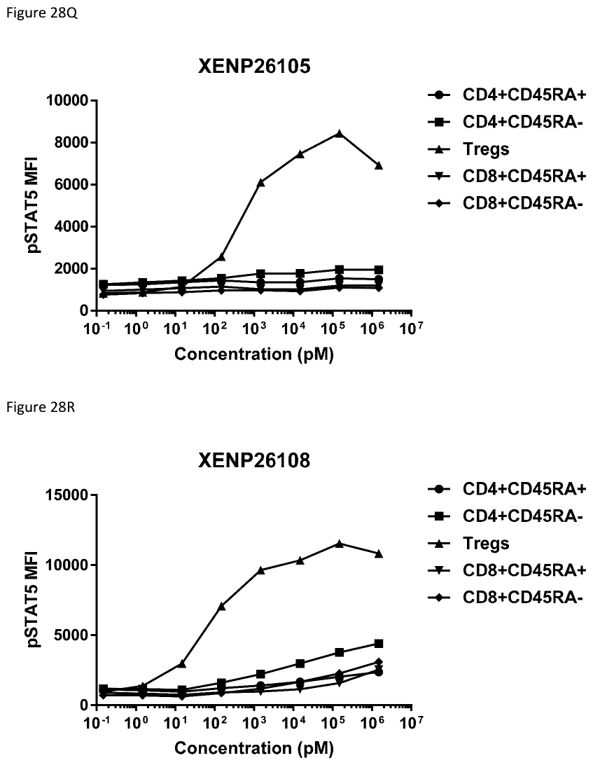

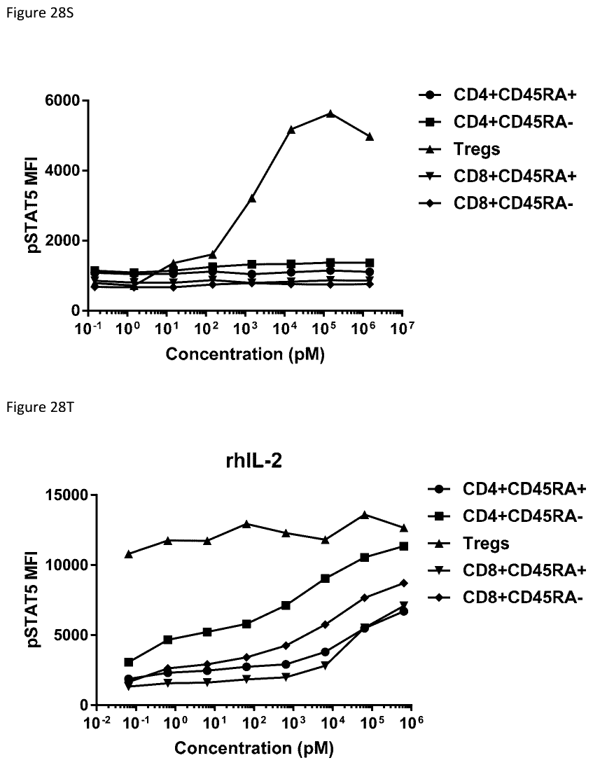

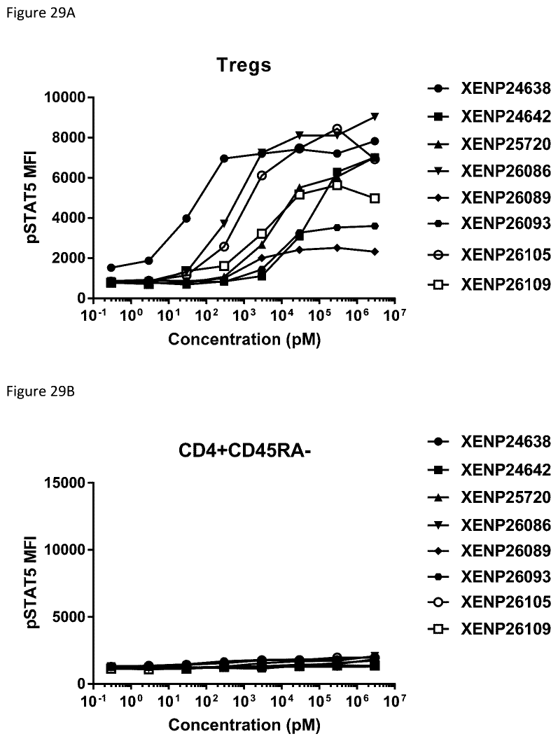

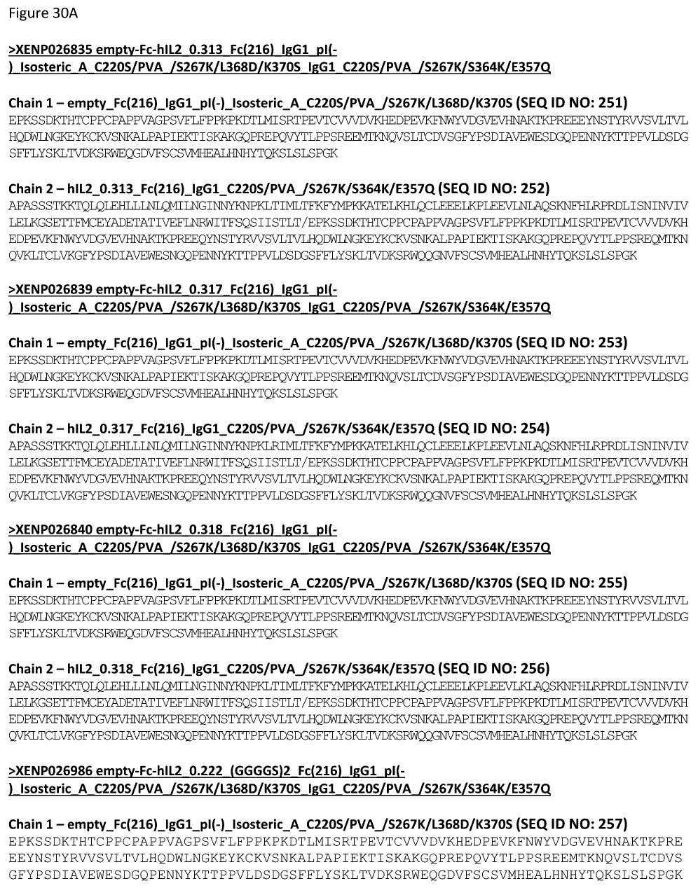

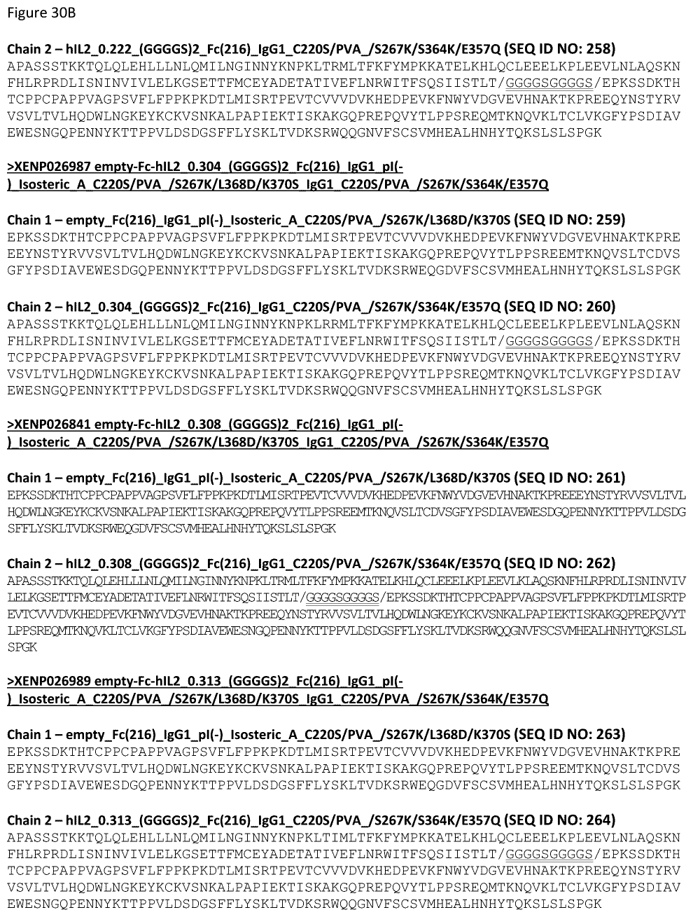

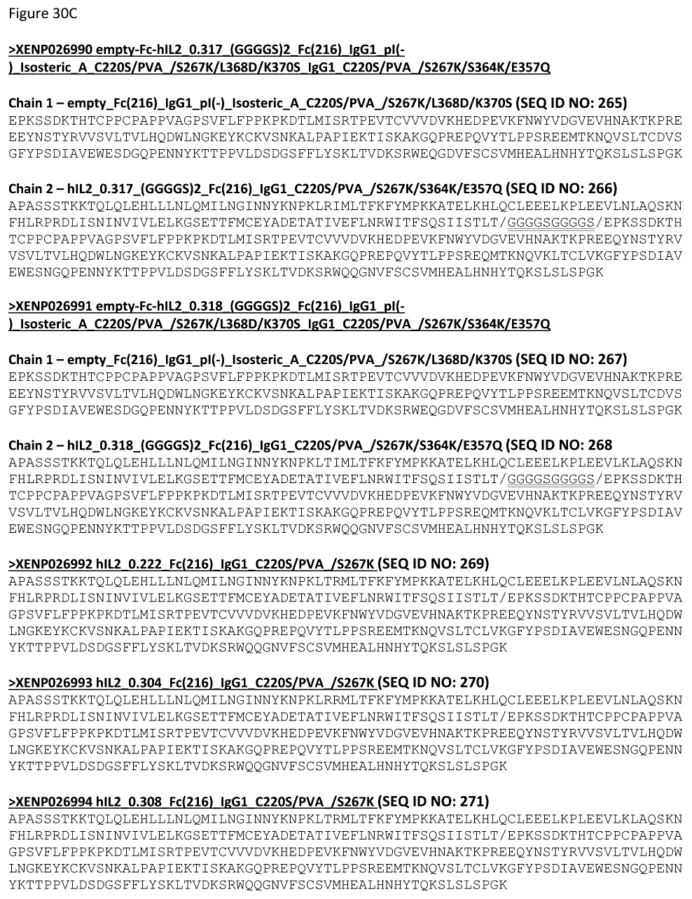

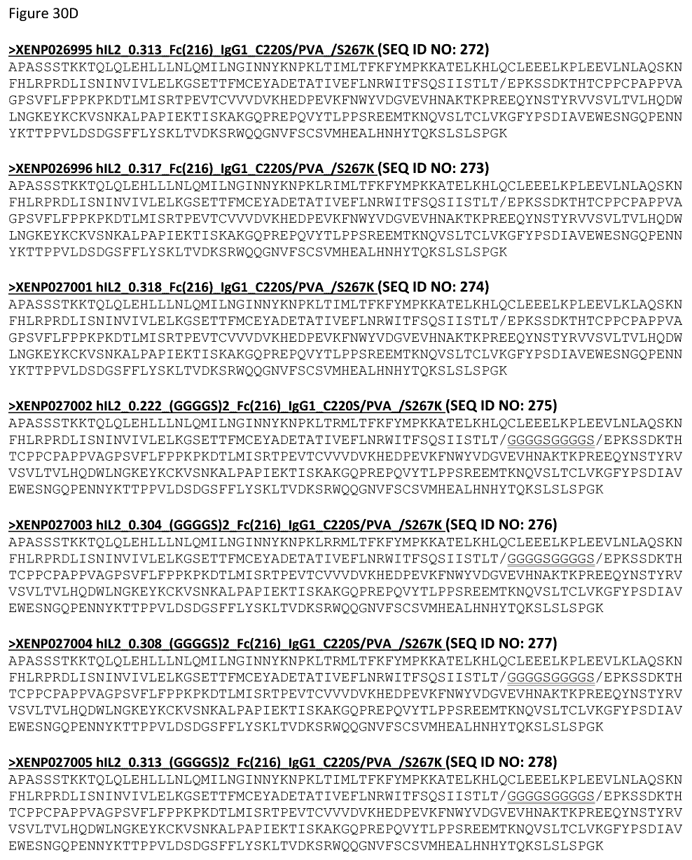

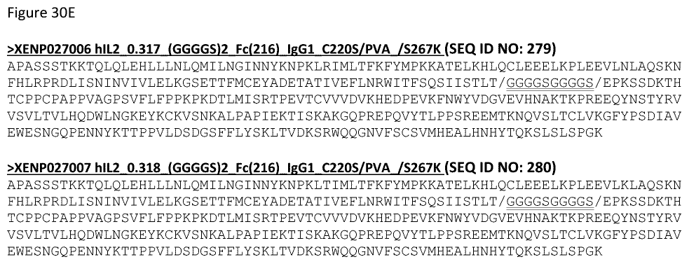

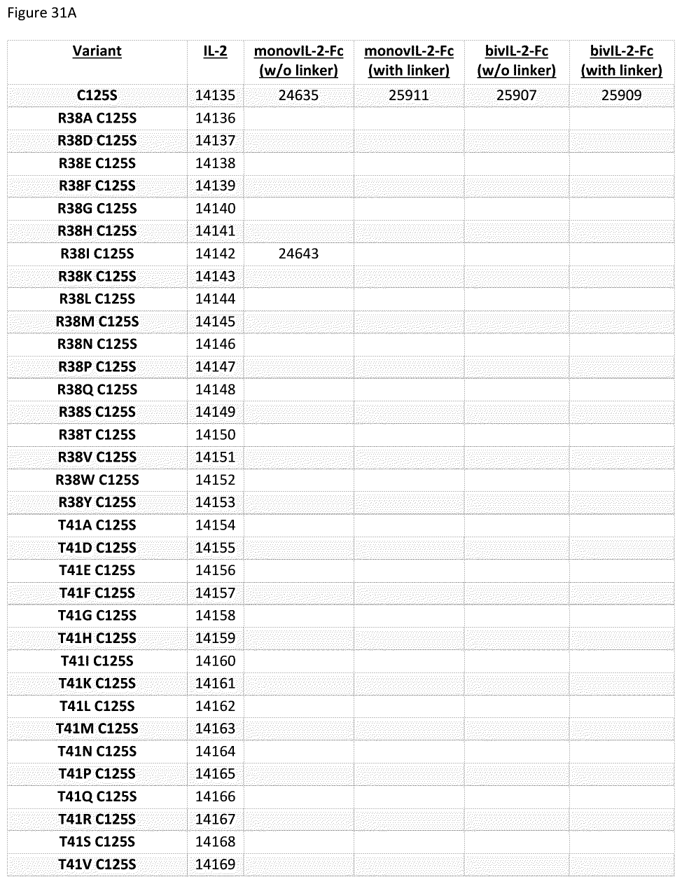

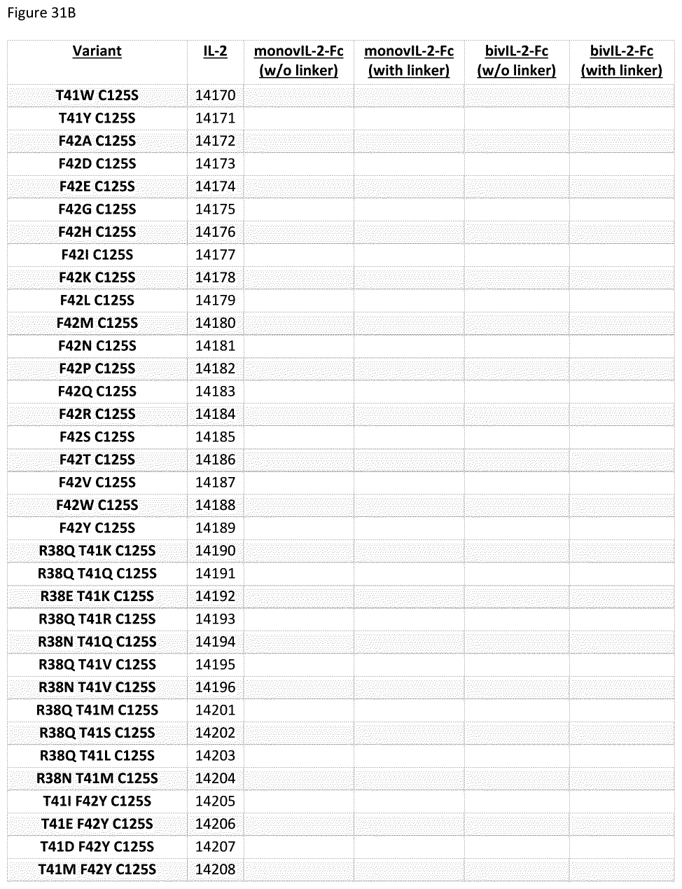

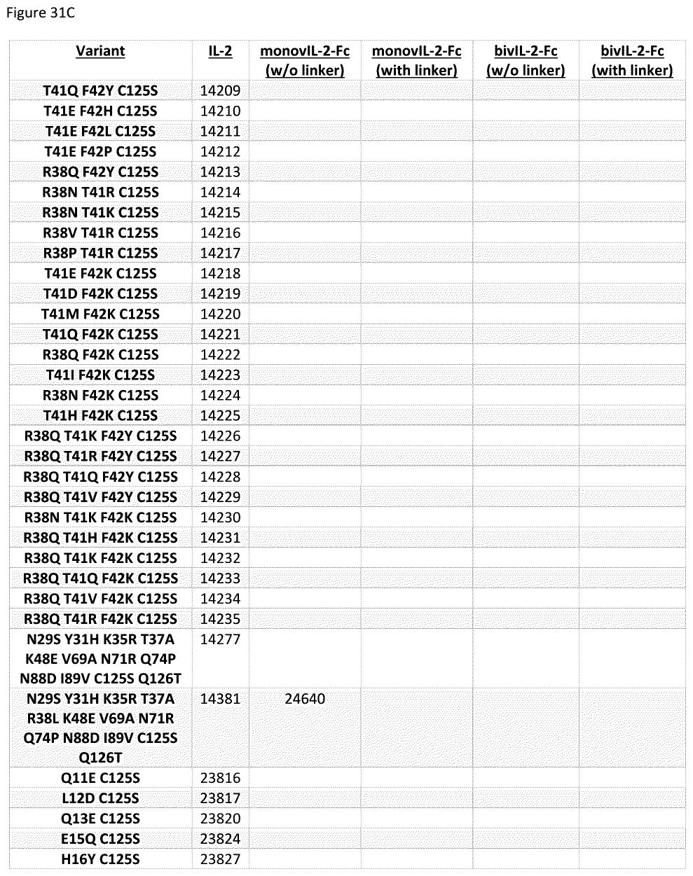

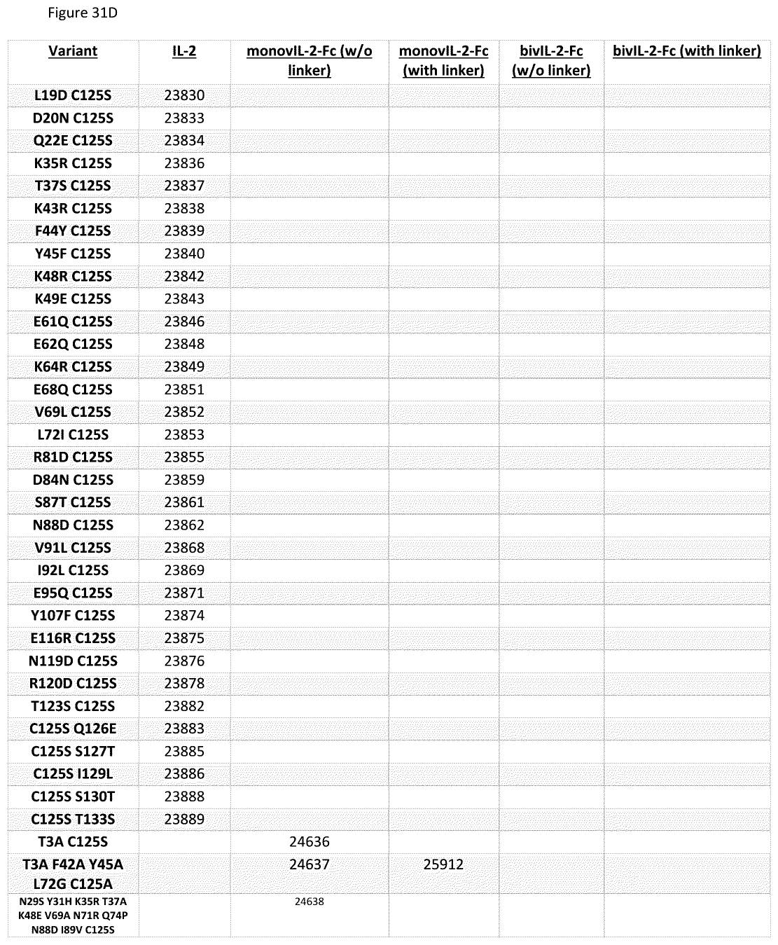

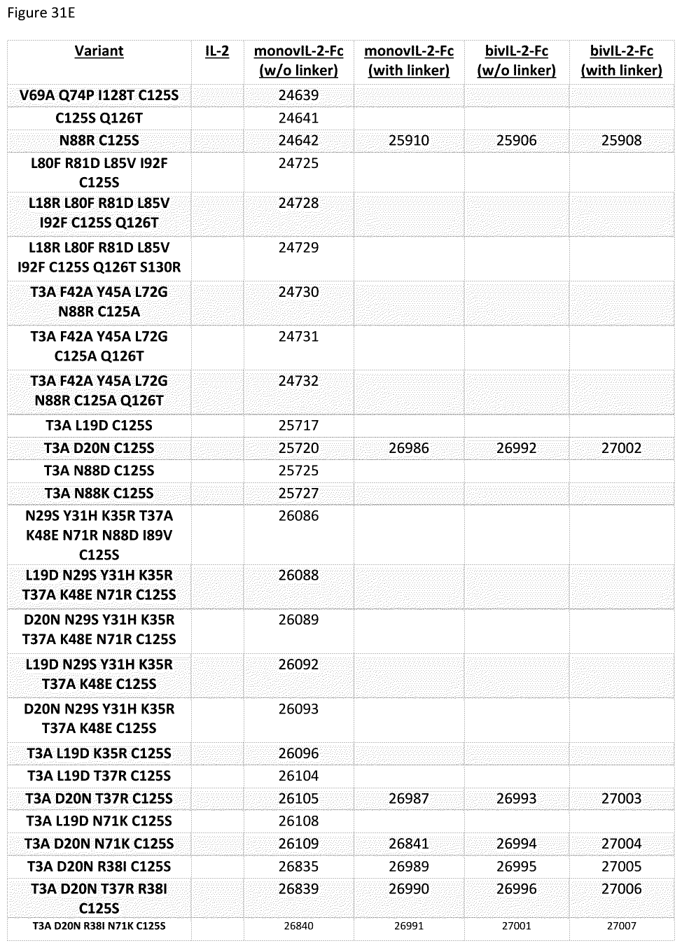

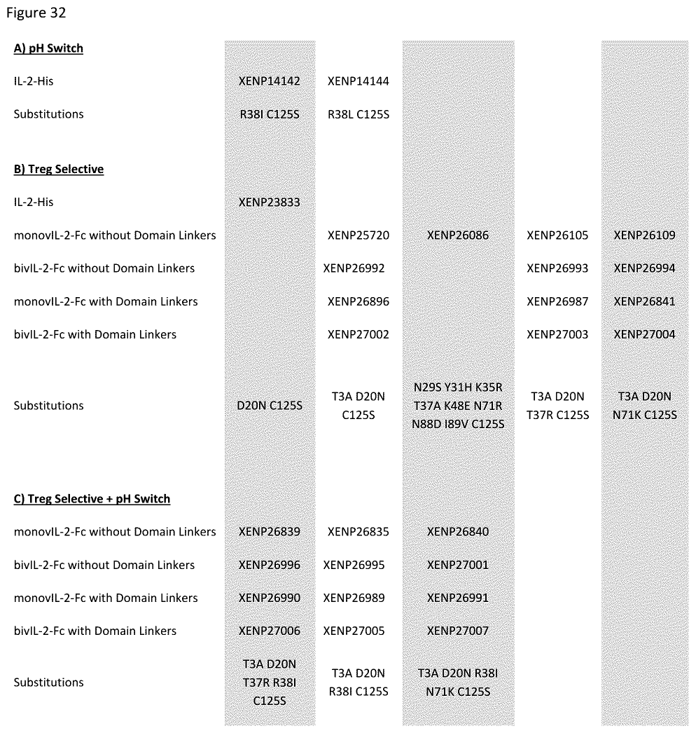

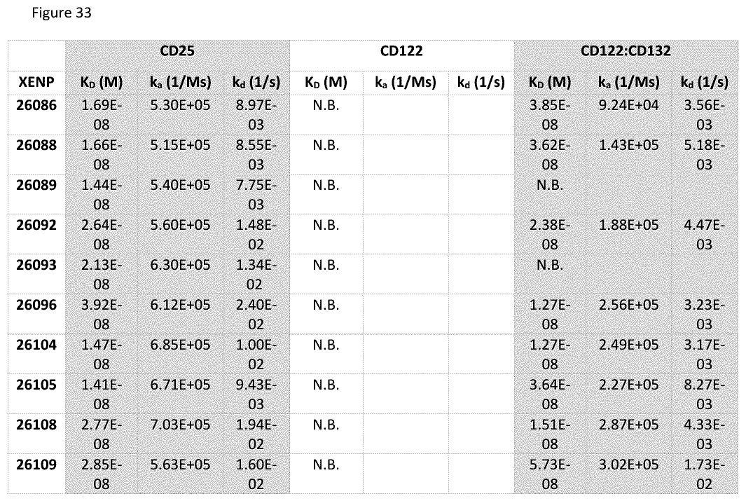

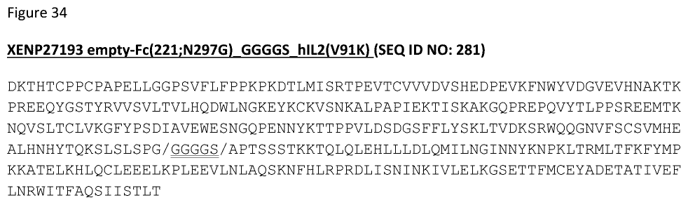

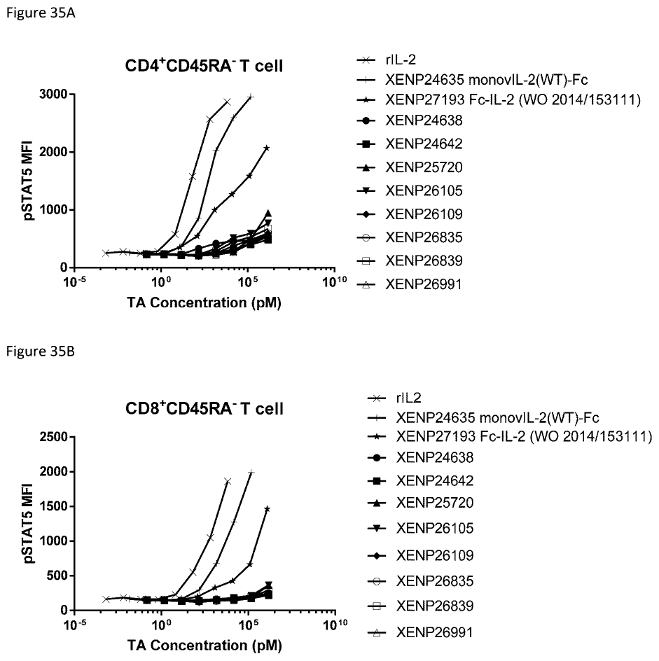

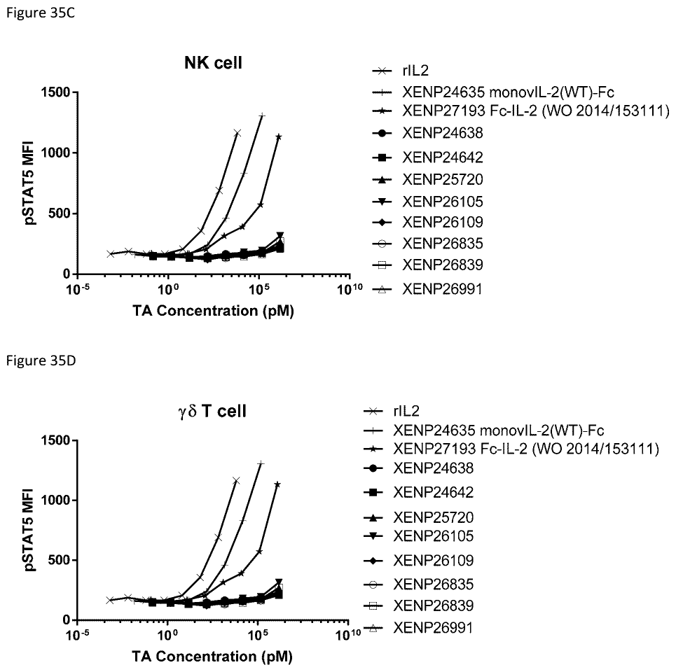

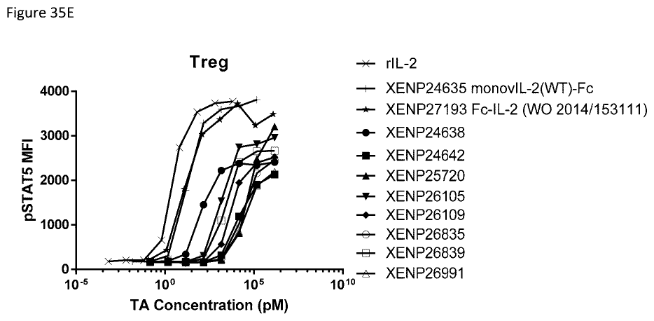

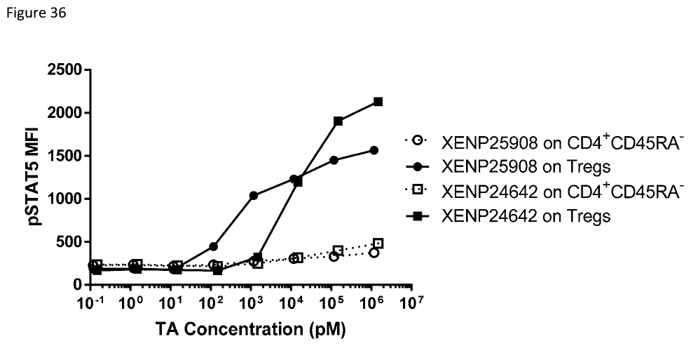

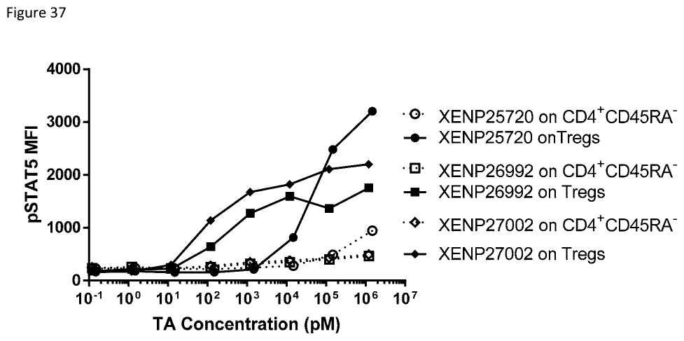

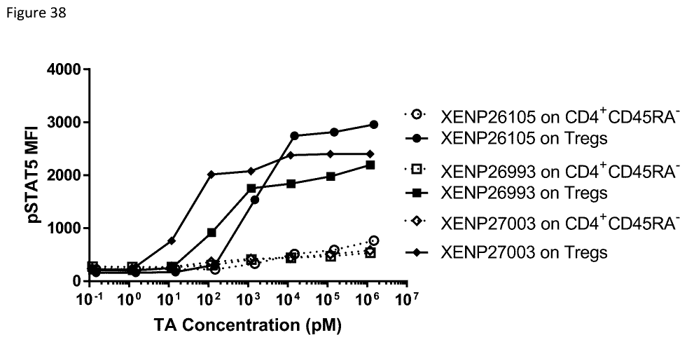

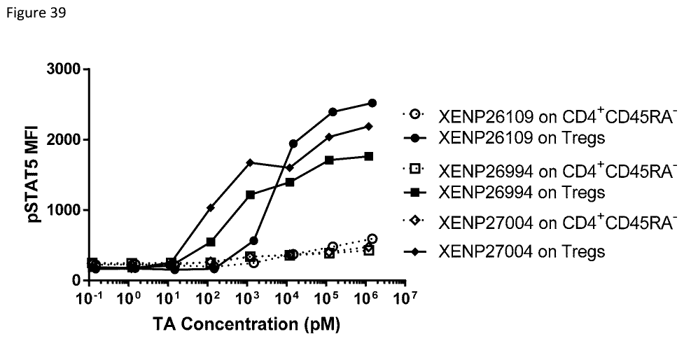

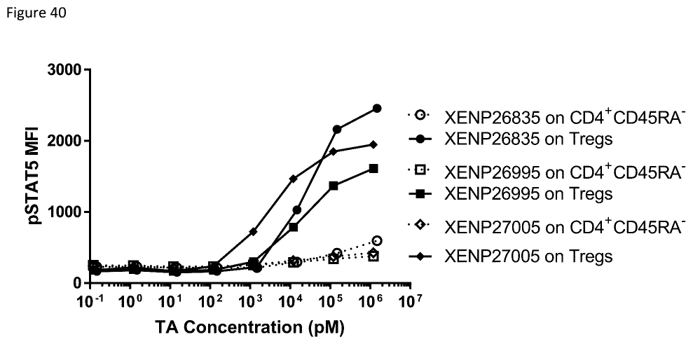

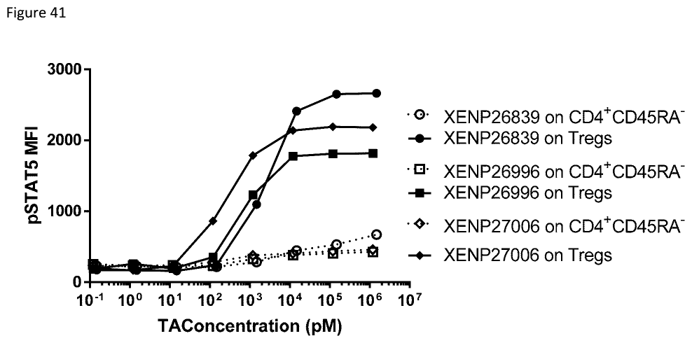

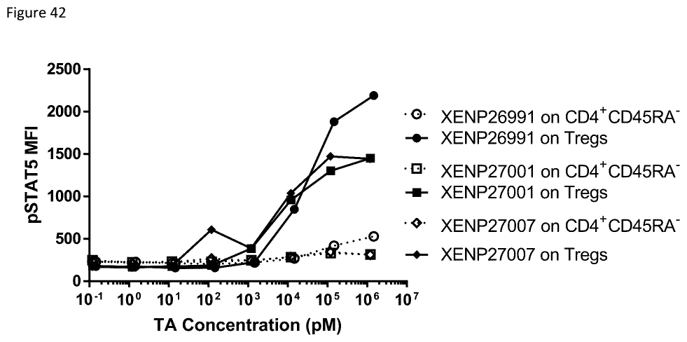

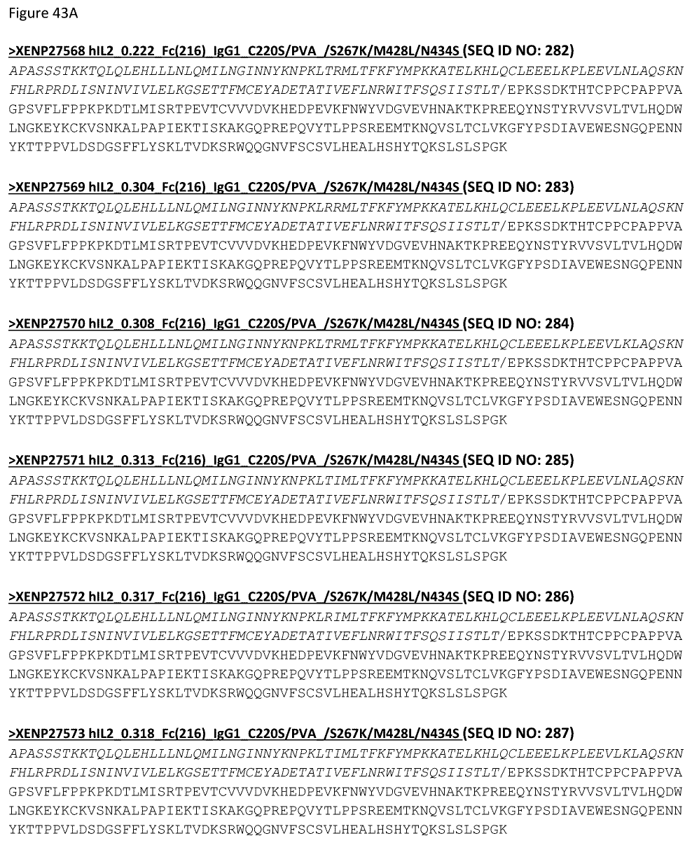

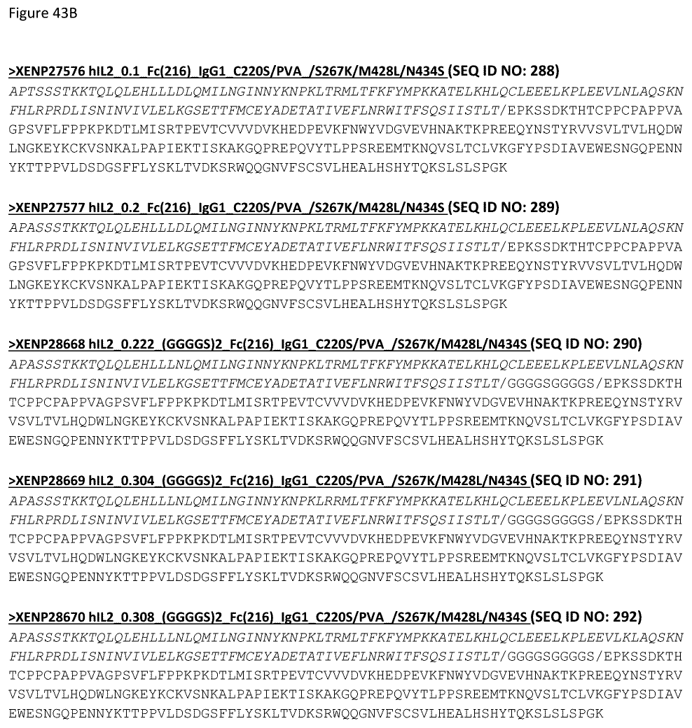

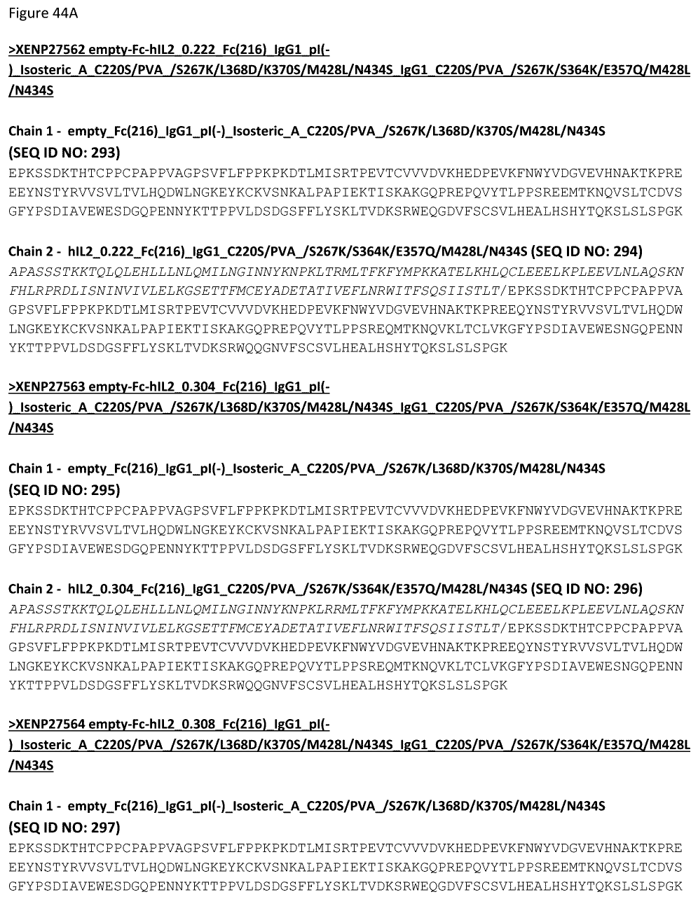

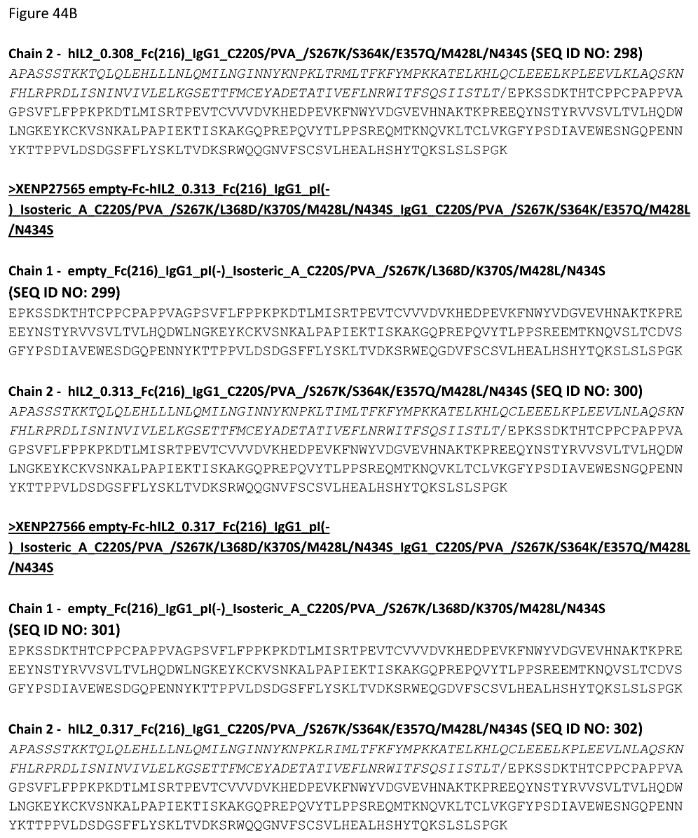

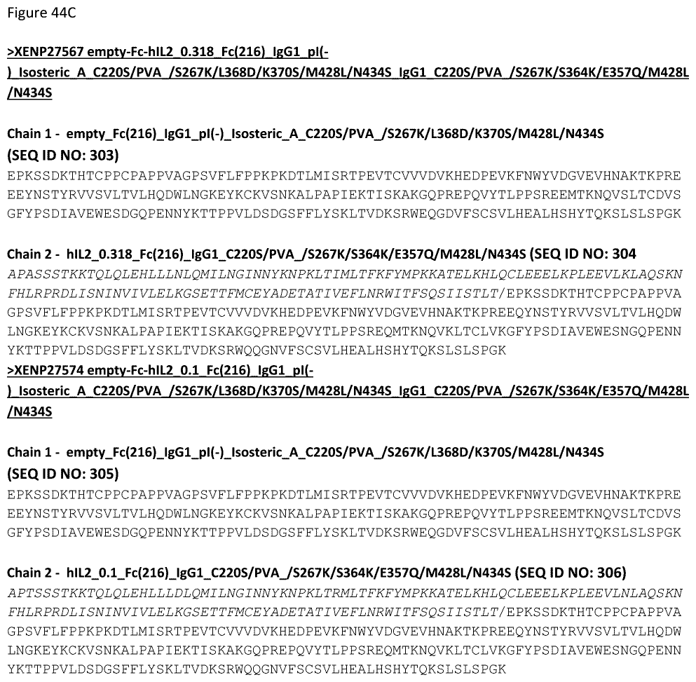

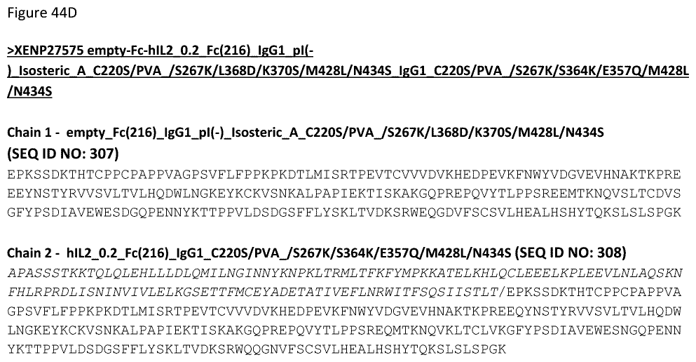

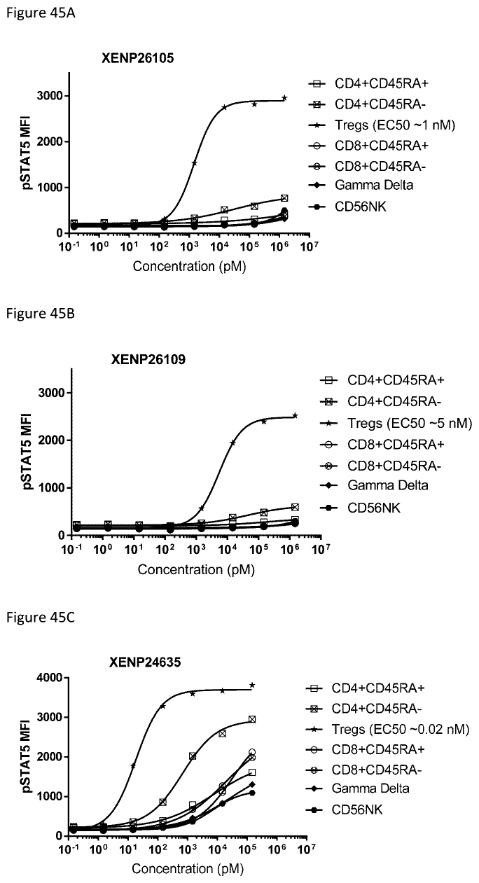

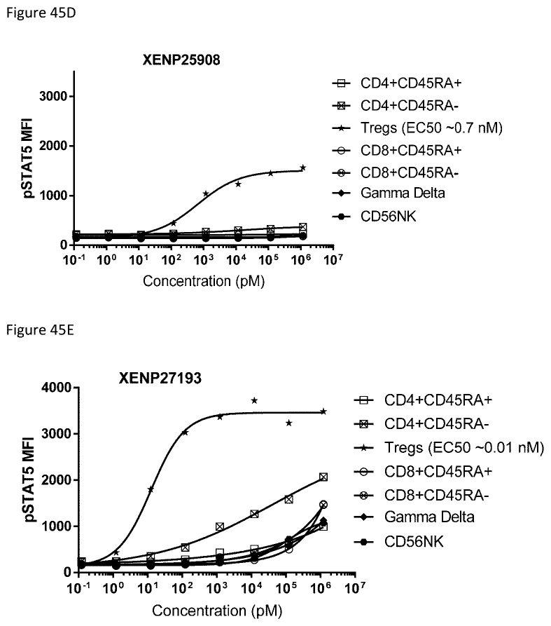

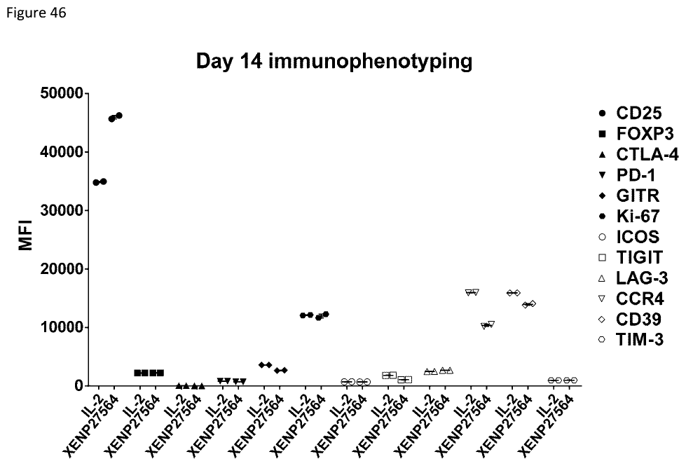

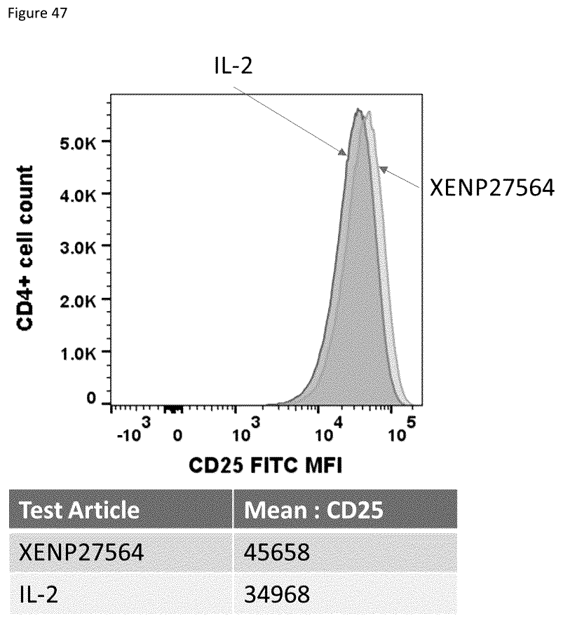

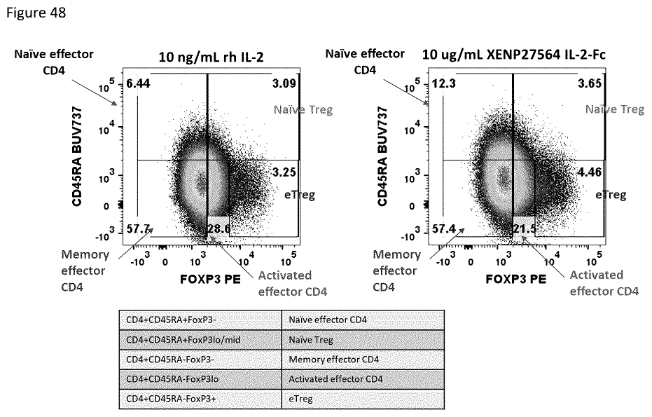

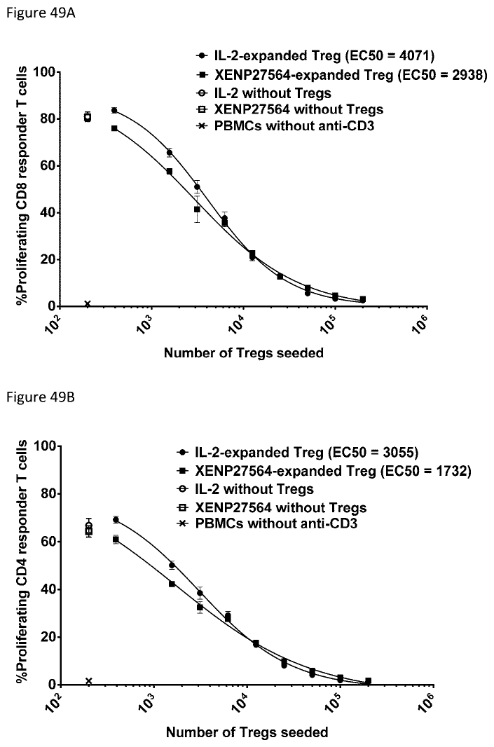

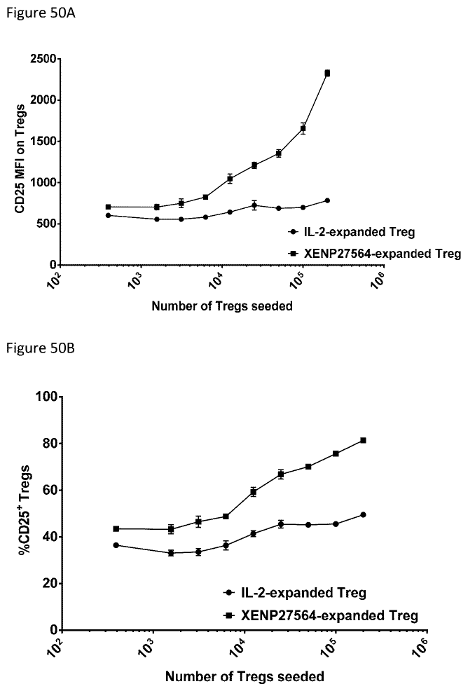

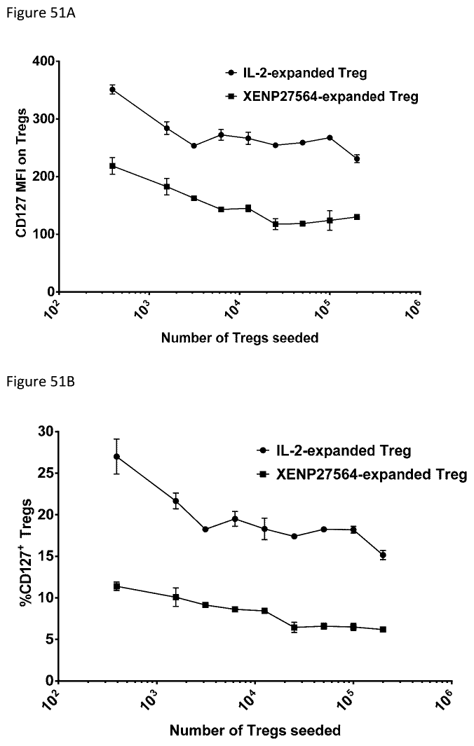

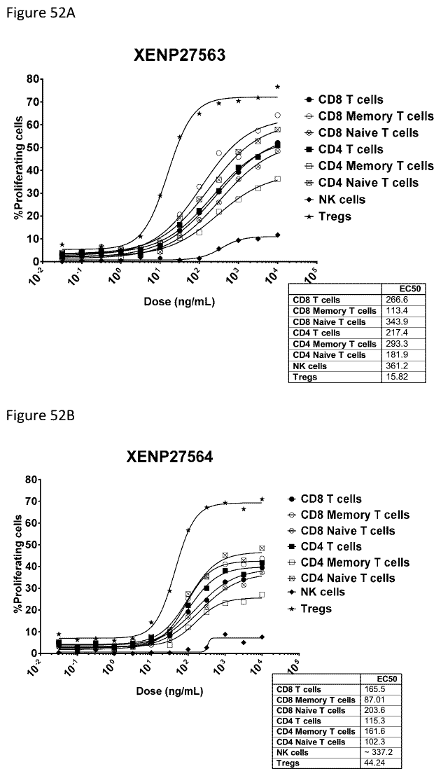

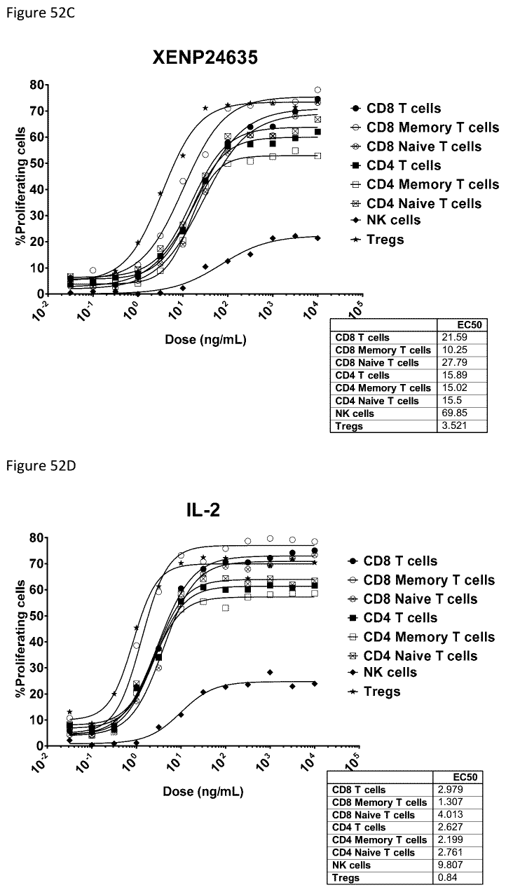

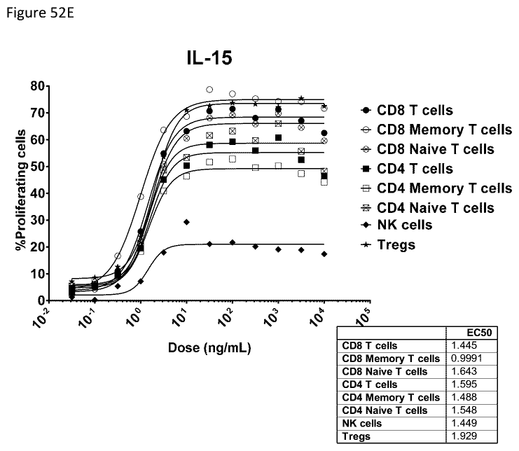

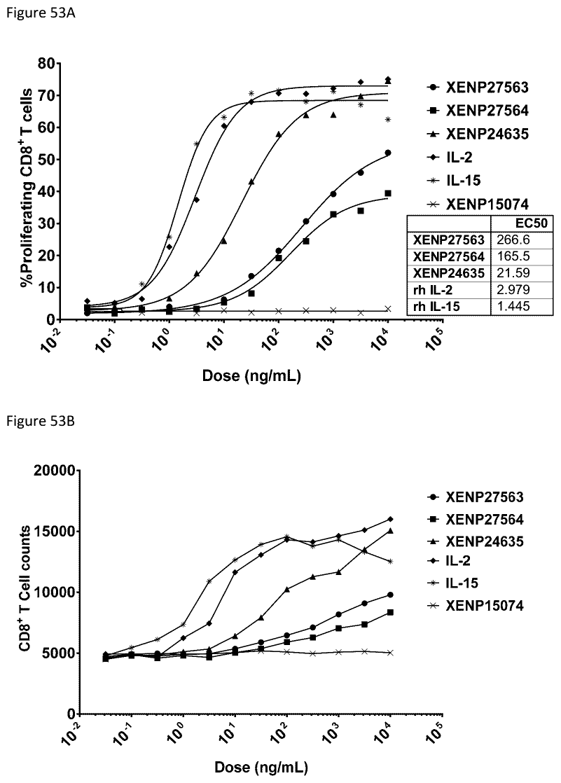

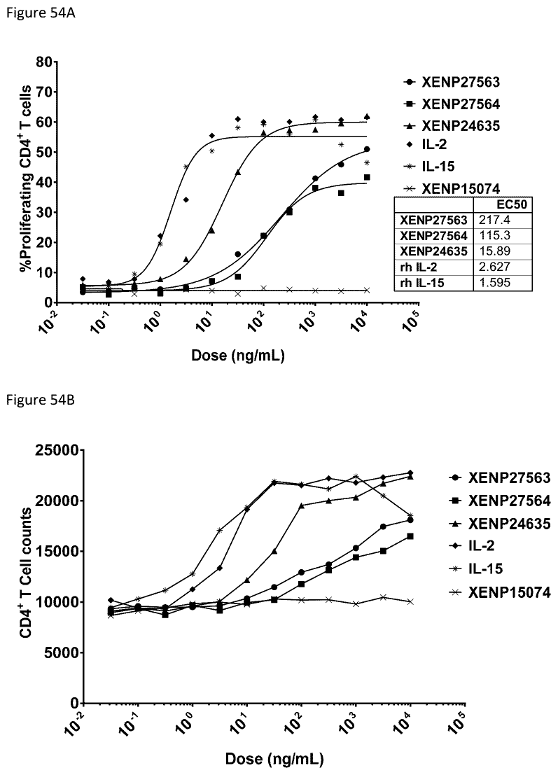

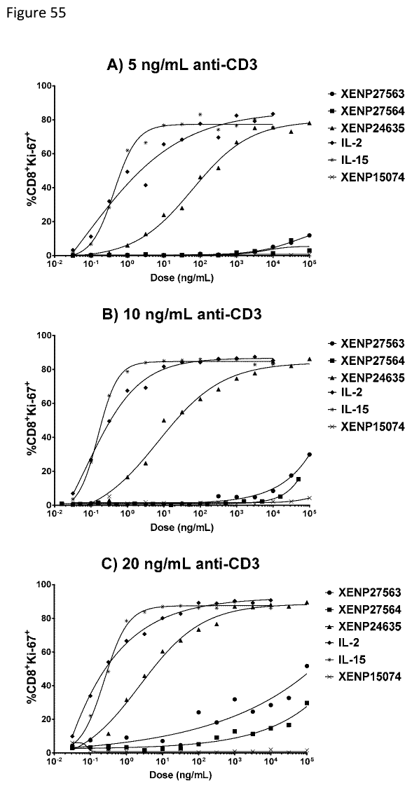

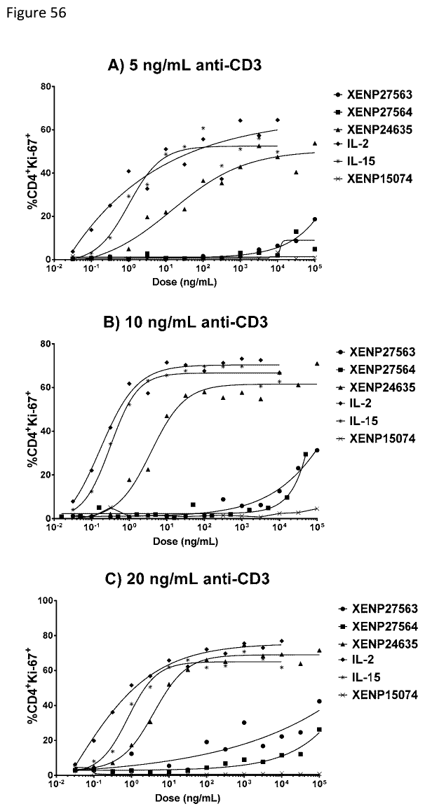

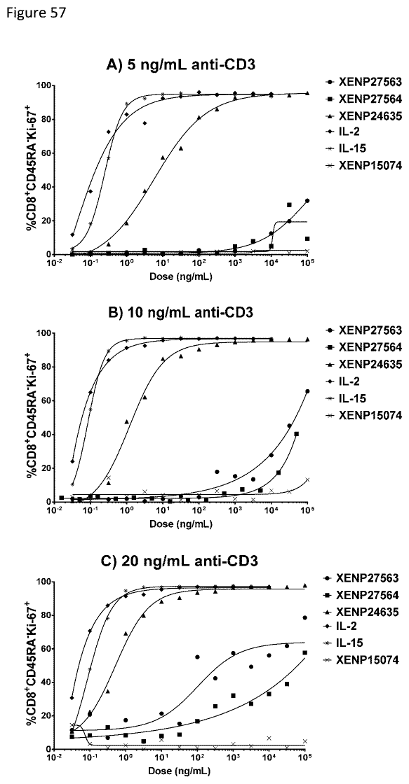

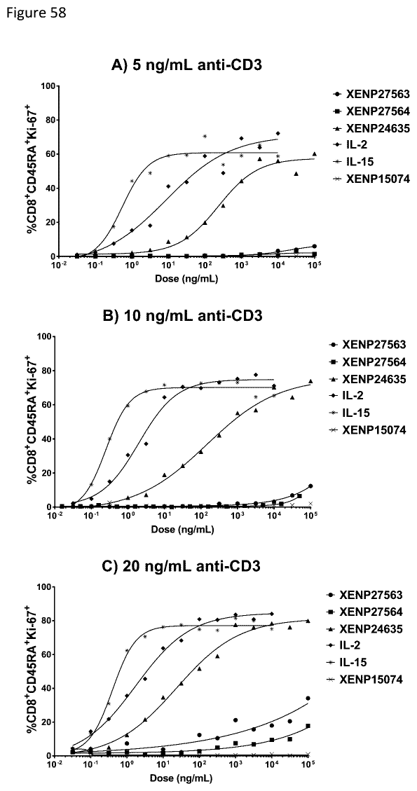

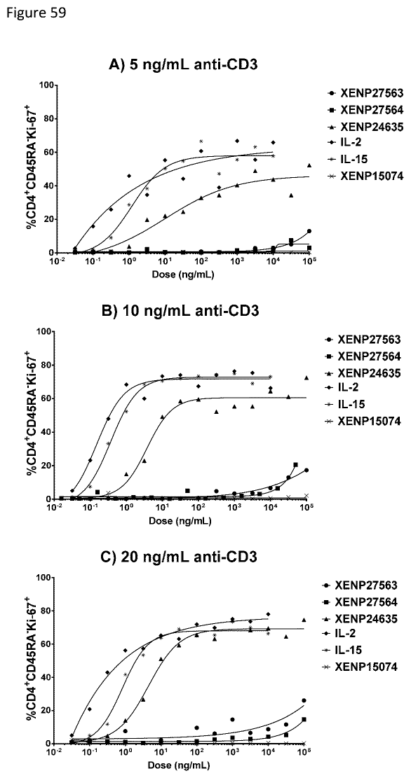

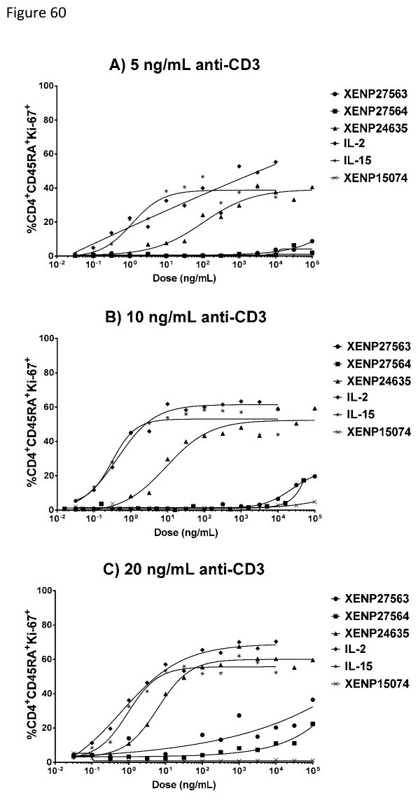

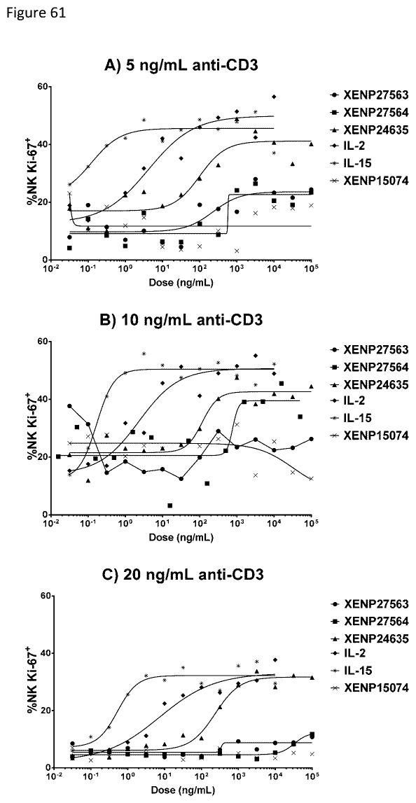

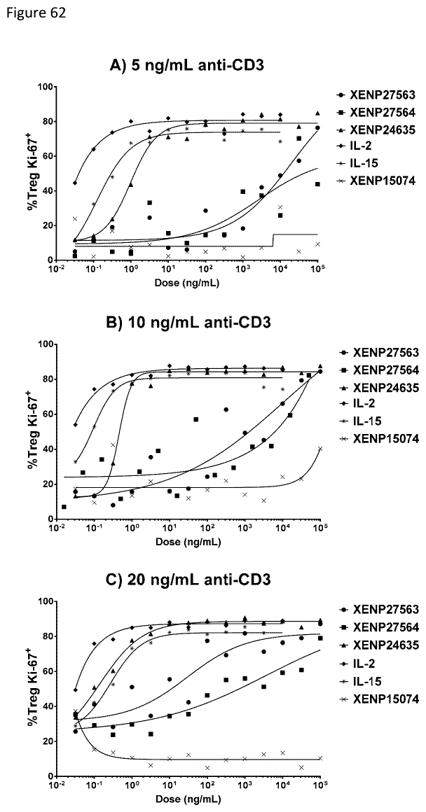

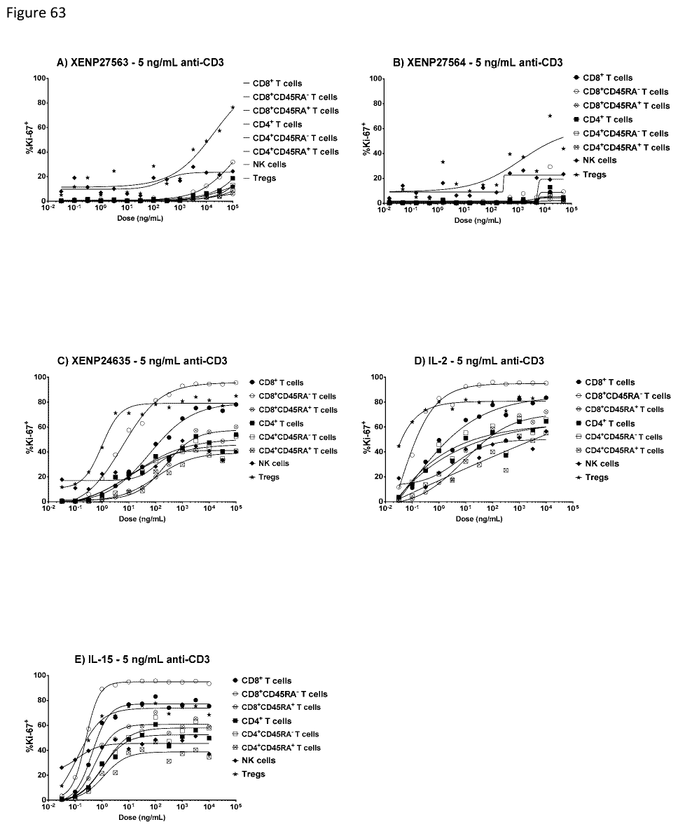

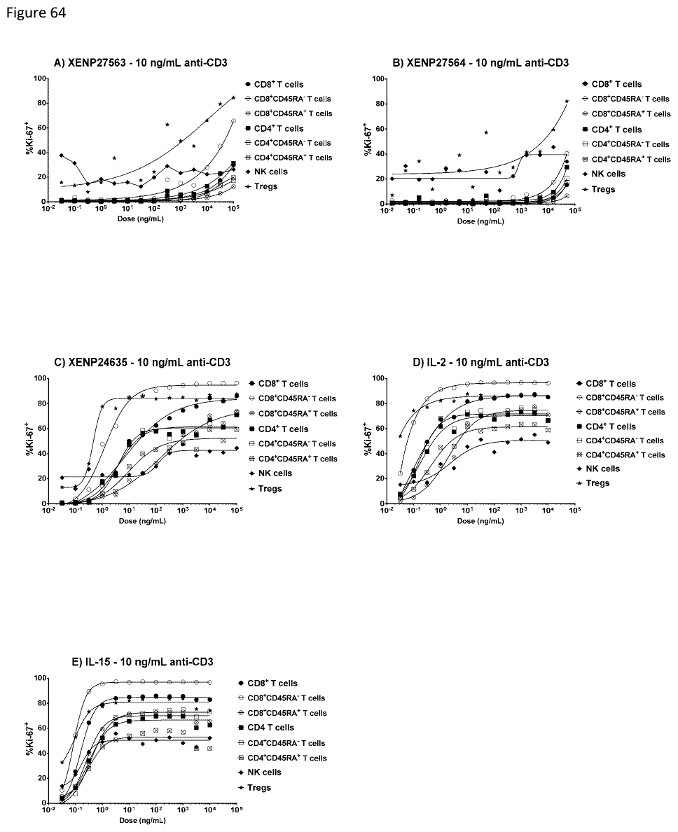

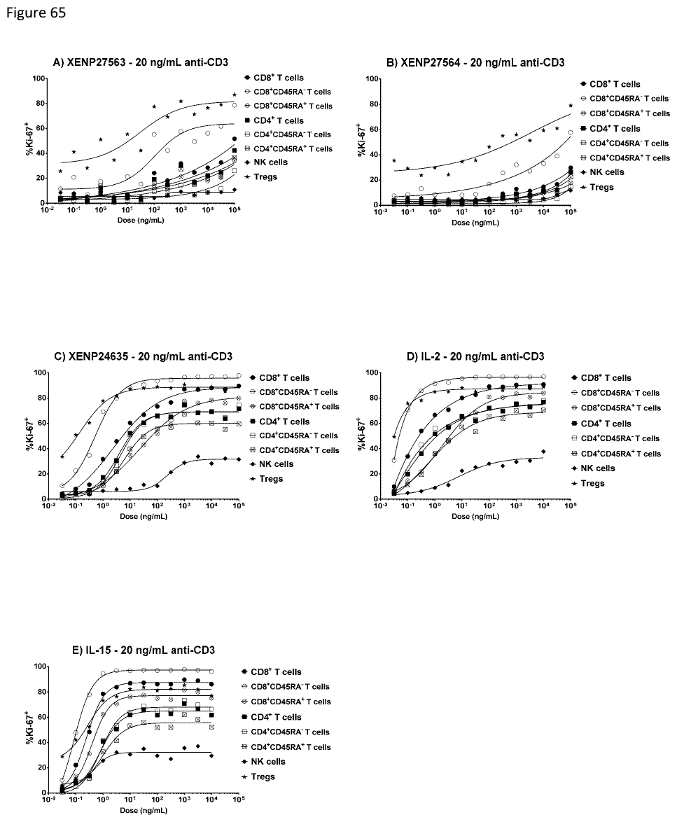

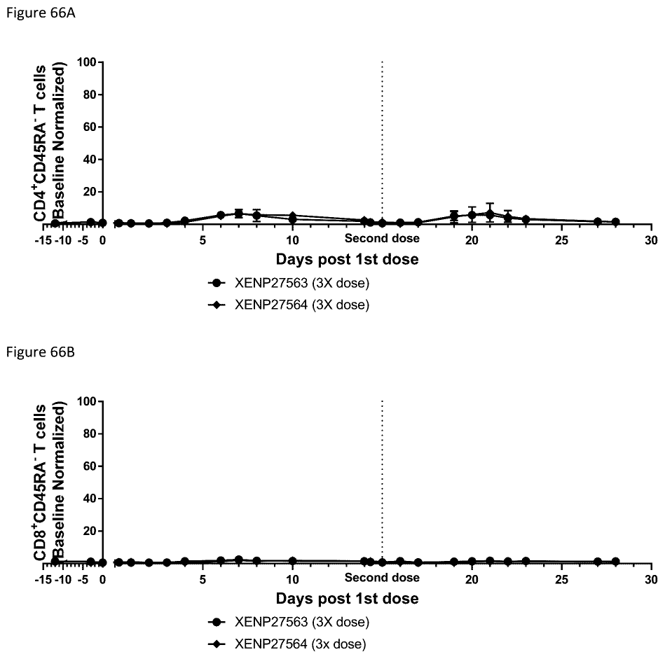

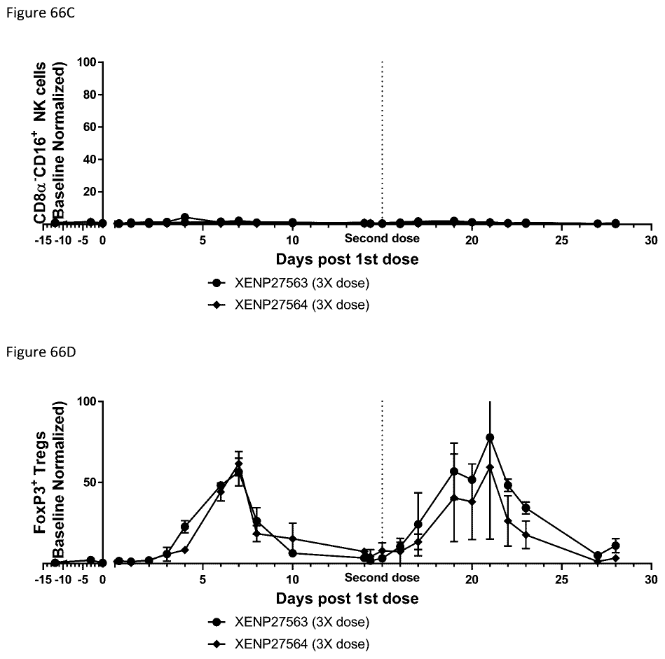

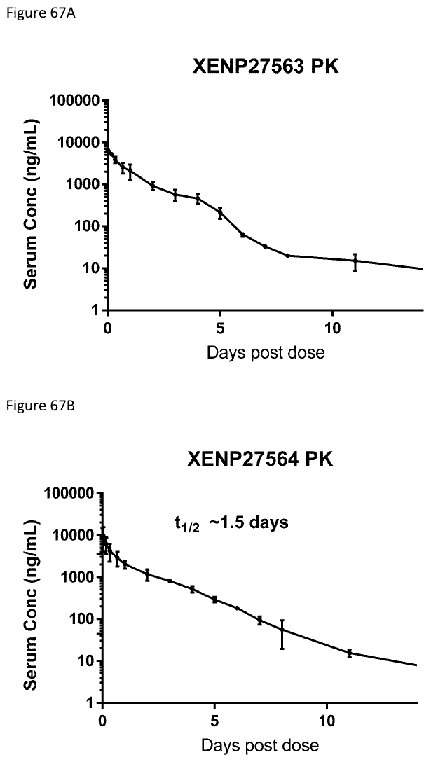

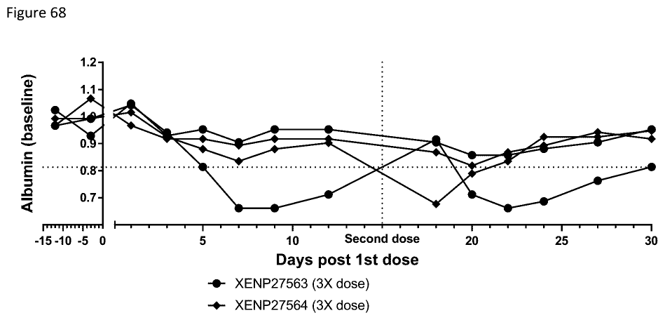

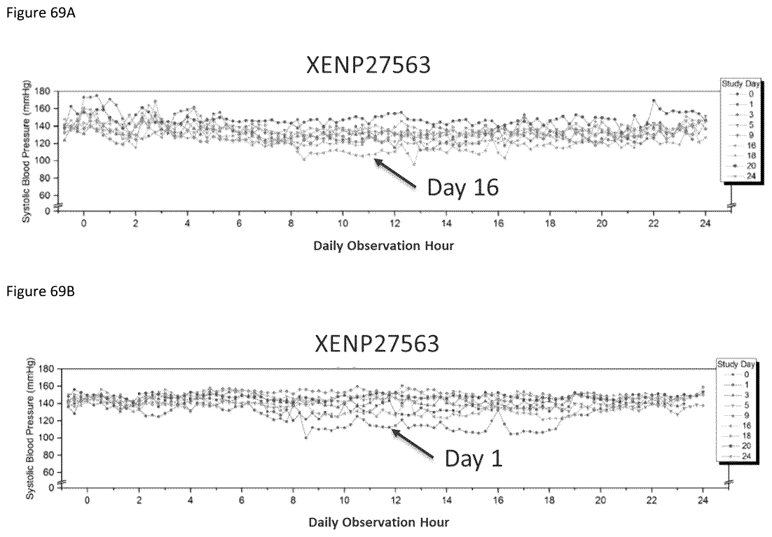

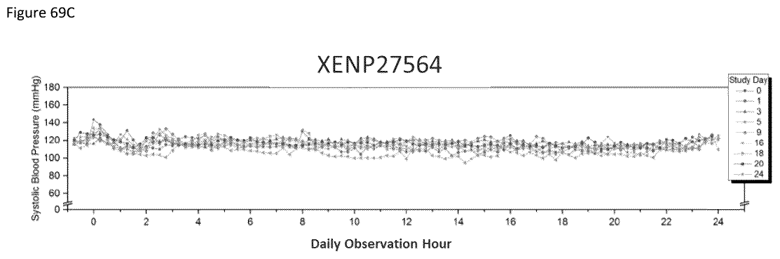

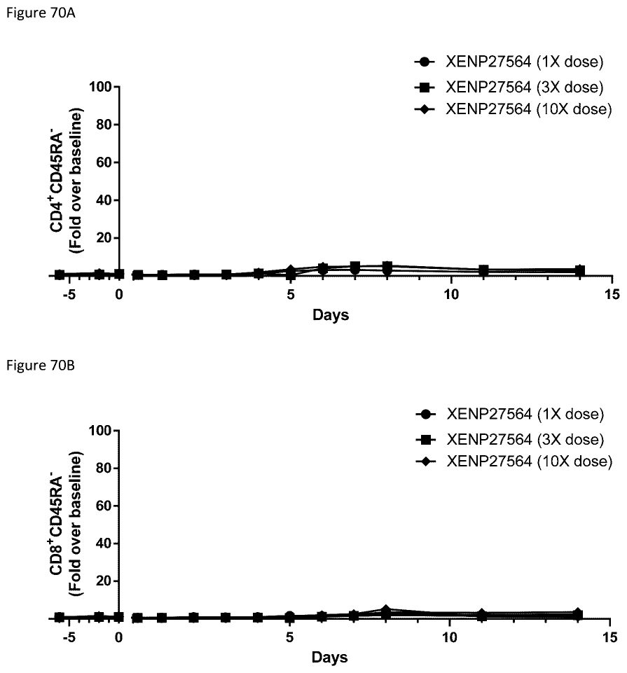

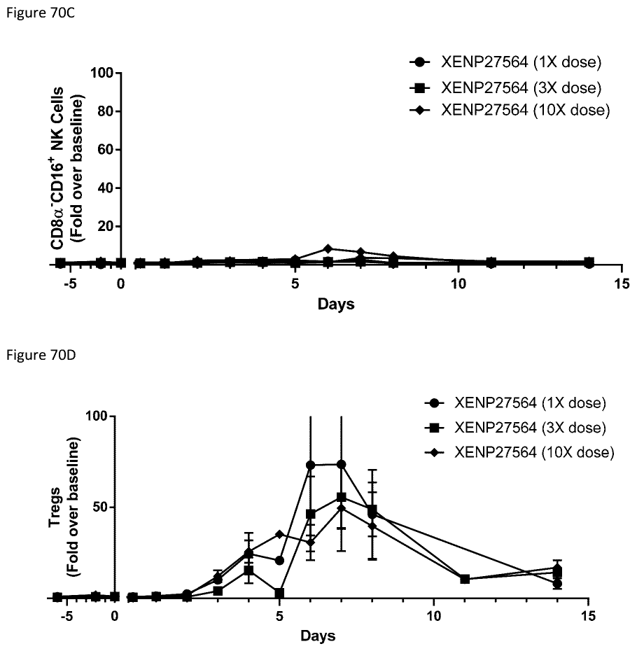

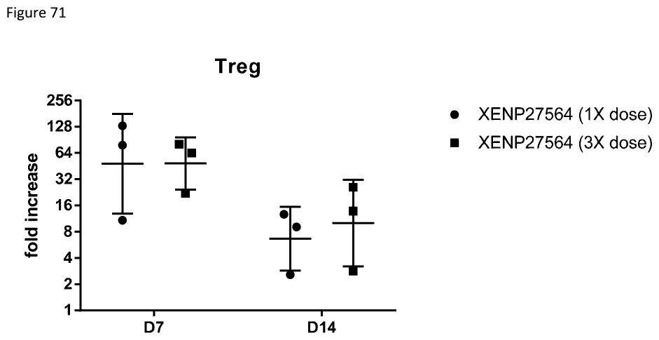

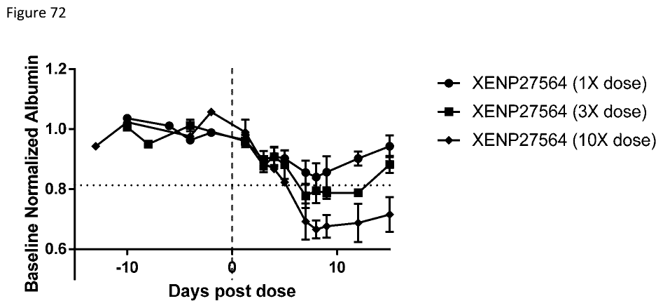

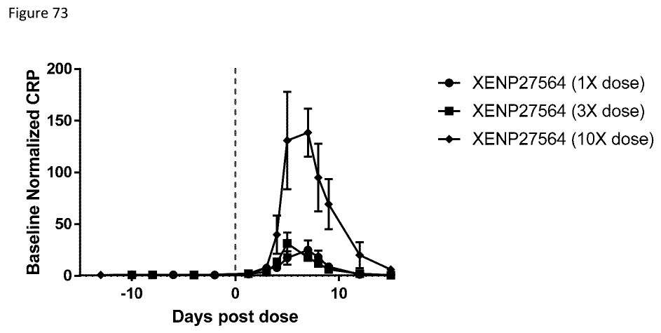

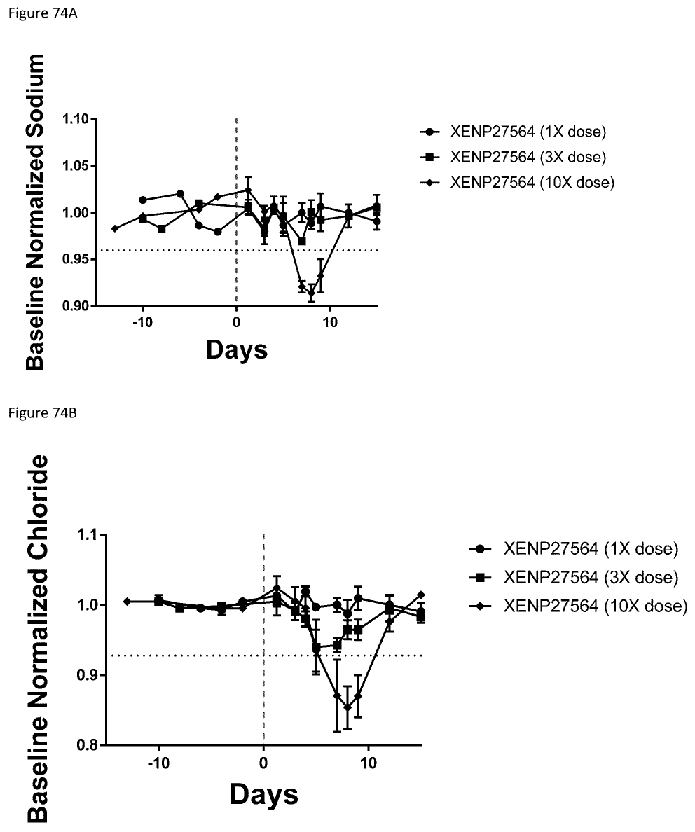

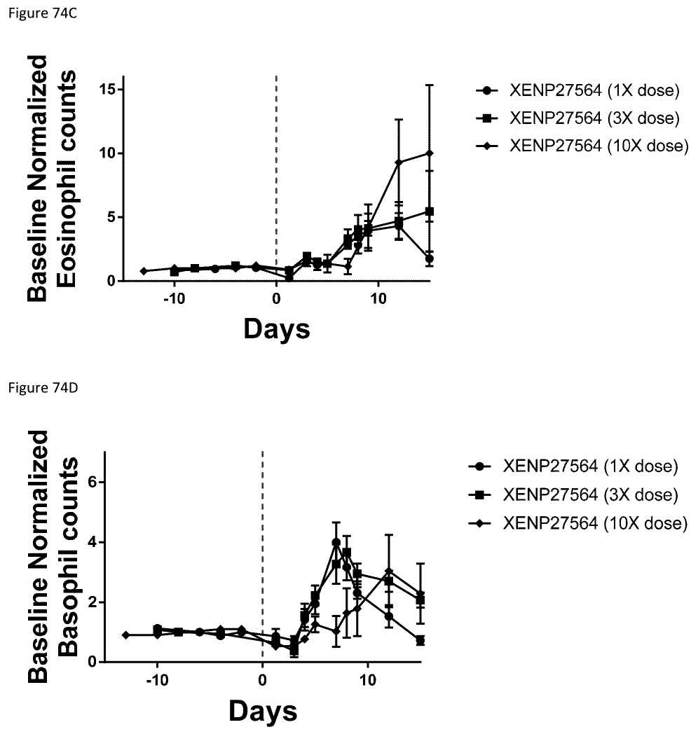

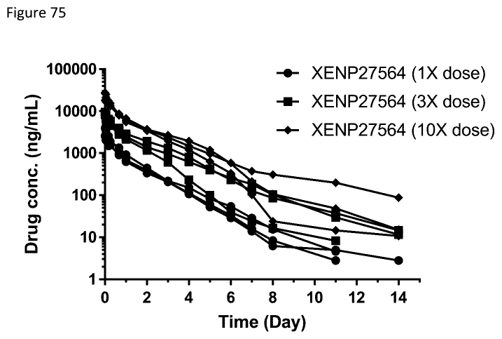

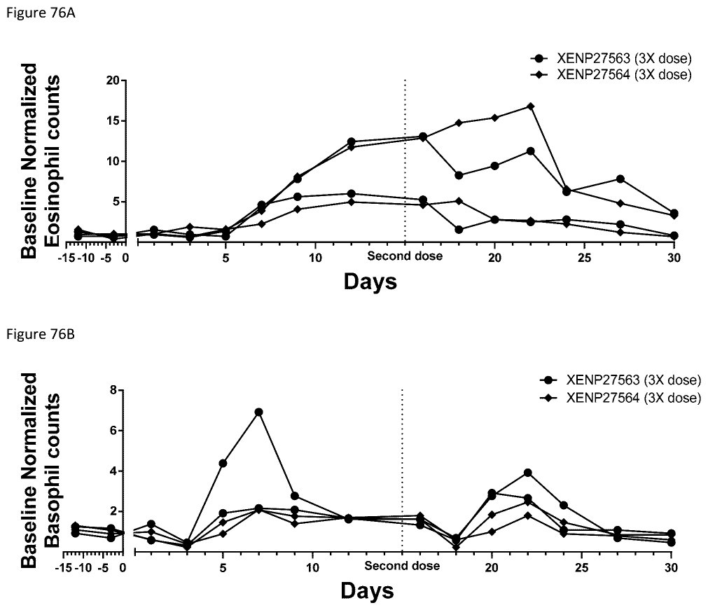

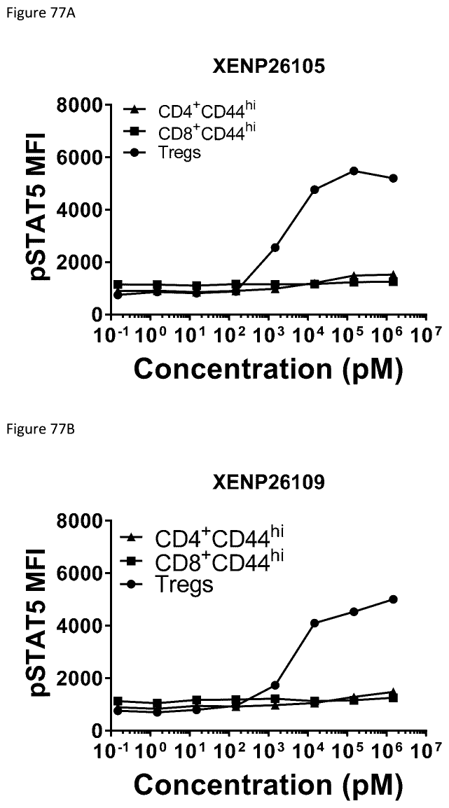

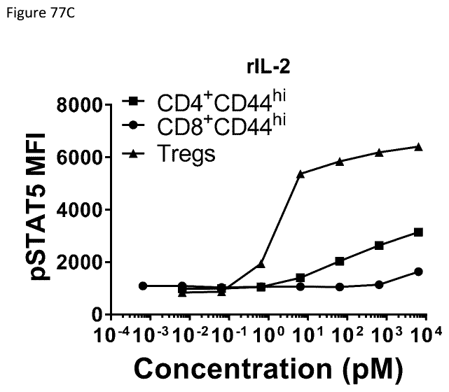

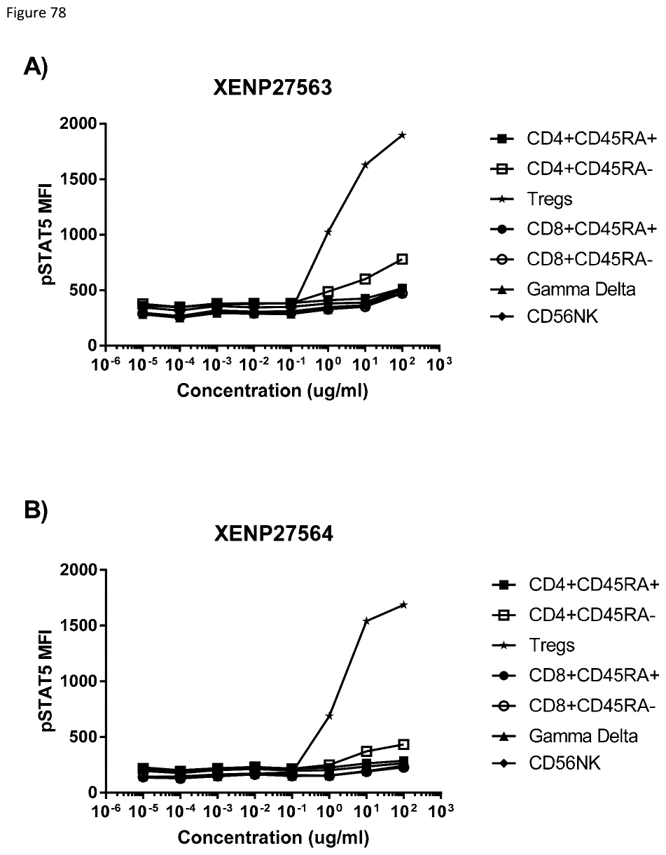

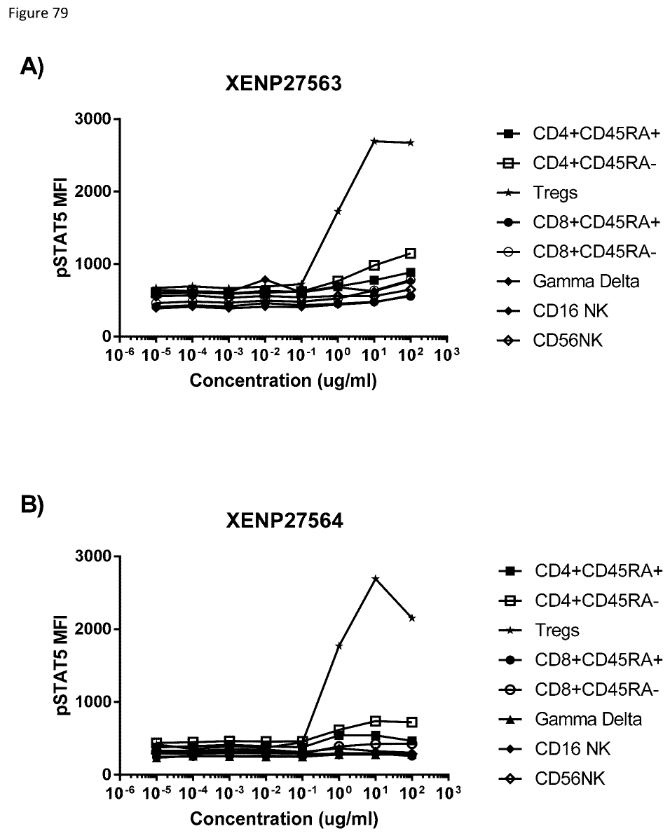

In one embodiment, “monomer 1” comprises the variant IL-2 domain comprising the amino acid substitutions T3A/D20N/N71K/C125S (as compared to wild type IL-2, SEQ IN NO:2), and an “monomer 1 Fc backbone” selected from those of FIGS. 6A-6D, including SEQ ID NOs:9, 11, 13, 15, 17, 19, 21, 23, 25, 27, 29, 31 or 33. In this embodiment, the “monomer 2” comprises an “empty Fc” selected from the “monomer 2 Fc backbones of SEQ ID NOs:10, 12, 14, 16, 18, 20, 22, 24, 26, 28, 30, 32 and 34.

In one embodiment, “monomer 1” comprises the variant IL-2 domain comprising the amino acid substitutions T3A/D20N/N71K/C125S (as compared to wild type IL-2, SEQ IN NO:2), and an “monomer 1 Fc backbone” selected from those of FIGS. 6A-6D, including SEQ ID NOs:9, 11, 13, 15, 17, 19, 21, 23, 25, 27, 29, 31 or 33, but with the FcRn variants M428L/N434S. In this embodiment, the “monomer 2” comprises an “empty Fc” selected from the “monomer 2 Fc backbones of SEQ ID NOs:10, 12, 14, 16, 18, 20, 22, 24, 26, 28, 30, 32 and 34, but with the FcRn variants M428L/N434S.

In one embodiment, “monomer 1” comprises the variant IL-2 domain comprising the amino acid substitutions T3A/D20N/T37R/C125S (as compared to wild type IL-2, SEQ IN NO:2), and a “monomer 1 Fc backbone” selected from those of FIGS. 6A-6D, including SEQ ID NOs:9, 11, 13, 15, 17, 19, 21, 23, 25, 27, 29, 31 or 33. In this embodiment, the “monomer 2” comprises an “empty Fc” selected from the “monomer 2 Fc backbones of SEQ ID NOs:10, 12, 14, 16, 18, 20, 22, 24, 26, 28, 30, 32 and 34.

In one embodiment, “monomer 1” comprises the variant IL-2 domain comprising the amino acid substitutions T3A/D20N/T37R/C125S (as compared to wild type IL-2, SEQ IN NO:2), and an “Fc backbone” selected from those of FIGS. 6A-6D, including SEQ ID NOs:9, 11, 13, 15, 17, 19, 21, 23, 25, 27, 29, 31 or 33, but with the FcRn variants M428L/N434S. In this embodiment, the “monomer 2” comprises an “empty Fc” selected from the “monomer 2 Fc backbones of SEQ ID NOs:10, 12, 14, 16, 18, 20, 22, 24, 26, 28, 30, 32 and 34 but with the FcRn variants M428L/N434S.

In these embodiments, preferred constructs include XENP26105, XENP27563, XENP26109 and XENP27564.

### B. Homodimeric Bivalent Constructs

In some embodiments, the I1-2-Fc fusion proteins of the invention are homodimeric bivalent constructs such as those depicted in FIGS. 19B and 19D. In this embodiment, the variant IL-2 domains are generally each fused to a variant human IgG1 Fc domain using either the hinge as the domain linker (generally including a C220S variant) or using an additional linker attached to the hinge.

In some embodiments, the variant IL-2 domains are attached a variant human IgG1 Fc domain (inclusive of the hinge with a C220S variant) and ablation variants E233P/L234V/L235A/G236del/S267K. In this embodiment, preferred constructs include a variant IL-2 domain with amino acid substitutions selected from the group consisting of T3A/D20N/T37R, T3A/D20N/T37R/C125S, T3A/D20N/T37R/C125A, T3A/D20N/N71K, T3A/D20N/N71K/C125S and T3A/D20N/N71K/C125A.

In some embodiments, the variant IL-2 domains are attached a variant human IgG1 Fc domain (inclusive of the hinge with a C220S variant), ablation variants E233P/L234V/L235A/G236del/S267K and FcRn variants M428L/N434S. In this embodiment, preferred constructs include a variant IL-2 domain with amino acid substitutions selected from the group consisting of T3A/D20N/T37R, T3A/D20N/T37R/C125S, T3A/D20N/T37R/C125A, T3A/D20N/N71K, T3A/D20N/N71K/C125S and T3A/D20N/N71K/C125A.

### IX. NUCLEIC ACIDS OF THE INVENTION

The invention further provides nucleic acid compositions encoding the homodimeric bivalent IL-2-Fc fusion proteins and the heterodimeric monovalent IL-2 fusion proteins.

As will be appreciated by those in the art, the nucleic acid compositions will depend on the format of the heterodimeric protein. Thus, for example, when the format requires two amino acid sequences, e.g. for the heterodimeric monovalent formats of FIG. 19A, two nucleic acid sequences can be incorporated into one or more expression vectors for expression.

Alternatively, when homodimeric bivalent formats as shown in FIG. 19B, a single nucleic construct and a single expression vector are used.

As is known in the art, the nucleic acids encoding the components of the invention can be incorporated into expression vectors as is known in the art, and depending on the host cells used to produce the heterodimeric Fc fusion proteins of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments.

In some embodiments, nucleic acids encoding each monomer, as applicable depending on the format, are each contained within a single expression vector, generally under different or the same promoter controls. In embodiments of particular use in the present invention, each of these two or three nucleic acids are contained on a different expression vector.

The heterodimeric Fc fusion protein of the invention are made by culturing host cells comprising the expression vector(s) as is well known in the art. Once produced, traditional fusion protein or antibody purification steps are done, including an ion exchange chromotography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the heterodimer (e.g., anionic exchange columns, cationic exchange columns). These substitutions also aid in the determination and monitoring of any contaminating homodimers post-purification (e.g., IEF gels, cIEF, and analytical IEX columns).

### X. FORMULATIONS

Formulations of the compositions used in accordance with the present invention are prepared for storage by mixing the fusion proteins having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions.

### XI. TREATMENT

The compositions of the IL-2-Fc fusion proteins of the invention find use in the treatment of autoimmune disease, by example by activating CD25+ cells of a patient using the dimeric proteins of the invention.

### XII. EXAMPLES

### A. Example 1: Engineering IL-2 for Increased Half-Life

As discussed above, IL-2 suffers from a very fast clearance. This rapid clearance is due in part to internalization of the IL-2:IL-2R complex. Subsequent to internalization into the endosome, IL-2, IL-2Rβ, and γc are degraded, while IL-2Rα is constitutively recycled to the cell-surface. IL-2 variant 2D1 which has L18M/L19S mutations has demonstrated a longer half-life than wild-type IL-2. Fallon et al. reported that wild-type IL-2 has a lower affinity for IL-2Rα at endosomal pH suggesting a pH dependent binding between IL-2 and IL-2Rα. The group further found that the extended half-life of 2D1 resulted from recycling with IL-2Rα due to 2D1 having a higher affinity for IL-2Rα at endosomal pH than WT IL-2 does. Notably, residues L18 and L19 on IL-2 have been described to contact IL-2RB and γc. In contrast, we hypothesized that modifying residues at the IL-2:IL-2Rα interface would be more suitable for attenuating pH dependent binding, increasing recycling of IL-2 with IL-2Rα, and by extension, increasing half-life.

### 1. Engineering IL-2 at the IL-2/IL-2Rα Interface to Attenuate pH Dependent Binding

By examining the crystal structure of the IL-2 and IL-2Rα interface (PDB code 2ERJ), we identified a possible natural “pH switch” at the IL-2/IL-2Rα interface consisting of the interaction between Arg38, Thr41, and/or Phe42 of IL-2 and His120 of IL-2Rα (see FIG. 8A-8B). We hypothesized that at low pH in the endosome after internalization of the IL-2:IL-2Rαβγ complex, His20 would become protonated, resulting in the release of IL-2 from IL-2Rα, and subsequent lysosomal degradation of the remaining IL-2:IL-2Rβγ complex. IL-2Rα is known to be recycled to the cell surface, and increasing the binding affinity of IL-2 with IL-2Rα at pH 6.0 by substituting one or more of these interacting residues could improve the recycling of IL-2 and prolong half-life. Using computational predictions by Protein Design Automation technology (see, e.g., WO 1998/047089, published Oct. 22, 1998), we generated variants to saturate these three contacting residues.

Plasmid coding for IL-2 was constructed by standard gene synthesis, followed by subcloning into a pTT5 expression vector. The IL-2 included a C-terminal polyhistidine tag (8×His) for purification and a C125S substitution for improved expression. Substitutions as predicted above were introduced by standard mutagenesis techniques. Proteins were produced by transient transfection in HEK293E cells and purified by Ni-NTA chromatography. Sequences for illustrative variants are depicted in FIGS. 9A-9H, with the polyhistidine tag removed.

Affinity of IL-2 for IL-2Rα at pH 7.4 and pH 6.0 were determined using Biacore, a surface plasmon resonance (SPR)-based technology. Experimental steps for Biacore generally included the following: Immobilization (capture of ligand onto a sensor chip); Association (flowing of various concentrations of analyte over the sensor chip); and Dissociation (flowing buffer over the sensor chips) in order to determine the affinity of the test articles. A reference flow with buffer alone was also included in the method for background correction during data processing. In this particular screen, human CD25 (IL-2Rα) was first captured on the sensor chips, and then various concentrations of the IL-2 variants were flowed over the sensor chips. Separate experiments were performed with the buffer at pH 7.4 to simulate conditions at the cell surface and with the buffer at pH 6.0 to simulate endosomal conditions. The resulting dissociation constants (K_{D}), association rates (kₐ), and dissociation rates (kₐ) are depicted in FIGS. 10A-10C and 11A-11C. Binding affinities and kinetic rate constants were obtained by analyzing the processed data using a 1:1 binding model. FIG. 12 depicts the fold improvement in dissociation rate at pH 6.0, while FIG. 13 depicts the fold improvement in dissociation rate at pH 7.4.

A large number of the variants with various substitutions at R38, T41, and F42 had worse or similar dissociation rates at pH 6.0 compared to WT IL-2; however, we identified some variants with drastically improved off-rates (that is, IL-2 variants which are more likely to be recycled with IL-2Rα). Favorite variants include XENP14142 (R38I) and XENP14144 (R38L). FIG. 14 depicts the Biacore sensorgrams for XENP14135 (wild-type IL-2 with C125S mutation) and XENP14142 (variant IL-2 with R38I and C125S). The similar dissociation curves for XENP14142 at pH 7.4 and pH 6.0 demonstrate successful attenuation of pH dependent binding.

2. Attenuating pH-Dependent Binding in the Context of Other IL-2 Variants

Next, R38L was combined with a prior art IL-2 variant (mutant 2-4 with Q126T as described in WO 2009/061853, published May 14, 2009) to investigate whether off-rates were improved in the context of other IL-2 variants.

As above, substitutions were introduced by standard mutagenesis techniques. Proteins were produced by transient transfection in HEK293E cells and purified by Ni-NTA chromatography. Sequences for the prior art variant described above with and without R38L are depicted in FIG. 15, with the polyhistidine tag removed.

Affinity of IL-2 for IL-2Rα at pH 7.4 and pH 6.0 were determined using Biacore as described in Example 1A. The resulting dissociation constants (K_{D}), association rates (kₐ), and dissociation rates (kₐ) as well as the ratio of affinity at pH 7.4 to affinity at pH6.0 are depicted in FIG. 16. The data shows that the ratio of affinities increased with the inclusion of the R38L substitution indicating successful attenuation of pH dependent binding.

### B. Example 2: Engineering IL-2 for Treg Selectivity

Proliferative signaling by IL-2 is mediated by IL-2RB and γc as part of the IL-2 intermediate-affinity receptor complex (IL-2Rβγ) or as part of the IL-2 high-affinity receptor complex (IL-2Rαβγ). CD25 confers the high affinity binding of the IL-2Rαβγ complex to IL-2 but is otherwise signaling deficient on its own. Due to the high affinity binding to the IL-2Rαβγ complex, IL-2 favors Tregs which constitutively express IL-2Rα. Therefore, it was hypothesized that increasing the affinity of IL-2 for IL-2Rα could further skew binding in favor of the IL-2Rαβγ complex on Tregs. Alternatively, decreasing the affinity of IL-2 for IL-2Rβ, γc, or IL-2Rβγ could skew binding away from the CD25-negative T cells and NK cells.

By examining the crystal structure of the interface between IL-2 and its receptors, as well as by modeling using MOE software, we predicted residues that may be substituted in order to increase IL-2 affinity for IL-2Rα or decrease IL-2 affinity for IL-2Rβ, γc, and/or IL-2Rβγ.

Plasmids coding for IL-2 were constructed by standard gene synthesis, followed by subcloning into a pTT5 expression vector. The IL-2 included a C-terminal polyhistidine tag (8×His) for purification and a C125S substitution for improved expression. Substitutions as predicted above were introduced by standard mutagenesis techniques. Proteins were produced by transient transfection in HEK293E cells and purified by Ni-NTA chromatography. Sequences for illustrative variants are depicted in FIGS. 17A-17D, with the polyhistidine tag removed.

Binding of IL-2 to its receptor components were determined using Octet, a BioLayer Interferometry (BLI)-based method. Experimental steps for Octet generally included the following: Immobilization (capture of ligand to a biosensor); Association (dipping of ligand-coated biosensors into wells containing serial dilutions of the analyte); and Dissociation (returning of biosensors to well containing buffer) in order to determine the affinity of the test articles. A reference well containing buffer alone was also included in the method for background correction during data processing. In particular, anti-human Fc (AHC) biosensors were used to capture either bivalent CD25(IL-2Rα)-Fc fusion, bivalent CD122(IL-2Rβ)-Fc fusion, or heterodimeric CD122:CD132(IL-2Rβγ)-Fc fusion and dipped in multiple concentrations of the IL-2 variants. The resulting BLI-response for the IL-2 variants were normalized against the BLI-response for XENP14135 (wild-type IL-2 with C125S) and depicted in FIGS. 18A-18B. Notably, several of the substitution at the IL-2:IL-2RB interface such as D20N and N88D drastically reduced or eliminated IL-2 binding to IL-2Rβ.

### C. Example 3: IL-2-Fc Fusion Proteins

In order to further address the short half-life of IL-2, we generated IL-2 as an Fc fusion (hereon referred to as IL-2-Fc fusions) with the goal of facilitating production and promoting FcRn-mediated recycling of the complex and prolonging half-life.

### 1. Production of IL-2-Fc Fusions

Plasmid coding for IL-2 was constructed by standard gene synthesis, followed by subcloning into a pTT5 expression vector containing Fc fusion partners (e.g., constant regions as depicted in FIGS. 6A-6D). The IL-2 may include C125S substitution for improved expression and T3A substitution to remove an O-glycosylation site. Cartoon schematics of illustrative IL-2-Fc fusion formats are depicted in FIGS. 19A-19D. Selected substitutions were introduced by standard mutagenesis techniques.

The monovalent IL-2-Fc or “monovIL-2-Fc” format (FIG. 19A) comprises IL-2 fused to the N-terminus of a first heterodimeric Fc-region (see for example, IL-2-Fc Backbone 1-monomer 2, in FIGS. 6A-6D), with the other side of the molecule being a “Fc-only” or “empty-Fc” heterodimeric Fc (see for example, IL-2-Fc Backbone 1-monomer 1, in FIGS. 6A-6D). Sequences for illustrative monovIL-2-Fc fusions are depicted in FIGS. 20A-20E. The bivalent IL-2-Fc or “bivIL-2-Fc” format (FIG. 19B) comprises IL-2 fused to the N-terminus of a homodimeric Fc-region (see for example, IL-2-Fc Backbone 12, in FIGS. 6A-6D). Sequences for illustrative bivIL-2-Fc fusions are depicted in FIG. 23. The monovIL-2-Fc fusions and bivIL-2-Fc fusions may have a variable length linker between the C-terminus of the IL-2 and the N-terminus of the Fc region (see FIG. 7 for non-limiting examples of domain linkers which may find use in the IL-2-Fc fusions, and FIGS. 19C-D for format pictures). Sequences for illustrative IL-2-Fc fusions with variable length linkers are depicted in FIG. 25A-25B.

Proteins were produced by transient transfection in HEK293E cells and were purified by a two-step purification process comprising protein A chromatography and anion exchange chromatography.

2. Investigation of Prior Art IL-2 Variants Engineered as monovIL-2-Fc Fusions

To investigate the robustness and efficacy of the monovIL-2-Fc fusion format, a number of prior art IL-2 variants were generated in this format. These IL-2-Fc fusions include XENP24637 (based on a variant described in WO 2012/107417, published Aug. 16, 2012), XENP24638 (based on mutant 2-4 as described in WO 2005/007121, published Jan. 27, 2005), XENP24639 (based on mutant M1 as described in WO 2005/007121, published Jan. 27, 2005), XENP24640 (based on mutant 2-4 with Q126T as described in WO 2009/061853, published May 14, 2009), XENP24642 (based on a variant described in WO 1999/060128, published Nov. 25, 1999), XENP24728 (based on H9-RET as described by Mitra et al. 2015), and XENP24729 (based on H9-RETR as described by Mitra et al. 2015). Additional variants, such as XENP24641, XENP24730, XENP24731, and XENP24732, were based on individual substitutions or combinations of substitutions described in the prior art. Sequences are depicted in FIGS. 20A-20E.

a. Affinity Screen of monovIL-2-Fc Fusions

Affinity of the above described monovIL-2-Fc fusions for the various IL-2 receptors were determined using Octet as generally described in Example 2. In particular to determine affinity for IL-2Rα, CD25(IL-2Rα)-Fc fusions (R&D Systems, Minneapolis, Minn.) were loaded onto AR2G biosensors and dipped into multiple concentrations of IL-2-Fc fusions. To determine affinity for IL-2RB and IL-2Rβγ, bivalent CD122(IL-2Rβ)-Fc-His fusions or heterodimeric CD122:CD132(IL-2Rβγ)-Fc-His fusion were loaded onto HIS1K biosensors and dipped into multiple concentrations of IL-2-Fc fusions. The resulting dissociation constants (K_{D}), association rates (kₐ), and dissociation rates (k_{d}) are depicted in FIG. 21.

b. Induction of STAT5 Phosphorylation by monovIL-2-Fc Fusions

Following binding of cytokines to their receptors, Janus kinases (JAKs) associated with the receptors phosphorylate STAT proteins which then translocate into the nucleus to regulate further downstream processes. Therefore, phosphorylation of STAT proteins (in particular, STAT5, which include STAT5a and STAT5b) is one of the earliest signaling events triggered by IL-2 binding to the high- or intermediate-affinity IL-2 receptors (Lin and Leonard (2000); Wuest et al. (2008)). Accordingly, the ability of the above described monovIL-2-Fc fusions to induce STAT5 phosphorylation in various cell types, such as CD8+ and CD4+ T cells and Tregs, was investigated.

Fresh PBMCs were incubated with the indicated IL-2-Fc test articles at the indicated concentrations for 15 minutes. Following incubation, PBMCs were stained with anti-CD3-BV510 (UCHT1), anti-CD4-BV605 (RPA-T4), and anti-CD8-Alexa700 (SKI) for 30-45 minutes at room temperature. Cells were washed and incubated with pre-chilled (−20° C.) 90% methanol for 20-60 minutes. After methanol incubation, cells were washed again and stained with anti-CD25-BV421 (M-A251), anti-CD45RA-PE (HI100), anti-FOXP3-Alexa488 (259D), and anti-pSTAT5-Alexa647 (pY687) to mark various cell populations and STAT5 phosphorylation. Data depicting induction of STAT5 phosphorylation on CD8⁺ T cells (CD3⁺CD8⁺CD25⁻), CD4⁺ T cells (CD3⁺CD4⁺CD25⁻), and Tregs (CD3⁺CD4⁺CD25⁺FOXP3⁺) are shown in FIGS. 22A-22F. Notably, IL-2-Fc fusions XENP24638 and XENP24642 were potent activators of Treg (as indicated by STAT5 phosphorylation) with minimal activation of CD8⁺ and CD4⁺ T cells, which is consistent with activity reported for the IL-2 variants which compose the IL-2-Fc fusions.

3. Investigation of Prior Art IL-2 Variants Engineered as bivIL-2-Fc Fusions

To investigate the robustness and efficacy of the bivIL-2-Fc fusion format, a prior art IL-2 variant (described in WO 1999/060128, published Nov. 25, 1999) as well as a control IL-2 (with C125S and T3A substitutions) were generated in this format, sequences for which are depicted in FIG. 23.

a. Induction of STAT5 Phosphorylation by bivIL-2-Fc Fusions

Fresh PBMCs were incubated with the indicated IL-2-Fc test articles at the indicated concentrations for 15 minutes. Following incubation, PBMCs were stained with anti-CD3-BUV395 (UCHT1), anti-CD4-BV605 (RPA-T4), and anti-CD8-Alexa700 (SKI) for 30-45 minutes at room temperature. Cells were washed and incubated with pre-chilled (−20° C.) 90% methanol for 20-60 minutes. After methanol incubation, cells were washed again and stained with anti-CD25-BV510 (M-A251), anti-CD45RA-PE (HI100), anti-FOXP3-Alexa488 (259D), and anti-pSTAT5-Alexa647 (pY694) to mark various cell populations and STAT5 phosphorylation. Data depicting induction of STAT5 phosphorylation on CD8⁺ T cells, CD4⁺ T cells, and Tregs are shown in FIG. 24A-24B.

4. Investigation of Prior Art IL-2 Variants Engineered as IL-2-Fc Fusions with Domain Linkers

To investigate the effect of including linkers between the IL-2 and the Fc region, prior art IL-2 variants (as described in WO 1999/060128, published Nov. 25, 1999, and in WO 2012/107417, published Aug. 16, 2012) were generated as monovIL-2-Fc fusions or bivIL-2-Fc fusions with Gly-Ser linkers. Sequences for these IL-2-Fc fusions are depicted in FIG. 25A-25B.

a. Induction of STAT5 Phosphorylation by IL-2-Fc Fusions with Domain Linkers

Fresh PBMCs were incubated with the indicated IL-2-Fc test articles at the indicated concentrations for 15 minutes. Following incubation, PBMCs were stained with anti-CD3-BUV395 (UCHT1), anti-CD4-BV605 (RPA-T4), and anti-CD8-Alexa700 (SKI) for 30-45 minutes at room temperature. Cells were washed and incubated with pre-chilled (−20° C.) 90% methanol for 20-60 minutes. After methanol incubation, cells were washed again and stained with anti-CD25-BV510 (M-A251), anti-CD45RA-PE (HI100), anti-FOXP3-Alexa488 (259D), and anti-pSTAT5-Alexa647 (pY694) to mark various cell populations and STAT5 phosphorylation. Data depicting induction of STAT5 phosphorylation on CD8⁺ T cells, CD4⁺ T cells, and Tregs are shown in FIG. 26A-26E.

### D. Example 4: Engineering Variant IL-2-Fc Fusions with Increased Affinity for CD25 and Decreased Affinity for CD122

As discussed in Example 2, increasing the affinity of IL-2 for IL-2Rα could further skew binding in favor of the IL-2Rαβγ complex on Tregs, while decreasing the affinity of IL-2 for IL-2RB, γc, or IL-2Rβγ could skew binding away from the CD25-negative T cells and NK cells. Here, substitutions which increased IL-2Rα binding were combined with substitutions which decreased IL-2RB binding in the context of IL-2-Fc fusions with the aim to enhance Treg selectivity.

Plasmid coding for IL-2 was constructed by standard gene synthesis, followed by subcloning into a pTT5 expression vector containing Fc fusion partners (e.g., constant regions as depicted in FIGS. 6A-6D). The IL-2 included a C125S substitution for improved expression and T3A substitution to remove an O-glycosylation site. Selected substitutions as described in Example 2 were introduced by standard mutagenesis techniques. Proteins were produced by transient transfection in HEK293E cells and were purified by a two-step purification process comprising protein A chromatography and anion exchange chromatography. Sequences for illustrative IL-2-Fc fusions engineered for increased CD25 affinity and/or decreased CD122 affinity are depicted in FIG. 27A-27E.

1. Affinity Screen of Variant IL-2-Fc Fusions Engineered for Increased CD25 affinity and decreased CD122 affinity

Affinity of the above described variant IL-2-Fc fusions for the various IL-2 receptors were determined using Octet as generally described in Example 3B(a). In particular to determine affinity for IL-2Rα, CD25(IL-2Rα)-Fc fusions (R&D Systems, Minneapolis, Minn.) were loaded onto AR2G biosensors and dipped into multiple concentrations of IL-2-Fc fusions. To determine affinity for IL-2RB and IL-2Rβγ, bivalent CD122(IL-2Rβ)-Fc-His fusions or heterodimeric CD122:CD132(IL-2Rβγ)-Fc-His fusion were loaded onto HISIK biosensors and dipped into multiple concentrations of IL-2-Fc fusions. The resulting dissociation constants (K_{D}), association rates (kₐ), and dissociation rates (k_{d}) are depicted in FIG. 33.

### 2. STAT5 Phosphorylation by Variant IL-2-Fc Fusions Engineered for Increased CD25 Affinity and Decreased CD122 Affinity on Various Cell Populations

Fresh PBMCs were incubated with the indicated IL-2-Fc test articles at the indicated concentrations for 15 minutes. Following incubation, PBMCs were stained with anti-CD3-BV396 (UCHT1), anti-CD4-BV605 (RPA-T4), and anti-CD8-Alexa700 (SKI) for 30-45 minutes at room temperature. Cells were washed and incubated with pre-chilled (−20° C.) 90% methanol for 20-60 minutes. After methanol incubation, cells were washed again and stained with anti-CD25-BV421 (M-A251), anti-CD45RA-PE (HI100), anti-FOXP3-Alexa488 (259D), and anti-pSTAT5-Alexa647 (pY687) to mark various cell populations and STAT5 phosphorylation. Data depicting induction of STAT5 phosphorylation on various cell populations are depicted in FIGS. 28A-29B.

The data show that in comparison to controls XENP24635 (IL-2-Fc with C125S only) and recombinant human IL-2 (rhIL-2), many of the illustrative variants were potent activators of Tregs with minimal induction of STAT5 phosphorylation on CD25-negative T cells (CD8+ and CD4+).

### E. Example 5: Engineering Additional Variant IL-2-Fc Fusions

Additional IL-2-Fc fusions were engineered combining characteristics as described in the preceding examples including valency, domain linkers, pH switch, and Treg selectivity, and produced as generally described in Example 2. Illustrative sequences are depicted in FIG. 30A-30E. Additionally, an Fc-IL-2(V91K/C125A) fusion engineered to increase the ratio of Tregs to non-regulatory T cells as described in WO 2014/153111 was generated as a comparator (herein referred to as XENP27193; sequence depicted in FIG. 34).

### 1. Induction of STAT5 Phosphorylation by Additional Monovalent IL-2-Fc Fusions

Fresh PBMCs were incubated with the indicated IL-2-Fc test articles at the indicated concentrations for 15 minutes at 37° C. Following incubation, PBMCs were first stained with anti-CD3-BUV395 (UCHT1), anti-CD4-BV605 (RPA-T4), anti-CD8-AF700 (SKI), and anti-CD56-PE antibodies. Following the first stain, cells were permeabilized using PerFix EXPOSE (Beckman Coulter, Indianapolis, Ind.). Following permeabilization, cells were stained with anti-CD25-BV421 (M-A251), anti-CD45RA-BV510 (HI100), anti-FoxP3-AF488 (259D), and anti-pSTAT5-AF647 (47/Stat5(pY694)) antibodies. Following the second staining, the cells were analyzed by flow cytometry to investigate STAT5 phosphorylation on various lymphocyte populations. Data depicting pSTAT5 MFI on various lymphocyte populations, indicating signaling by the IL-2-Fc fusions via IL-2 receptors, are depicted in FIG. 35A-35E.

The data show that each of the variants induced STAT5 phosphorylation on Tregs. Notably, the variant IL-2-Fc fusions preferentially induced Tregs over CD4⁺ memory T cells (CD45RA⁻), CD8⁺ memory T cells (CD45RA⁻), NK cells, and γδ T cells in comparison to both recombinant IL-2 and WT monovalent IL-2-Fc fusion (XENP24635). In comparison, prior art variant Fc-IL-2 fusion XENP27193 was comparatively less Treg selective.

2. Bivalent IL-2-Fc Fusions are More Potent than Monovalent Counterparts

Induction of STAT5 phosphorylation by the various IL-2-Fc test articles were investigated as described in Example 5A. Data depicting pSTAT5 MFI on Tregs and CD4⁺ memory T cells (CD45RA⁻), indicating signaling by the IL-2-Fc fusions via IL-2 receptors, are depicted in FIGS. 36-42.

The data show that for each IL-2 variant, bivalent IL-2-Fc versions were more potent in inducing STAT5 phosphorylation on Tregs than corresponding monovalent IL-2-Fc versions. Notably, engineering domain linkers between the IL-2 component and the Fc component (for example in XENP27002, XENP27003, XENP27004, XENP27005, XENP27006, and XENP27007) further enhances the potency of the bivalent IL-2-Fc fusions. Notably, each of the bivalent IL-2-Fc fusion constructs (with and without linkers) retained selectivity for Tregs over other lymphocyte populations such as CD4⁺CD45RA⁻ T cells.

### F. Example 6: Maximizing Exposure of IL-2-Fc Fusions

### 1. Incorporating Xtend Fe

IL-2-Fc fusions as described above were engineered with Xtend Fc (M428L/N434S) with enhanced binding to FcRn to further promote FcRn-mediated recycling of the fusions and subsequently prolonging circulating half-life. Sequences for illustrative bivalent IL-2-Fc fusions with Xtend Fc are depicted in FIGS. 43A-43B, and sequences for illustrative monovalent IL-2-Fc fusions with Xtend Fc are depicted in FIGS. 44A-44D.

2. Selecting IL-2-Fc Fusions with a Balance of Treg Selectivity and Potency

We also reasoned that an IL-2-Fc fusion having a lower potency would decrease antigen sink, and thus, increase the circulating half-life. In view of data in Example 5B demonstrating that bivalent IL-2-Fc fusions and IL-2-Fc fusions having domain linkers had enhanced potency, we were particularly interested in monovalent IL-2-Fc fusions lacking domain linkers. Accordingly in order to identify IL-2-Fc fusions with optimal balance between selectivity and potency, we compared the in vitro potency (as indicated by induction of STAT5 phosphorylation on various lymphocyte populations) of monovalent IL-2 fusions (XENP26105 and XENP26109, respectively comprising D20N/T37R and D20N/N71K variants) with XENP24635 (monovalent IL-2-Fc fusion with C125S mutation), as well as XENP25908 and XENP27193 (which are comparator IL-2-Fc fusions indicated for treatment of autoimmune diseases), in a STAT5 phosphorylation assay, data for which are depicted in FIG. 45A-45E.

Notably, both XENP26105 and XENP26109 were less potent than XENP24635 and XENP27193 (respectively EC50s of 1 nM and 5 nM vs. 0.02 nM and 0.01 nM), but were able to achieve similar levels of activity on Tregs at higher doses while maintaining selectivity for Tregs. While XENP26105 had a potency comparable to XENP25908 (respectively EC50s of 1 nM vs 0.7 nM), the data show that XENP26105, as well as XENP26109, were able to achieve much higher levels of activity on Tregs than XENP25908. The reduced potency and selectivity observed for XENP26105 and XENP26109 suggests that they would be useful for selective and sustained Treg expansion in a clinical setting. Accordingly, we further investigated the potential of XENP27563 and XENP27564, the Xtend Fc analogs of XENP26105 and XENP26109.

### G. Example 7: In Vitro Characterization of XENP27563 and XENP27564

1. Treg Cultures Treated with CD25-Selective IL-2-Fc Fusions Show Greater CD25 Expression

It has been previously reported that rapamycin promotes proliferation of CD4+CD25+FOXP3+ T regs in vitro, and resulting expanded Tregs suppress CD4+ and CD8+ T cell proliferation (see, for Example, Battaglia et al. (2006) Rapamycin promotes expansion of functional CD4⁺CD25+FOXP3+ regulatory T cells of both healthy subjects and type 1 diabetic patients. J Immunol. 177(12) 8338-8347; and Strauss et al. (2007) Selective survival of naturally occurring human CD4⁺CD25+Foxp3+ regulatory T cells cultured with rapamycin. J Immunol. 178(1) 320-329).

CD4+ T cells were enriched from human PBMCs by negative selection using EasySep™ Human CD4+ T Cell Enrichment Kit (STEMCELL Technologies, Vancouver, Canada). Treg were expanded using Dynabeads™ Human Treg Expander (Thermo Fisher Scientific, Waltham, Mass.) in RPMI1640+10% fetal bovine serum+0.1 μg/ml rapamycin+500 U/ml IL-2 for 1-4 days. Tregs were transferred to T75 flasks coated with 0.5 μg/ml anti-CD3 (OKT3, Biolegend, San Diego, Calif.) and cultured with RPMI1640+10% fetal bovine serum+0.1 μg/ml rapamycin+100 U/ml IL-2+0.5 μg/ml anti-CD28 mAb. Experiments were performed at least 8 days after initial enrichment of CD4+ T cells from PBMCs. Tregs enriched and cultured as such are hereon referred to as rapamycin Treg.

Rapamycin Tregs were further cultured with 0.5 μg/ml plate-bound anti-CD3 (OKT3) in RPMI1640 media containing 10% FBS, 0.5 μg/ml anti-CD28 mAb, 100 ng/ml rapamycin, and either 10 ng/ml recombinant IL-2 or 10 μg/ml of XENP27564 (IL-2-Fc fusion having IL-2(D20N/N71K/C125S) variant). 14 days after culture, Tregs were stained with anti-CD25-FITC (M-A251), anti-FoxP3-PE (PCH101), anti-CTLA-4-PE-Dazzle594 (L3D10), anti-PD-1-BB700 (EH12.1), anti-GITR-PE-Cγ7 (108-17), anti-Ki67-Alexa647, anti-ICOS-Alexa700 (C398.4a), anti-TIGIT-BV421 (A15153G), anti-LAG-3 (11C3C65), anti-CCR4-BV605 (L291H4), anti-CD8-BV650 (SKI), anti-CD39-BV711 (A1), anti-TIM-3-BV785 (F38-2E2), anti-CD40BUV396 (SK3), anti-CD3-BUV496 (UCHT1), anti-CD45-BUV805 (HI30), anti-CD45RA-BUV737 (HI100), and Zombie NIR (APC-Cγ7) and analyzed using flow cytometry, data for which are depicted in FIGS. 46-47. The data show that Treg treated with CD25-selective XENP27564 show greater CD25 expression. Further as depicted in FIG. 48, XENP27564 showed greater expansion of effector Treg (CD45RA-FoxP3^{mid-high}) population.

2. 7B: Treg Cultures Treated with CD25-Selective IL-2-Fc Fusions Show Greater Suppressive Function

The rapamycin Treg further cultured with either IL-2 or XENP27564 as described in Example 7A were assessed for their suppressive function on Day 15. 1×105 CFSE-labeled PBMCs were incubated with indicated number of Tag-it Violet labeled Tregs for 4 days, and expansion of CD8+ responder and CD4+ responder was determined by CFSE-dilution. Lymphocyte populations were stained as follows: anti-CD8-PerCp-By5.5 (SKI), anti-CD3-PE-Cγ7 (OKT3), anti-CD127-APC (A019D5), anti-CD25-APC-Fire750 (M-A251), anti-CD45RO-Alexa700 (UCHL1), anti-CD16-BV605 (3G6), anti-CD56-BV605 (HCD56), anti-CD45RA-BV785 (HI100), anti-CD4-BUV395 (SK3), and Zombie Aqua (BV510). Notably, the data as depicted in FIG. 49A-49B indicate that Treg expanded by Treg-selective IL-2-Fc fusions may have enhanced suppressive function.

Additionally, we investigated the expression of CD25 and CD127 on Tregs in the suppression assay, data for which are depicted in FIGS. 50A-51B. Consistent with the data above, the XENP27564-expanded Tregs showed greater levels of CD25 expression. Notably, the XENP27564-expanded Tregs showed lower expression of CD127, a marker which has been previously found to inversely correlate with the suppressive function of Tregs (Liu et al. (2006) CD127 expression inversely correlates with FoxP3 and suppressive function of human CD4+ T reg cells. J Exp Med. 203(7): 1701-1711). This may explain the enhanced suppressive function observed for XENP27564-expanded Tregs.

3. CD25-Selective IL-2-Fc Fusions Show Treg Selectivity and Suppressive Effect on Other Lymphocyte Populations after Activation with Anti-CD3

In a suppression assay, CFSE-labeled PBMCs and Tag-it Violet labeled rapamycin Tregs were incubated with indicated concentrations of indicated test articles for 4 days with 100 ng/ml plate-bound anti-CD3 (OKT3). Lymphocyte populations were stained as follows: anti-CD8-PerCp-Cγ5.5 (SKI), anti-CD3-PE-Cγ7 (OKT3), anti-CD25-APC-Cγ7 (M-A251), anti-CD45RO-Alexa700 (UCHL1), anti-CD16-BV605 (3G6), anti-CD56-BV605 (HCD56), anti-CD45RA-BV785 (HI100), anti-CD4-BUV395 (SK3), and Zombie Aqua (BV510). Proliferation of various lymphocyte populations (as determined by CFSE or Tag-it Violet dilution; Zombie dγc used to exclude dead cells) following treatment with the test articles are depicted in FIGS. 52A-52E. The data show that CD25− selective IL-2-Fc fusions XENP27563 and XENP27564 selectively expanded Tregs in comparison to XENP24635 (IL-2-Fc with C125S only), recombinant IL-2, and recombinant IL-15. In fact, the data depicted in FIGS. 53A and 54B show that the CD25-selective IL-2-Fc fusions had substantially reduced potency in inducing proliferation of CD8+ and CD4+ T cells in comparison to XENP24635, recombinant IL-2, and recombinant IL-15.

In a proliferation assay, PBMCs were incubated with indicated concentrations of indicated test articles with indicated concentration of plate-bound anti-CD3 mAb (OKT3). Lymphocyte populations were stained as follows: anti-FoxP3-PE (PCH101), anti-CD8-PerCP-Cγ5.5 (SKI), anti-CD3-PE-Cγ7 (OKT3), anti-Ki67-APC, anti-CD45RO-Alexa700 (UCHL1), anti-CD25− BV421 (M-A251), anti-CD16-BV605 (3G6), anti-CD56-BV605 (HCD56), anti-CD45RA-BV785 (HI100), anti-CD4-BUV396 (SK3), and Zombie NIR (APC-Cγ7). Proliferation of various lymphocyte populations were determined based on percentage expressing K167, a proliferation marker, data for which are depicted in FIGS. 55-65. Consistent with the data depicted above from the suppression assay, the data from the proliferation assay as depicted in FIGS. 55-61 show that XENP27563 and XENP27564 (IL-2-Fc fusions engineered for CD25− selectivity) have impaired function in inducing proliferation of CD8+ T cells, CD8+CD45RA− T cells, CD8+CD45RA+ T cells, CD4+ T cells, CD4+CD45RA− T cells, CD4+CD45RA+ T cells, NK cells; and the data as depicted in FIGS. 63-65 show that XENP27563 and XENP27564 selectively expand Tregs over the other lymphocyte populations.

### H. Example 8: IL-2-Fc Fusions Promote Selective and Sustained Treg Expansion in Cynomolgus Monkeys

To investigate the clinical potential of XENP27563 and XENP27564, we investigated their activity in cynomolgus monkeys. Prior to dosing animals, we confirmed the activity of the IL-2-Fc fusions on cynomolgus lymphocytes. We performed two assays, described as follows.

In a first assay, human PBMCs were stimulated with various concentrations of XENP27563 or XENP27564 for 15 minutes at 37° C. PBMCs were then stained with anti_CD3-BUV395 (UCHT1), anti-CD4-BV605 (RPA-T4), anti-CD8-BV711 (RPA-T8), anti-CD25-BV421 (M-A251), anti-CD45RA-BV510 (HI100), and anti-CD56-PE. Cells were then permeabilized using PerFix EXPOSE (Beckman Coulter, Indianapolis, Ind.). Following permeabilization, cells were stained with anti-CD16-AF700 (DJ130C), anti-FoxP3-AF488 (259D), and pSTAT5 (pY694) and analyzed by flow cytometry for STAT5 phosphorylation on various lymphocyte populations, data for which are depicted in FIG. 78.

In a second assay, cynomolgus PBMCs were mulated with various concentrations of XENP27563 or XENP27564 for 15 minutes at 37° C. PBMCs were then stained with anti_CD3-BV421 (SP34), anti-CD4-BV785 (OKT4), anti-CD8-BUV395 (RPA-T8), anti-CD25-BV510 (M-A251), anti-CD45RA-APC/H7 (HI100), and anti-CD56-PE. Cells were then permeabilized using PerFix EXPOSE (Beckman Coulter, Indianapolis, Ind.). Following permeabilization, cells were stained with anti-CD16-AF700 (DJ130C), anti-FoxP3-AF488 (259D), and pSTAT5 (pY694) and analyzed by flow cytometry for STAT5 phosphorylation on various lymphocyte populations, data for which are depicted in FIG. 79.

The data show that XENP27563 and XENP27564 are equally selective and potent for both human and cynomolgus Tregs.

### 1. 8A: Comparing the PD and PK of XENP27563 and XENP27564

In a first study in cynomolgus monkeys, animals (n=2) were intravenously dosed with either 3× dose XENP27563 or 3× dose XENP27564 on Days 0 and 15. Blood was collected over time to investigate the expansion of various lymphocyte populations and to investigate the serum concentration of the test articles. Serum albumin concentration and blood pressure of animals were also taken to investigate the tolerability of the test articles.

FIGS. 66A-66D depicts the expansion of various lymphocyte populations over time. The data show that both test articles were able to expand Tregs, while maintaining close to baseline levels of CD8+CD45RA− T cells, CD4+CD45RA− T cells, and CD16+ NK cells. Further, the data show that the two test articles promote similar pharmacology in monkeys. FIGS. 67A-67B depicts the serum concentration of test articles. The data show that the two test articles demonstrated similar pharmacokinetic profiles, with a half-life of 1.5 days for XENP27564. Taken together, this supports that IL-2-Fc fusions engineered for CD25-selectivity and reduced potency provide selective and sustained Treg expansion.

Vascular leak syndrome is a hallmark toxic side-effect associated with treatment with cytokines such as IL-2. One indication of vascular leak is hypoalbuminemia, a drop in serum albumin concentration. Accordingly, we investigated the change in serum albumin concentration in the animals, data for which are depicted in FIG. 68. Notably in one animal dosed with XENP27563, long-lasting albumin drop was detected after both the first and the second dosing. In one animal dosed with XENP27564, an albumin drop was detected after the second dosing, but concentration quickly recovered to baseline levels. This data suggests that XENP27564, having lower potency than XENP27563, may promote superior tolerability and therapeutic index.

Another indicator of vascular leak is sharp drops in blood pressure. Accordingly, we recorded the blood pressure for animals on Days 0, 1, 3, 5, 9, 16, 18, 20, and 24, data for which are depicted in FIGS. 69A-69C. Notably, a first monkey dosed with XENP27563 suffered blood pressure drop on Day 1 (1 day after 1st dose), and a second monkey dosed with XENP27563 suffered blood pressure drop on Day 16 (1 day after 2nd dose), while stable blood pressure was observed on all days for a monkey dosed with XENP27564 (telemetry data was corrupt for 2nd monkey dosed with XENP27564). This further supports the notion that lower potency IL-2-Fc fusions may promote superior tolerability and therapeutic index.

Finally, we also investigated eosinophil and basophil counts as additional indicators of tolerability, data for which are depicted in FIGS. 76A-76B. Collectively, the data show that repeat dosing with XENP27564.

### 2. 8B: XENP27564 Dose Escalation Study

In a first study in cynomolgus monkeys, animals (n=3) were intravenously dosed with either 1×, 3×, or 10× dose XENP27564. Blood was collected over time to investigate the expansion of various lymphocyte populations, as well as concentration of serum albumin and C-Reactive Protein (CRP).

Expansion of various lymphocyte populations are depicted in FIGS. 70A-71. Consistent with the data from the first cynomolgus study, XENP27564 provides selective and sustained Treg expansion. Further, the data show that 1× and 3× dose promote similar pharmacology (as indicated by Treg expansion) in monkeys. Notably, higher dose (10× dose) of XENP27564 did not enhance pharmacodynamics. Consistent with the data depicted above, FIG. 75 show sustained pharmacokinetics up to several days in cynomolgus monkeys at all tested doses of XENP27564.

As in our first study, we investigated albumin drop as an indicator of vascular leak and tolerability, data for which are depicted in FIG. 72. Further, we investigated serum concentration of CRP, an acute-phase protein associated with inflammation, as another indicator of tolerability, data for which are depicted in FIG. 73. We also investigated sodium concentration, chloride concentration, eosinophil counts, and basophil counts as additional indicators of tolerability (data for which are depicted in FIGS. 74A-74D). Notably, the data show that higher doses of XENP27564 increased toxicity as indicated by both albumin drop and increase in serum CRP concentrations, (as well as sodium concentration, chloride concentration, eosinophil counts, and basophil counts), however lower doses which still resulted in significant increases in Tregs were more tolerable.

### I. Example 9: IL-2-Fc Fusions are Equally Selective for Tregs in Mice

Splenocytes from B6 mice were incubated with IL-2-Fc fusions and recombinant human IL-2 for 15 minutes. Following incubation, cells were stained with anti-CD4-PE (GK1.5), anti-CD25-BV605 (PC61), and anti-CD44-BV510 (IM7). Cells were then permeabilized using PerFix EXPOSE (Beckman Coulter, Indianapolis, Ind.). Following permeabilization, cells were stained with anti_CD3-AF700 (2C11), anti-CD8-AF488 (53-6.7), anti-FoxP3-eF450 (FJK-16S), and anti-pSTAT5 (pY694) and analyzed by flow cytometry for STAT5 phosphorylation on various lymphocyte populations, data for which are depicted in FIGS. 77A-77C. The data indicates that the engineered IL-2-Fc fusions are equally selective and potent for Tregs in mice making it suitable for using preclinical mouse models to investigate autoimmune diseases.

1.-18. (canceled)

19. A variant human IL-2, wherein said variant IL-2 comprises amino acid substitution(s) as compared to SEQ ID NO:2 selected from the group consisting of T3A/D20N; T3A/R38E; T3A/C125A; D20N/R38E; D20N/C125A; R38E/C125A; T3A/D20N/R38E; T3A/D20N/C125A; T3A/R38E/C125A; D20N/R38E/C125A; T3A/D20N/R28E/C125A; T3A/E61Q; T3A/E62Q; T3A/C125A; E61Q/E62Q; E61Q/C125A; E62Q/C125A; T3A/E61Q/E62Q; T3A/E61Q/C125A; T3A/E62Q/C125A; E61Q/E62Q/C125A; T3A/E61Q/E62Q/C125A; T3A/F42Y; T3A/E62Q; T3A/C125A; F42Y/E62Q; F42Y/C125A; E62Q/C125A; T3A/F42Y/E62Q; T3A/F42Y/C125A; T3A/E62Q/C125A; F42Y/E62Q/C125A; T3A/F42Y/E62Q/C125A; T3A/D20N; T3A/F42Y; T3A/E62Q; T3A/C125A; D20N/F42Y; D20N/E62Q; D20N/C125A; F42Y/E62Q; F42Y/C125A; E62Q/C125A; T3A/D20N/F42Y; T3A/D20N/E62Q; T3A/D20N/C125A; T3A/F42Y/E62Q; T3A/F42Y/C125A; T3A/E62Q/C125A; D20N/F42Y/E62Q; D20N/F42Y/C125A; D20N/E62Q/C125A; F42Y/E62Q/C125A; T3A/D20N/F42Y/E62Q; T3A/D20N/F42Y/C125A; T3A/D20N/E62Q/C125A; T3A/F42Y/E62Q/C125A; D20N/F42Y/E62Q/C125A; T3A/D20N/F42Y/E62Q/C125A; T3A/F42A; T3A/N88D; T3A/C125A; F42A/N88D; F42A/C125A; N88D/C125A; T3A/F42A/N88D; T3A/F42A/C125A; T3A/N88D/C125A; F42A/N88D/C125A; T3A/F42A/N88D/C125A; T3A/F42A; T3A/C125A; F42A/C125A; T3A/F42A/C125A; T3A/F42Y; T3A/E62Q; T3A/N88R; T3A/C125A; F42Y/E62Q; F42Y/N88R; F42Y/C125A; E62Q/N88R; E62Q/C125A; N88R/C125A; T3A/F42Y/E62Q; T3A/F42Y/N88R; T3A/F42Y/C125A; T3A/E62Q/N88R; T3A/E62Q/C125A; T3A/N88R/C125A; F42Y/E62Q/N88R; F42Y/E62Q/C125A; F42Y/N88R/C125A; E62Q/N88R/C125A; T3A/F42Y/E62Q/N88R; T3A/F42Y/E62Q/C125A; T3A/F42Y/N88R/C125A; T3A/E62Q/N88R/C125A; F42Y/E62Q/N88R/C125A; T3A/F42Y/E62Q/N88R/C125A; T3A/D20N; T3A/F42Y; T3A/E62Q; T3A/C125A; D20N/F42Y; D20N/E62Q; D20N/C125A; F42Y/E62Q; F42Y/C125A; E62Q/C125A; T3A/D20N/F42Y; T3A/D20N/E62Q; T3A/D20N/C125A; T3A/F42Y/E62Q; T3A/F42Y/C125A; T3A/E62Q/C125A; D20N/F42Y/E62Q; D20N/F42Y/C125A; D20N/E62Q/C125A; F42Y/E62Q/C125A; T3A/D20N/F42Y/E62Q; T3A/D20N/F42Y/C125A; T3A/D20N/E62Q/C125A; T3A/F42Y/E62Q/C125A; D20N/F42Y/E62Q/C125A; T3A/D20N/F42Y/E62Q/C125A; T3A/D20N; T3A/E61Q; T3A/E62Q; T3A/C125A; D20N/E61Q; D20N/E62Q; D20N/C125A; E61Q/E62Q; E61Q/C125A; E62Q/C125A; T3A/D20N/E61Q; T3A/D20N/E62Q; T3A/D20N/C125A; T3A/E61Q/E62Q; T3A/E61Q/C125A; T3A/E62Q/C125A; D20N/E61Q/E62Q; D20N/E61Q/C125A; D20N/E62Q/C125A; E61Q/E62Q/C125A; T3A/D20N/E61Q/E62Q; T3A/D20N/E61Q/C125A; T3A/D20N/E62Q/C125A; T3A/E61Q/E62Q/C125A; D20N/E61Q/E62Q/C125A; and T3A/D20N/E61Q/E62Q/C125A.

20. A heterodimeric protein comprising a first monomer comprising a variant IL-2 covalently attached to a first variant Fc domain and a second monomer comprising a second variant Fc domain, wherein said variant IL-2 comprises amino acid substitution(s) as compared to SEQ ID NO:2 selected from the group consisting of T3A/D20N; T3A/R38E; T3A/C125A; D20N/R38E; D20N/C125A; R38E/C125A; T3A/D20N/R38E; T3A/D20N/C125A; T3A/R38E/C125A; D20N/R38E/C125A; T3A/D20N/R28E/C125A; T3A/E61Q; T3A/E62Q; T3A/C125A; E61Q/E62Q; E61Q/C125A; E62Q/C125A; T3A/E61Q/E62Q; T3A/E61Q/C125A; T3A/E62Q/C125A; E61Q/E62Q/C125A; T3A/E61Q/E62Q/C125A; T3A/F42Y; T3A/E62Q; T3A/C125A; F42Y/E62Q; F42Y/C125A; E62Q/C125A; T3A/F42Y/E62Q; T3A/F42Y/C125A; T3A/E62Q/C125A; F42Y/E62Q/C125A; T3A/F42Y/E62Q/C125A; T3A/D20N; T3A/F42Y; T3A/E62Q; T3A/C125A; D20N/F42Y; D20N/E62Q; D20N/C125A; F42Y/E62Q; F42Y/C125A; E62Q/C125A; T3A/D20N/F42Y; T3A/D20N/E62Q; T3A/D20N/C125A; T3A/F42Y/E62Q; T3A/F42Y/C125A; T3A/E62Q/C125A; D20N/F42Y/E62Q; D20N/F42Y/C125A; D20N/E62Q/C125A; F42Y/E62Q/C125A; T3A/D20N/F42Y/E62Q; T3A/D20N/F42Y/C125A; T3A/D20N/E62Q/C125A; T3A/F42Y/E62Q/C125A; D20N/F42Y/E62Q/C125A; T3A/D20N/F42Y/E62Q/C125A; T3A/F42A; T3A/N88D; T3A/C125A; F42A/N88D; F42A/C125A; N88D/C125A; T3A/F42A/N88D; T3A/F42A/C125A; T3A/N88D/C125A; F42A/N88D/C125A; T3A/F42A/N88D/C125A; T3A/F42A; T3A/C125A; F42A/C125A; T3A/F42A/C125A; T3A/F42Y; T3A/E62Q; T3A/N88R; T3A/C125A; F42Y/E62Q; F42Y/N88R; F42Y/C125A; E62Q/N88R; E62Q/C125A; N88R/C125A; T3A/F42Y/E62Q; T3A/F42Y/N88R; T3A/F42Y/C125A; T3A/E62Q/N88R; T3A/E62Q/C125A; T3A/N88R/C125A; F42Y/E62Q/N88R; F42Y/E62Q/C125A; F42Y/N88R/C125A; E62Q/N88R/C125A; T3A/F42Y/E62Q/N88R; T3A/F42Y/E62Q/C125A; T3A/F42Y/N88R/C125A; T3A/E62Q/N88R/C125A; F42Y/E62Q/N88R/C125A; T3A/F42Y/E62Q/N88R/C125A; T3A/D20N; T3A/F42Y; T3A/E62Q; T3A/C125A; D20N/F42Y; D20N/E62Q; D20N/C125A; F42Y/E62Q; F42Y/C125A; E62Q/C125A; T3A/D20N/F42Y; T3A/D20N/E62Q; T3A/D20N/C125A; T3A/F42Y/E62Q; T3A/F42Y/C125A; T3A/E62Q/C125A; D20N/F42Y/E62Q; D20N/F42Y/C125A; D20N/E62Q/C125A; F42Y/E62Q/C125A; T3A/D20N/F42Y/E62Q; T3A/D20N/F42Y/C125A; T3A/D20N/E62Q/C125A; T3A/F42Y/E62Q/C125A; D20N/F42Y/E62Q/C125A; T3A/D20N/F42Y/E62Q/C125A; T3A/D20N; T3A/E61Q; T3A/E62Q; T3A/C125A; D20N/E61Q; D20N/E62Q; D20N/C125A; E61Q/E62Q; E61Q/C125A; E62Q/C125A; T3A/D20N/E61Q; T3A/D20N/E62Q; T3A/D20N/C125A; T3A/E61Q/E62Q; T3A/E61Q/C125A; T3A/E62Q/C125A; D20N/E61Q/E62Q; D20N/E61Q/C125A; D20N/E62Q/C125A; E61Q/E62Q/C125A; T3A/D20N/E61Q/E62Q; T3A/D20N/E61Q/C125A; T3A/D20N/E62Q/C125A; T3A/E61Q/E62Q/C125A; D20N/E61Q/E62Q/C125A; and T3A/D20N/E61Q/E62Q/C125A.

21. A heterodimeric protein according to claim 19 or 20, wherein said variant Fc domains are variant human IgG1 Fc domains comprising the amino acid substitutions M428L/N434S.

22. A heterodimeric protein according to claim 19 or 20, wherein said variant Fc domains are variant human IgG1 Fc domains comprising the amino acid substitutions E233P/L234V/L235A/G236del/S267K.

23. A heterodimeric protein according to claim 19 or 20, wherein said first and second variant Fc domains comprise a set of heterodimerization variants selected from the group consisting of those depicted in FIG. 2.

24. A heterodimeric protein according to claim 23, wherein said set of heterodimerization variants selected from the group consisting of L368D/K370S:S364K/E357Q; L368D/K370S:S364K; L368E/K370S:S364K; T411E/K360E/Q362E:D401K; and T366S/L368A/Y407V:T366W.

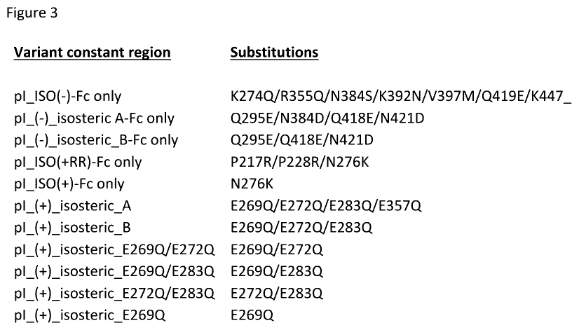

25. A heterodimeric protein according to claim 19 or 20, wherein one of said variant Fc domains comprise a set of pI variants selected from the group consisting of those depicted in FIG. 3.

26. A heterodimeric protein according to claim 25, wherein said pI variant set is A295E/N384D/Q418/N421D.

27. A heterodimeric protein according to claim 19 or 20, wherein said variant Fc domain contains a number of amino acid modifications as compared to the parental human IgG1, IgG2 or IgG4.

28. A nucleic acid encoding the variant human IL-2 according to claim 19.

29. An expression vector comprising the nucleic acid of claim 28.

30. A host cell comprising the expression vector of claim 29.

31. A method of making a variant human IL-2 comprising culturing the host cell of claim 30 under conditions wherein the variant human IL-2 is produced and recovering the variant human IL-2.

32. A nucleic acid composition comprising:
a. a first nucleic acid encoding said first monomer of claim 20; and
b. a second nucleic acid encoding said second monomer of claim 20, respectively.

33. An expression vector composition comprising:
a. a first expression vector comprising said first nucleic acid of claim 32; and
b. a second expression vector comprising said second nucleic acid of claim 32.

34. A host cell comprising the expression vector composition according to claim 33.

35. A method of making a polypeptide composition comprising culturing the host cell of claim 33 under conditions wherein the composition is produced and recovering the composition.
